(19) United States
(12) Patent Application Publication
(10) Pub. No.: US 2006/0014231 A1
(43) Pub. Date: Jan. 19, 2006

(54) Methods and compositions to promote bone homeostasis

(76) Inventors: Luc Van Rompaey, Keerbergen (BE); Peter Herwig Maria Tomme, Gent (BE)

Correspondence Address: SYNNESTVEDT & LECHNER, LLP, 2600 ARAMARK TOWER, 1101 MARKET STREET, PHILADELPHIA, PA, 191072950, US

(21) Appl. No.: 11/166,412

(22) Filed: Jun. 24, 2005

Related U.S. Application Data
(60) Provisional application No. 60/582,704, filed on Jun. 24, 2004.
(60) Provisional application No. 60/630,449, filed on Nov. 23, 2004.
(60) Provisional application No. 60/673,206, filed on Apr. 20, 2005.

(51) Int. Cl. C12Q 1/42

(52) U.S. Cl. 435/21

(57) ABSTRACT

Methods are disclosed for identifying osteogenic promoting compounds by contacting test compounds with a target gene polypeptide or fragment thereof, which target gene is identified as involved in the osteogenesis process, and measuring a compound-polypeptide osteogenesis property. Also disclosed are methods of promoting osteogenesis by contacting progenitor cells with an effective osteogenic stimulating amount of an agonist of a target gene or an expressible nucleic acid of SEQ ID NO. 1-18, and may be used for the treatment or prevention of an imbalance in bone homeostasis. A further aspect is a method to produce bone tissue in vitro, by contacting a target gene agonist or an expressible nucleic acid of SEQ ID NO. 1-18 with a vertebrate cell population including osteoblast progenitor cells on a substrate.

### CROSS-REFERENCE TO RELATED APPLICATIONS

This application claims priority to U.S. Provisional Application No. 60/582,704, filed Jun. 24, 2004, U.S. Provisional Application No. 60/630,449, filed Nov. 23, 2004, and U.S. Provisional Application No. 60/673,206, filed Apr. 20, 2005, the disclosures of which are incorporated herein by reference.

### FIELD OF INVENTION

This invention relates to the field of bone metabolism, and in particular, to methods, therapies, and compositions useful, for the prevention and treatment of diseases associated with an imbalance, or disturbance, in bone homeostasis in humans and other animals.

Bone is a dynamic tissue that is continuously being destroyed (resorbed) and rebuilt, by an intricate interplay between two distinct cell lineages: bone-forming cells, known as osteoblasts and bone-resorbing cells, known as osteoclasts. The cascade of transcription factors and growth factors involved in the differentiation or progression from progenitor cell to functional osteoclast is well established. In contrast, little is known about the factors involved in the progression of osteoblasts from progenitor cells. The mesenchymal progenitor or stem cells (MPCs) represent the starting points for the differentiation of both osteoclasts and osteoblasts. During embryonic development in vivo, bone formation occurs through two distinct pathways: intramembranous and/or endochondral ossification (see FIG. 1; taken from Nakashima and de Crombrugghe, (2003)). During intramembranous ossification, flat bones such as those of the skull or clavicles, are formed directly from condensations of mesenchymal cells. During endochondral ossification, long bones, such as limb bones, are formed from a cartilage intermediate formed during mesenchymal condensation, which intermediate is invaded during further development by endothelial cells, osteoclasts and mesenchymal cells that further differentiate into osteoblasts and osteocytes. During this latter differentiation into osteoblasts, bone alkaline phosphatase activity (BAP) is up-regulated.

A number of diseases are the direct result of a disturbance in the fine-tuned balance between bone resorption and bone formation. These diseases for the most part are skeletal diseases and inflict a large number of patients. Exemplary diseases include hypocalcaemia of malignancy, Paget's disease, inflammatory bone diseases such as rheumatoid arthritis and periodontal disease, focal osteogenesis occurring during skeletal metastases, Crouzon's syndrome, rickets, opsismodysplasia, pycnodysostosis/Toulouse-Lautrec disease, osteogenesis imperfecta, and osteoporosis. The single most prevalent bone disease is osteoporosis, which affects 1 in 5 women over 50 and 1 in 20 men over 50.

### Reported Developments

A number of treatments have been developed and made available to patients suffering from osteoporosis and related skeletal diseases. These therapeutic approaches primarily are directed to increasing net bone formation and include: hormone replacement therapy (HRT); selective estrogen receptor modulators (SERMs); bisphosphonates; and calcitonin. While these treatments slow down bone resorption, they don't abolish fracturing because the lost bone is not sufficiently replenished. Fracturing will be prevented only if bone formation is sufficiently increased. Therefore, there is great interest in identifying osteogenic pathways that enhance bone anabolism as a basis for therapeutic intervention.

Parathyroid hormone (PTH) 1-34 is the only bone anabolic therapy on the osteoporosis therapeutic market. While PTH displays bone anabolic effects when administered intermittently, it needs to be injected daily, and may have tumorgenic side effects, based on the observation that tumors form in animals treated with at PTH in high doses.

Bone morphogenetic proteins (BMPs) are another class of bone anabolic therapeutics, but have only been approved for niche markets. Receptors for the bone morphogenetic proteins have been identified in many tissues other than bone, and BMPs themselves are expressed in a large variety of tissues in specific temporal and spatial patterns. This suggests that BMPs may have effects on many tissues other than bone, potentially limiting their usefulness as therapeutic agents when administered systemically.

There is a clear need to identify additional targets that stimulate osteogenic differentiation and that can be used for the development of novel bone anabolic therapies.

The present invention is based on the discovery of that certain known polypeptides, including the GPCR and NHR peptides, are factors in the up-regulation and/or induction of osteogenic differentiation in bone marrow cells, and that the known agonists for these polypeptides are effective in promoting bone homeostasis.

### SUMMARY OF THE INVENTION

The present invention relates to a method for identifying a compound that promotes osteogenesis in a population of vertebrate cells including osteoblast progenitor cells, comprising contacting a compound with a polypeptide comprising an amino acid sequence selected from the group consisting of SEQ ID NO: 101-118 and 201-363; and measuring a compound-polypeptide property related to osteogenesis.

The invention also relates to methods, and compositions useful in these methods, of promoting osteogenic differentiation in a subject suffering or susceptible to an imbalance in bone homeostasis, comprising administering to said subject a therapeutically effective amount of an agonist of a polypeptide comprising an amino acid sequence selected from the group consisting of SEQ ID NO: 101-118 and 201-363, or an expressible nucleic acid sequence encoding a polypeptide comprising an amino acid sequence selected from the group consisting of SEQ ID NO: 101-118.

Another aspect of the present invention relates to a method for in vitro production of bone tissue comprising applying undifferentiated vertebrate cells onto a substrate to form a cellular layered article, and contacting a polynucleotide comprising a expressible nucleic acid sequence selected from the group consisting of SEQ ID NO: 1-18 with said article for a time sufficient to differentiate said undifferentiated cells into osteoblasts, thereby producing a matrix containing osteoblast cells.

### BRIEF DESCRIPTION OF THE DRAWINGS

FIG. 1. Intramembranous and endochondral ossification.

FIG. 2. Principle of the osteoblast differentiation assay.

FIG. 3. Performance of the knock-in control plate in the AP assay.

FIG. 4. Dot plot representation of raw data for one FLeXeSelect screening plate.

FIG. 5. Dose-dependent up-regulation of AP activity by selected compounds.

FIG. 6. Analyzing the up-regulation of BAP-mRNA versus PLAP- or IAP-mRNA.

FIG. 7. Mineralization of primary human MPCs.

FIG. 8. Mineralization of primary human MPCs.

FIG. 9. Dose-dependent up-regulation of AP activity by the LXR agonist GW3965 in the presence of Ad-NR1H3.

FIG. 10. Dose-dependent up-regulation of AP activity by the LXR agonist T0901317 in the presence of Ad-NR1H2.

FIG. 11. Dose-dependent up-regulation of AP activity by the LXR agonist GW3965 in the presence of Ad-NR1H2.

FIG. 12. Structure of the acetyl podocarpic dimer (APD) used in this application.

FIG. 13. Dose-dependent up-regulation of AP activity by the LXR agonist APD in the presence of Ad-NR1H2 or Ad-NR1H3.

FIG. 14A-14D. Ct values and relative expression levels of four genes of the present invention compared to beta-actin for cell types relevant to bone formation.

FIG. 15. NR5A2 and NR1H3+T0901317 up-regulate mRNA levels of osteogenic markers.

FIG. 16. Up-regulation of NR5A2 and NR1H3 mRNA levels by osteogenic triggers.

FIG. 17. Weight increases in calvarial skull explants induced by the positive controls Ad-BMP2 and Ad-BMP7.

FIG. 18. Weight increases in calvarial skull explants induced by T0901317.

FIG. 19. DN-RUNX2 interferes with induction of AP activity by NR5A2, NR1H3+T0901317 and ESRRG.

FIG. 20. NR5A2, NR1H3+T0901317, and ESRRG induce AP activity independent of the MPC isolate.

### DETAILED DESCRIPTION

The following terms are intended to have the meanings presented therewith below and are useful in understanding the description of and intended scope of the present invention.

The term “agonist” refers to a ligand that stimulates the receptor the ligand binds to in the broadest sense.

The term “carrier” means a non-toxic material used in the formulation of pharmaceutical compositions to provide a medium, bulk and/or useable form to a pharmaceutical composition. A carrier may comprise one or more of such materials such as an excipient, stabilizer, or an aqueous pH buffered solution. Examples of physiologically acceptable carriers include aqueous or solid buffer ingredients including phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as TWEEN™, polyethylene glycol (PEG), and PLURONICS™.

The term “compound” is used herein in the context of a “test compound” or a “drug candidate compound” described in connection with the assays of the present invention. As such, these compounds comprise organic or inorganic compounds, derived synthetically or from natural sources. The compounds include inorganic or organic compounds such as polynucleotides, lipids or hormone analogs that are characterized by relatively low molecular weights. Other biopolymeric organic test compounds include peptides comprising from about 2 to about 40 amino acids and larger polypeptides comprising from about 40 to about 500 amino acids, such as antibodies or antibody conjugates.

The term “contact” or “contacting” means bringing at least two moieties together, whether in an in vitro system or an in vivo system.

The term “condition” or “disease” means the overt presentation of symptoms (i.e., illness) or the manifestation of abnormal clinical indicators (e.g., biochemical indicators), resulting from defects in one amyloid beta protein precursor processing. Alternatively, the term “disease” refers to a genetic or environmental risk of or propensity for developing such symptoms or abnormal clinical indicators.

The term “effective amount” means that amount of a drug or pharmaceutical agent that will elicit the biological or medical response of a subject that is being sought by a medical doctor or other clinician. In particular, with regard to treating an imbalance in bone homeostasis, the term “effective osteogenic stimulating amount” is intended to mean that effective amount of an LXR agonist or prodrug of LXR agonist that will bring about a biologically meaningful increase in the ratio of osteoblasts to osteoclasts in the subject's bone tissue. A biologically meaningful increase is that increase that can be detected indirectly by means of bone density, bone strength, or other diagnostic indicia known to those skilled in the art.

The term “expression” relates to both endogenous expression and over-expression by for instance transfection or stable transduction.

The term “GPCR” means a G-protein coupled receptor. Preferred GPCRs comprise those receptors identified by applicants as promoting osteogenic differentiation. Most preferred GPCRs are those identified in Table 1, including the naturally occurring transcript variants thereof.

The term “ligand” means an endogenous, naturally occurring molecule specific for an endogenous, naturally occurring receptor.

The term “LXR” includes all subtypes of this receptor as known in the prior art and corresponding genes that encode such subtypes. Specifically LXR includes LXR-alpha and LXR-beta, and an agonist of LXR should be understood to include an agonist of LXR-alpha or LXR-beta. LXR-alpha is referred to under a variety of names and for purposes of this application LXR-alpha should be understood to mean any gene referred to as LXR-alpha, LXRₐ, LXRα, RLD-1, NR1H3 or a gene with homology to accession number U22662 or a protein with homology to a protein encoded by such a polynucleotide. Similarly, LXR-beta should be understood to include any gene referred to as LXR_{b}, LXR-beta, LXRbeta, NER, NER1, UR, OR-1, R1P15, NR1H2 or a gene with homology to accession number U07132 or a protein with homology to a protein encoded by such a polynucleotide. “Homology” means sequence similarity to the extent that polynucleotides of the “homologous” sequence are able to hybridize to the LXR sequence under stringent hybridization conditions as understood by a person of skill in the art.

The term “NHR” means a nuclear hormone receptor.

The term “osteogenesis” means a process that consists of several successive events, including initially the up-regulation of bone alkaline phosphatase in a cell, and calcium deposition (mineralization) which occurs in later stages of process.

The term “osteogenic differentiation” refers to any process wherein unspecialized cells in a lineage of bone-related cells become more specialized by exhibiting anabolic processes resulting in the deposition of calcium and the formation of bone tissue.

The term “pharmaceutically acceptable carrier” includes, for example, pharmaceutically acceptable carriers such as the following: solid carriers such as lactose, magnesium stearate, terra alba, sucrose, talc, stearic acid, gelatin, agar, pectin, acacia or the like; and liquids such as vegetable oils, arachis oil and sterile water, or the like. However, this listing of pharmaceutically acceptable carriers is not to be construed as limiting.

The term “pharmaceutically acceptable salts” refers to the non-toxic, inorganic and organic acid addition salts, and base addition salts, of compounds of the present invention. These salts can be prepared in situ during the final isolation and purification of compounds useful in the present invention.

The term “polynucleotide” refers to nucleic acids, such as double stranded, or single stranded DNA and (messenger) RNA, and all types of oligonucleotides. It also includes nucleic acids with modified backbones such as peptide nucleic acid (PNA), polysiloxane, and 2′-O-(2-methoxy)ethylphosphorothioate. “Derivatives of a polynucleotide” means DNA-molecules, RNA-molecules, and oligonucleotides that comprise a stretch or nucleic acid residues of the polynucleotide, e.g. polynucleotides that may have nucleic acid mutations as compared to the nucleic acid sequence of a naturally occurring form of the polynucleotide. A derivative may further comprise nucleic acids with modified backbones such as PNA, polysiloxane, and 2′-O-(2-methoxy)ethyl-phosphorothioate, non-naturally occurring nucleic acid residues, or one or more nucleic acid substituents, such as methyl-, thio-, sulphate, benzoyl-, phenyl-, amino-, propyl-, chloro-, and methanocarbanucleosides, or a reporter molecule to facilitate its detection. “Fragment of a polynucleotide” means oligonucleotides that comprise a stretch of contiguous nucleic acid residues that exhibit substantially a similar, but not necessarily identical, activity as the complete sequence.

The term “polypeptide” relates to proteins, proteinaceous molecules, fractions of proteins, peptides, oligopeptides, and enzymes (such as kinases, proteases, GCPRs). “Derivatives of a polypeptide” relate to those peptides, oligopeptides, polypeptides, proteins and enzymes that comprise a stretch of contiguous amino acid residues of the polypeptide and that retain the biological activity of the protein, e.g. polypeptides that have amino acid mutations compared to the amino acid sequence of a naturally-occurring form of the polypeptide. A derivative may further comprise additional naturally occurring, altered, glycosylated, acylated or non-naturally occurring amino acid residues compared to the amino acid sequence of a naturally occurring form of the polypeptide. It may also contain one or more non-amino acid substituents compared to the amino acid sequence of a naturally occurring form of the polypeptide, for example a reporter molecule or other ligand, covalently or non-covalently bound to the amino acid sequence. “Fragment of a polypeptide” relates to peptides, oligopeptides, polypeptides, proteins and enzymes that comprise a stretch of contiguous amino acid residues, and exhibit substantially a similar, but not necessarily identical, functional activity as the complete sequence.

The term “solvate” means a physical association of a compound useful in this invention with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

The term “subject” includes humans and other mammals.

The term “treating” refers to alleviating the disorder or condition to which the term “treating” applies, including one or more symptoms of such disorder or condition. The related term “treatment,” as used herein, refers to the act of treating a disorder, symptom, or condition, as the term “treating” is defined above.

The term “vectors” also relates to plasmids as well as to viral vectors, such as recombinant viruses, or the nucleic acid encoding the recombinant virus.

The term “vertebrate cells” means cells derived from animals having vertera structure, including fish, avian, reptilian, amphibian, marsupial, and mammalian species. Preferred cells are derived from mammalian species, and most preferred cells are human cells. Mammalian cells include feline, canine, bovine, equine, caprine, ovine, porcine murine, such as mice and rats, and rabbits.

The Methods of the Present Invention

The present invention relates to methods for increasing and/or inducing osteogenesis, or more particularly, osteogenic differentiation of osteoblast progenitor cells, said method comprising contacting (1) a population of vertebrate cells expressing a polypeptide encoded by a target gene identified in Table 1 below, or a functional fragment or derivative thereof, some of which are identified in Table 1A below; with (2) an agonist for such target gene; and (3) thereby increasing the level of osteogenic differentiation in said population of cells.

TABLE 1
List of identified target genes.

| | | | GenBank | SEQ | GenPept | SEQ |
|---|---|---|---|---|---|---|
| | | | accession | ID | accession | ID |
| Gene symbol | Gene description | Class | (DNA) | DNA | (Protein) | Protein |
| ADORA2A | adenosine A2a receptor | GPCR | NM_000675 | 1 | NP_000666 | 101 |
| NR1H3 | nuclear receptor subfamily 1, group H, | NHR | NM_005693 | 2 | NP_005684 | 102 |
| | member 3 | | | | | |
| HSU93553/ | alpha1-fetoprotein transcription factor | NHR | U93553 | 3 | AAD03155 | 103 |
| NR5A2 | (hFTF) | | NM_003822 | 4 | NP_003813 | 104 |
| | | | NM_205860 | 5 | NP_995582 | 105 |
| GPR52 | G protein-coupled receptor 52 | GPCR | NM_005684 | 6 | NP_005675 | 106 |
| RE2/GPR161 | G protein-coupled receptor 161 | GPCR | NM_007369 | 7 | NP_031395 | 107 |
| | | | NM_153832 | 8 | NP_722561 | 108 |
| | | | 3273814CA2 | 9 | 3273814CA2 | 109 |
| GPR65 | G protein-coupled receptor 65 | GPCR | NM_003608 | 10 | NP_003599 | 110 |
| ESRRG | estrogen-related receptor gamma | NHR | NM_001438 | 11 | NP_001429 | 111 |
| | | | NM_206594 | 12 | NP_996317 | 112 |
| | | | NM_206595 | 13 | NP_996318 | 113 |
| GPR12 | G protein-coupled receptor 12 | GPCR | NM_005288 | 14 | NP_005279 | 114 |
| MC5R | melanocortin 5 receptor | GPCR | NM_005913 | 15 | NP_005904 | 115 |
| AVPR2 | arginine vasopressin receptor 2 | GPCR | NM_000054 | 16 | NP_000045 | 116 |
| | (nephrogenic diabetes insipidus) | | | | | |
| DRD1 | dopamine receptor D1 | GPCR | NM_000794 | 17 | NP_000785 | 117 |
| NR1H2 | nuclear receptor subfamily 1, group H, | NHR | NM_007121 | 18 | NP_009052 | 118 |
| | member 2 | | | | | |

Methods Used to Identify Relationship between Target Genes and Osteogenic Differentiation

The above-identified osteogenic differentiation-related target genes were identified using a so-called ‘knock-in’ library in the following manner. Using recombinant adenoviruses, the present inventors tranduced cDNA molecules coding for a specific natural gene and gene product into cells. Each cDNA introduced into each separate subpopulation of cells induced the expression and activity of the corresponding gene and gene product in a cell. By identifying a cDNA that induces or increases osteogenic differentiation, a direct link is made to the corresponding target gene. This target gene is subsequently used in methods for identifying compounds that can be used to activate or stimulate osteogenic differentiation, at binding affinity of at most 10 micromolar. Indeed, compounds that are known to bind to target genes used in this screen were found to increase osteogenic differentiation of cells, demonstrating the role of these target genes in this process. This method was used to identify the polypeptides, including the LXR receptor, as involved in the process of osteoblast differentiation, and the use of agonists thereof to promote or induce osteoblast differentiation.

The population of cells, in which osteoblast differentiation is promoted, is preferably any undifferentiated cell type or cell types. Undifferentiated cells are pluripotent cells that are in an early stage of specialization, i.e., which do not yet have their final function and can be induced to form almost any given cell type. Such cells are especially blood cells and cells present in bone marrow, as well as cells derived from adipose tissue. In addition, cells that can still be differentiated into mesenchymal precursor cells are contemplated in the present invention, such as, for example, totipotent stem cells such as embryonic stem cells.

A preferred class of polypeptide used in the knock-in library is in a class of nuclear hormone receptors (NHR). By way of background, lipophilic hormones such as steroids, retinoids, thyroids, and vitamin D₂ modulate gene transcription inside the cell. A steroid hormone, for example, will enter the cell and bind to its complementary receptor, initiating a complex cascade of events. The hormone-receptor complex forms dimers, which bind to a DNA sequence called the hormone response element (HRE). This binding activates, or in some cases inhibits, transcription of the appropriate gene. As such, the activity of NHRs can also be determined with a reporter gene under the control of a promoter that contains the appropriate Hormone Receptor Element (HRE). The most preferred NHR polypeptides identified in Table 1 are NR5A2, NR1H3 and NR1H2 and ESRRG.

Another preferred class of the polypeptides used in the knock-in library ares a G-Protein Coupled Receptors (GPCR), wherein the expression and/or activity of said GPCR may be measured by determining the level of any one of the second messengers cyclic AMP, Ca²⁺ or both. Preferably, the level of the second messenger is determined with a reporter gene under the control of a promoter that is responsive to the second messenger. More preferably, the promoter is a cyclic AMP-responsive promoter, an NF-KB responsive promoter, or a NF-AT responsive promoter. In another preferred embodiment, the reporter gene is selected from the group consisting of: alkaline phosphatase, GFP, eGFP, dGFP, luciferase and b-galactosidase.

One method to measure osteogenic differentiation, and found useful in the screen, determines the expression level of certain proteins that are involved in bone-morphogenesis and that are induced during the differentiation process, such as alkaline phosphatase, type-1 collagen, osteocalcin and osteopontin. The activity levels of these marker proteins can be measured through assays using specific substrates. For instance, the bone alkaline phosphatase (BAP, or bone AP) activity can be measured by adding a methylumbelliferyl heptaphosphate (MUP) solution to the cells. The fluorescence generated upon cleavage of the MUP substrate by the AP activity is measured on a fluorescence plate reader, as outlined in the examples given below. The expression of the target genes can also be determined by methods known in the art such as Western blotting using specific antibodies, or ELISAs using specific antibodies directed against the target genes. Alternatively, one can analyse the mRNA expression levels in cells, using methods known in the art like Northern blotting and quantitative real-time PCR.

Osteogenic differentiation is promoted if the expression or activity of an aforesaid marker protein is induced upon incubation with an agonist compound. Although induction of protein expression levels may vary from an increase of a few percent to two, three or four orders of magnitude higher, induction of protein expression of at least twofold (or more) in a patient (in vivo) is a preferred level. A preferred induction of said expression and/or activity is therefore comparable to an induction of 100% (or more) in vivo. It can however not be excluded that levels found in vitro do not perfectly correlate with levels found in vivo, such that a slightly reduced level in vitro may still result in a higher induction in vivo when the agonist compound is applied in a therapeutic setting. It is therefore preferred to have induced in vitro levels of at least 20%, more preferably more than 50%, even more preferably more than 100%, which would mean a twofold induction of the expression or activity of the osteogenic marker protein.

For screening of a compound that influences the osteogenic differentiation of cells by binding to any of the target polypeptides listed in Table 1, or a derivative, or a fragment thereof, such as the protein domain fragments thereof identified in Table 1A below, libraries of compounds, such as peptide (LOPAP™, Sigma Aldrich), lipid (BioMol), synthetic compound (LOPAC™, Sigma Aldrich) or natural compound (Specs, TimTec) libraries, can be used.

TABLE 1A
Protein Domain Fragments

| | | | Seq ID |
|---|---|---|---|
| | | | protein |
| Accesion | Name | Protein Segment | segment |
| NM_000675 | ADORA2A | Extracellular domain | 201 |
| | | Transmembrane domain | 202 |
| | | Intracellular domain | 203 |
| | | Transmembrane domain | 204 |
| | | Extracellular domain | 205 |
| | | Transmembrane domain | 206 |
| | | Intracellular domain | 207 |
| | | Transmembrane domain | 208 |
| | | Extracellular domain | 209 |
| | | Transmembrane domain | 210 |
| | | Intracellular domain | 211 |
| | | Transmembrane domain | 212 |
| | | Extracellular domain | 213 |
| | | Transmembrane domain | 214 |
| | | Intracellular domain | 215 |
| NM_005684 | GPR52 | Extracellular domain | 216 |
| | | Transmembrane domain | 217 |
| | | Intracellular domain | 218 |
| | | Transmembrane domain | 219 |
| | | Extracellular domain | 220 |
| | | Transmembrane domain | 221 |
| | | Intracellular domain | 222 |
| | | Transmembrane domain | 223 |
| | | Extracellular domain | 224 |
| | | Transmembrane domain | 225 |
| | | Intracellular domain | 226 |
| | | Transmembrane domain | 227 |
| | | Extracellular domain | 228 |
| | | Transmembrane domain | 229 |
| | | Intracellular domain | 230 |
| NM_007369 | GPR161 | Extracellular domain | 231 |
| | | Transmembrane domain | 232 |
| | | Intracellular domain | 233 |
| | | Transmembrane domain | 234 |
| | | Extracellular domain | 235 |
| | | Transmembrane domain | 236 |
| | | Intracellular domain | 237 |
| | | Transmembrane domain | 238 |
| | | Extracellular domain | 239 |
| | | Transmembrane domain | 240 |
| | | Intracellular domain | 241 |
| | | Transmembrane domain | 242 |
| | | Extracellular domain | 243 |
| | | Transmembrane domain | 244 |
| | | Intracellular domain | 245 |
| NM_153832 | GPR161 | Extracellular domain | 246 |
| | | Transmembrane domain | 247 |
| | | Intracellular domain | 248 |
| | | Transmembrane domain | 249 |
| | | Extracellular domain | 250 |
| | | Transmembrane domain | 251 |
| | | Intracellular domain | 252 |
| | | Transmembrane domain | 253 |
| | | Extracellular domain | 254 |
| | | Transmembrane domain | 255 |
| | | Intracellular domain | 256 |
| | | Transmembrane domain | 257 |
| | | Extracellular domain | 258 |
| | | Transmembrane domain | 259 |
| | | Intracellular domain | 260 |
| 3273814CA2 | GPR161 | Extracellular domain | 261 |
| | | Transmembrane domain | 262 |
| | | Intracellular domain | 263 |
| | | Transmembrane domain | 264 |
| | | Extracellular domain | 265 |
| | | Transmembrane domain | 266 |
| | | Intracellular domain | 267 |
| | | Transmembrane domain | 268 |
| | | Extracellular domain | 269 |
| | | Transmembrane domain | 270 |
| | | Intracellular domain | 271 |
| | | Transmembrane domain | 272 |
| | | Extracellular domain | 273 |
| | | Transmembrane domain | 274 |
| | | Intracellular domain | 275 |
| NM_003608 | GPR65 | Extracellular domain | 276 |
| | | Transmembrane domain | 277 |
| | | Intracellular domain | 278 |
| | | Transmembrane domain | 279 |
| | | Extracellular domain | 280 |
| | | Transmembrane domain | 281 |
| | | Intracellular domain | 282 |
| | | Transmembrane domain | 283 |
| | | Extracellular domain | 284 |
| | | Transmembrane domain | 285 |
| | | Intracellular domain | 286 |
| | | Transmembrane domain | 287 |
| | | Extracellular domain | 288 |
| | | Transmembrane domain | 289 |
| | | Intracellular domain | 290 |
| NM_005288 | GPR12 | Extracellular domain | 291 |
| | | Transmembrane domain | 292 |
| | | Intracellular domain | 293 |
| | | Transmembrane domain | 294 |
| | | Extracellular domain | 295 |
| | | Transmembrane domain | 296 |
| | | Intracellular domain | 297 |
| | | Transmembrane domain | 298 |
| | | Extracellular domain | 299 |
| | | Transmembrane domain | 300 |
| | | Intracellular domain | 301 |
| | | Transmembrane domain | 302 |
| | | Extracellular domain | 303 |
| | | Transmembrane domain | 304 |
| | | Intracellular domain | 305 |
| NM_005913 | MC5R | Extracellular domain | 306 |
| | | Transmembrane domain | 307 |
| | | Intracellular domain | 308 |
| | | Transmembrane domain | 309 |
| | | Extracellular domain | 310 |
| | | Transmembrane domain | 311 |
| | | Intracellular domain | 312 |
| | | Transmembrane domain | 313 |
| | | Extracellular domain | 314 |
| | | Transmembrane domain | 315 |
| | | Intracellular domain | 316 |
| | | Transmembrane domain | 317 |
| | | Extracellular domain | 318 |
| | | Transmembrane domain | 319 |
| | | Intracellular domain | 320 |
| NM_000054 | AVPR2 | Extracellular domain | 321 |
| | | Transmembrane domain | 322 |
| | | Intracellular domain | 323 |
| | | Transmembrane domain | 324 |
| | | Extracellular domain | 325 |
| | | Transmembrane domain | 326 |
| | | Intracellular domain | 327 |
| | | Transmembrane domain | 328 |
| | | Extracellular domain | 329 |
| | | Transmembrane domain | 330 |
| | | Intracellular domain | 331 |
| | | Transmembrane domain | 332 |
| | | Extracellular domain | 333 |
| | | Transmembrane domain | 334 |
| | | Intracellular domain | 335 |
| NM_000794 | DRD1 | Extracellular domain | 336 |
| | | Transmembrane domain | 337 |
| | | Intracellular domain | 338 |
| | | Transmembrane domain | 339 |
| | | Extracellular domain | 340 |
| | | Transmembrane domain | 341 |
| | | Intracellular domain | 342 |
| | | Transmembrane domain | 343 |
| | | Extracellular domain | 344 |
| | | Transmembrane domain | 345 |
| | | Intracellular domain | 346 |
| | | Transmembrane domain | 347 |
| | | Extracellular domain | 348 |
| | | Transmembrane domain | 349 |
| | | Intracellular domain | 350 |
| NM_005693 | NR1H3 | Fragments that contain LBD | 351 |
| | | Fragments that contain LBD | 352 |
| | | Fragments that contain LBD | 353 |
| | | Ligand Binding Domain only | 354 |
| U93553- | NR5A2 | Fragments that contain LBD | 355 |
| NM_003822- | | Fragments that contain LBD | 356 |
| NM205860 | | Ligand Binding Domain only | 357 |
| NM_001438- | ESRRG | Fragments that contain LBD | 358 |
| NM_206594- | | Ligand Binding Domain only | 359 |
| NM206595 | | | |
| NM_007121 | NR1H2 | Fragments that contain LBD | 360 |
| | | Fragments that contain LBD | 361 |
| | | Fragments that contain LBD | 362 |
| | | Ligand Binding Domain only | 363 |

The binding affinity of the test compound with the polypeptide can be measured by methods known in the art, such as using surface plasmon resonance biosensors (Biacore), by saturation binding analysis with a labeled compound (e.g. Scatchard and Lindmo analysis), by differential UV spectrophotometer, fluorescence polarization assay, Fluorometric Imaging Plate Reader (FLIPR®) system, Fluorescence resonance energy transfer, and Bioluminescence resonance energy transfer.

The binding affinity of compounds can also be expressed in dissociation constant (Kd) or as IC₅₀ or EC₅₀. The IC₅₀ represents the concentration of a compound that is required for 50% inhibition of binding of another ligand to the polypeptide. The EC₅₀ represents the concentration required for obtaining 50% of the maximum effect in any assay that measures receptor function. The dissociation constant, Kd, is a measure of how well a ligand binds to the polypeptide, it is equivalent to the ligand concentration required to saturate exactly half of the binding-sites on the polypeptide. Compounds with a high binding affinity have low Kd, IC₅₀ and EC₅₀ values, i.e.ᵢₙ the range of 100 nM to 1 pM; a moderate to low affinity binding relates to a high Kd, IC₅₀ and EC₅₀ values, i.e.ᵢₙ the micromolar range. Binding affinities may be determined in in vivo settings as well as in in vitro settings.

The induction of osteogenic differentiation of cells may be achieved in different ways. The compounds found useful in the present invention may target the polypeptides directly and induce or stimulate their activity. These compounds may also target the transcription/translation machinery involved in the transcription and/or translation of the polypeptide from its encoding nucleic acid. The compounds may furthermore target their respective DNAs and mRNAs thereby inducing the occurrence of the polypeptide and thereby their activity. It is thus to be understood that the compounds that are identified by using the methods of the present invention may target the expression, and/or the activity of the polypeptides at different levels, finally resulting in the alteration of the osteogenic differentiation of cells. The agonist compounds of the present invention may function in accordance with any one of these mechanisms.

In Vitro Methods of the Present Invention

A special embodiment of the present invention relates to a method for the in vitro production of bone tissue, comprising applying osteoblast progenitor cells on a substrate, and contacting said cells with an effective osteogenic stimulating amount of an agonist of the target genes identified in Table 1, or an expressible polynucleotide encoding an amino acid sequence of SEQ ID No. 101-118, for a time sufficient to stimulate the generation of bone matrix tissue. More specifically, this method is useful for the in vitro production of bone tissue, by applying mammalian osteoblast progenitor cells on a substrate; adding an agonist of the target genes identified in Table 1, or an expressible polynucleotide encoding an amino acid sequence of SEQ ID No. 101-118; allowing the cells to undergo osteogenic differentiation and to generate bone matrix.

This in vitro produced bone tissue can be used for the provision of load-bearing implants, including joint prostheses, such as artificial hip joints, knee joints and finger joints, and maxillofacial implants, such as dental implants. It can also be used for special surgery devices, such as spacers, or bone fillers, and for use in augmentation, obliteration, or reconstitution of bone defects and damaged or lost bone. The methods of the invention are also very suitable in relation to revision surgery, i.e., when previous surgical devices have to be replaced. A further aspect of this method comprises combining a load-bearing implant (preferably coated with a matrix as described above) with a bone filling composition comprising a matrix as described above.

Preferred cells to use for the in vitro production of bone tissue are undifferentiated cells. Suitable undifferentiated cells are bone marrow cells, including haematopoietic cells and in particular stromal cells. The marrow cells, and especially the stromal cells are found to be very effective in the bone producing process when taken from their original environment. Undifferentiated cells are often available in large quantities, are more conveniently to use than mature bone cells, and exhibit a lower morbidity during recovery. Moreover, the undifferentiated cells can be obtained from the patient for whom the implant is intended. The bone resulting from these cells is autologous to the patient and thus no immune response will be induced.

The undifferentiated cells can be directly applied to the substrate or they can advantageously be multiplied in the absence of the substrate before being applied on the substrate. In the latter mode, the cells are still largely undifferentiated. Subsequently, the cells are allowed to differentiate by adding at least one of the expressible polynucleotides as described herein, or an agonist for one or more of the target genes of Table 1, which agonist is known in the art or which has been identified using any of the methods described herein.

Bone formation can be optimized by variation in mineralization, both by inductive and by conductive processes. In this way, matrices up to 100 μm in thickness can be produced. The cells are cultured for a time sufficient to produce a matrix layer, for example, a matrix layer having a thickness of at least 0.5 micrometer (μm), preferably between 1 and 100 μm, and more preferably between 10 and 50 μm. The cells may be contacted with the culture medium for any length of time.

The production of the matrix, when applied on a substrate, results in a continuous or quasi-continuous coating covering the substrate for at least 50% of its surface area. The substrate on which the undifferentiated cells can be applied and cultured can be a metal, such as titanium, cobalt/chromium alloy or stainless steel, a bioactive surface such as a calcium phosphate, polymer surfaces such as polyethylene, and the like.

In another embodiment, the present invention relates to cells that have undergone osteoblast differentiation by treatment with compounds as disclosed herein and identifiable according to any one of the methods described herein.

Methods of Therapy and Pharmaceutical Compositions

The present inventors discovered that the polypeptides listed in Table 1 are involved in the osteogenic differentiation process. Accordingly, the present invention relates to the link between certain polypeptides present in the cell with osteogenic differentiation of cells, some of which are closely related to the onset, occurrence, and substantiation of metabolic bone diseases. Accordingly, the present invention relates not only to the compounds that may be used for targeting these polypeptides (many of which are known in the art) but also to the use of such compounds for therapeutic purposes related to diseases of bone metabolism. For the compounds that are already known to bind to these polypeptides, the use thereof in the present invention is a new (medical) use.

A preferred aspect of the present invention relates to a method for the treatment or prevention of an imbalance in bone homeostasis comprising administering an effective osteogenic stimulating amount of an agonist of one or more of the target genes identified in Table 1, or an expressible polynucleotide encoding one or more of an amino acid sequence of SEQ ID No. 101-118, to a subject suffering from or susceptible to said imbalance. Such imbalance is characterized by a reduction in the ratio of osteoblasts to osteoclasts in the bone tissue of a subject. More particularly, this reduction is in the ratio of osteoblasts that are effective in mineralizing the bone matrix relative to the osteoclasts effectively resorbing bone minerals, specifically calcium.

The present method is useful for the treatment of subjects susceptible to or suffering from hypocalcaemia (of malignancy), Paget's disease, rheumatoid arthritis, periodontal disease, focal osteogenesis occurring during skeletal metastases, Crouzon's syndrome, rickets, opsismodysplasia, pycnodysostosis/Toulouse-Lautrec disease, osteogenesis imperfecta and/or osteoporosis.

Administering of the target gene agonist or expressible polynucleic acid encoding said target gene to the subject patient includes both self-administration and administration by another person. The patient may be in need of treatment for an existing disease or medical condition, or may desire prophylactic treatment to prevent or reduce the risk for diseases and medical conditions affected by a disturbance in bone metabolism. The osteogenic differentiation medicament may be delivered to the subject patient orally, transdermally, via inhalation, injection, nasally, rectally, or via a sustained release formulation.

The polynucleotide expressing the osteogenic differentiation agent comprising an expressible polynucleic acid encoding one or more of polypeptides of SEQ ID NO 101-118 is preferably included within a vector. The polynucleic acid is operably linked to signals enabling expression of the nucleic acid sequence and is introduced into a cell utilizing, preferably, recombinant vector constructs, which will express the antisense nucleic acid once the vector is introduced into the cell. A variety of viral-based systems are available, including adenoviral, retroviral, adeno-associated viral, lentiviral, herpes simplex viral or a sendaviral vector systems, and all may be used to introduce and express polynucleotide sequence for the osteogenic differentiation polypeptides of SEQ ID NO. 101-118 in target cells.

Preferably, the viral vectors used in the methods of the present invention are replication defective. Such replication defective vectors will usually pack at least one region that is necessary for the replication of the virus in the infected cell. These regions can either be eliminated (in whole or in part), or be rendered non-functional by any technique known to a person skilled in the art. These techniques include the total removal, substitution, partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome, which are necessary for encapsidating, the viral particles.

In a preferred embodiment, the viral element is derived from an adenovirus. Preferably, the vehicle includes an adenoviral vector packaged into an adenoviral capsid, or a functional part, derivative, and/or analogue thereof. Adenovirus biology is also comparatively well known on the molecular level. Many tools for adenoviral vectors have been and continue to be developed, thus making an adenoviral capsid a preferred vehicle for incorporating in a library of the invention. An adenovirus is capable of infecting a wide variety of cells. However, different adenoviral serotypes have different preferences for cells. To combine and widen the target cell population that an adenoviral capsid of the invention can enter in a preferred embodiment, the vehicle includes adenoviral fiber proteins from at least two adenoviruses. Preferred adenoviral fiber protein sequences are serotype 17, 45 and 51. Techniques or construction and expression of these chimeric vectors are disclosed in US Published Patent Applications 20030180258 and 20040071660, hereby incorporated by reference.

In a preferred embodiment, the nucleic acid derived from an adenovirus includes the nucleic acid encoding an adenoviral late protein or a functional part, derivative, and/or analogue thereof. An adenoviral late protein, for instance an adenoviral fiber protein, may be favorably used to target the vehicle to a certain cell or to induce enhanced delivery of the vehicle to the cell. Preferably, the nucleic acid derived from an adenovirus encodes for essentially all adenoviral late proteins, enabling the formation of entire adenoviral capsids or functional parts, analogues, and/or derivatives thereof. Preferably, the nucleic acid derived from an adenovirus includes the nucleic acid encoding adenovirus E2A or a functional part, derivative, and/or analogue thereof. Preferably, the nucleic acid derived from an adenovirus includes the nucleic acid encoding at least one E4-region protein or a functional part, derivative, and/or analogue thereof, which facilitates, at least in part, replication of an adenoviral derived nucleic acid in a cell. The adenoviral vectors used in the examples of this application are exemplary of the vectors useful in the present method of treatment invention.

Certain embodiments of the present invention use retroviral vector systems. Retroviruses are integrating viruses that infect dividing cells, and their construction is known in the art. Retroviral vectors can be constructed from different types of retrovirus, such as, MoMuLV (“murine Moloney leukemia virus” MSV (“murine Moloney sarcoma virus”), HaSV (“Harvey sarcoma virus”); SNV (“spleen necrosis virus”); RSV (“Rous sarcoma virus”) and Friend virus. Lentiviral vector systems may also be used in the practice of the present invention.

In other embodiments of the present invention, adeno-associated viruses (“AAV”) are utilized. The AAV viruses are DNA viruses of relatively small size that integrate, in a stable and site-specific manner, into the genome of the infected cells. They are able to infect a wide spectrum of cells without inducing any effects on cellular growth, morphology or differentiation, and they do not appear to be involved in human pathologies.

In the vector construction, the polynucleotide agents of the present invention may be linked to one or more regulatory regions. Selection of the appropriate regulatory region or regions is a routine matter, within the level of ordinary skill in the art. Regulatory regions include promoters, and may include enhancers, suppressors, etc.

Promoters that may be used in the expression vectors of the present invention include both constitutive promoters and regulated (inducible) promoters. The promoters may be prokaryotic or eukaryotic depending on the host. Among the prokaryotic (including bacteriophage) promoters useful for practice of this invention are lac, lacZ, T3, T7, lambda Pᵣ, Pₗ, and trp promoters. Among the eukaryotic (including viral) promoters useful for practice of this invention are ubiquitous promoters (e.g. HPRT, vimentin, actin, tubulin), intermediate filament promoters (e.g. desmin, neurofilaments, keratin, GFAP), therapeutic gene promoters (e.g. MDR type, CFTR, factor VIII), tissue-specific promoters (e.g. actin promoter in smooth muscle cells, or Flt and Flk promoters active in endothelial cells), including animal transcriptional control regions, which exhibit tissue specificity and have been utilized in transgenic animals: elastase I gene control region which is active in pancreatic acinar cells (Swift, et al. (1984) Cell 38:639-46; Ornitz, et al. (1986) Cold Spring Harbor Symp. Quant. Biol. 50:399-409; MacDonald, (1987) Hepatology 7:425-515); insulin gene control region which is active in pancreatic beta cells (Hanahan, (1985) Nature 315:115-22), immunoglobulin gene control region which is active in lymphoid cells (Grosschedl, et al. (1984) Cell 38:647-58; Adames, et al. (1985) Nature 318:533-8; Alexander, et al. (1987) Mol. Cell. Biol. 7:1436-44), mouse mammary tumor virus control region which is active in testicular, breast, lymphoid and mast cells (Leder, et al. (1986) Cell 45:485-95), albumin gene control region which is active in liver (Pinkert, et al. (1987) Genes and Devel. 1:268-76), alpha-fetoprotein gene control region which is active in liver (Krumlauf, et al. (1985) Mol. Cell. Biol., 5:1639-48; Hammer, et al. (1987) Science 235:53-8), alpha 1-antitrypsin gene control region which is active in the liver (Kelsey, et al. (1987) Genes and Devel., 1: 161-71), beta-globin gene control region which is active in myeloid cells (Mogram, et al. (1985) Nature 315:338-40; Kollias, et al. (1986) Cell 46:89-94), myelin basic protein gene control region which is active in oligodendrocyte cells in the brain (Readhead, et al. (1987) Cell 48:703-12), myosin light chain-2 gene control region which is active in skeletal muscle (Sani, (1985) Nature 314.283-6), and gonadotropic releasing hormone gene control region which is active in the hypothalamus (Mason, et al. (1986) Science 234:1372-8).

Other promoters which may be used in the practice of the invention include promoters which are preferentially activated in dividing cells, promoters which respond to a stimulus (e.g. steroid hormone receptor, retinoic acid receptor), tetracycline-regulated transcriptional modulators, cytomegalovirus immediate-early, retroviral LTR, metallothionein, SV-40, E1a, and MLP promoters.

Additional vector systems include the non-viral systems that facilitate introduction of polynucleotide agents into a patient. For example, a DNA vector encoding a desired sequence can be introduced in vivo by lipofection. Synthetic cationic lipids designed to limit the difficulties encountered with liposome-mediated transfection can be used to prepare liposomes for in vivo transfection of a gene encoding a marker (Feigner, et. al. (1987) Proc. Natl. Acad Sci. USA 84:7413-7); see Mackey, et al. (1988) Proc. Natl. Acad. Sci. USA 85:8027-31; Ulmer, et al. (1993) Science 259:1745-8). The use of cationic lipids may promote encapsulation of negatively charged nucleic acids, and also promote fusion with negatively charged cell membranes (Felgner and Ringold, (1989) Nature 337:387-8). Particularly useful lipid compounds and compositions for transfer of nucleic acids are described in International Patent Publications WO 95/18863 and WO 96/17823, and in U.S. Pat. No. 5,459,127. The use of lipofection to introduce exogenous genes into the specific organs in vivo has certain practical advantages and directing transfection to particular cell types would be particularly advantageous in a tissue with cellular heterogeneity, for example, pancreas, liver, kidney, and the brain. Lipids may be chemically coupled to other molecules for the purpose of targeting. Targeted peptides, e.g., hormones or neurotransmitters, and proteins for example, antibodies, or non-peptide molecules could be coupled to liposomes chemically. Other molecules are also useful for facilitating transfection of a nucleic acid in vivo, for example, a cationic oligopeptide (e.g., International Patent Publication WO 95/21931), peptides derived from DNA binding proteins (e.g., International Patent Publication WO 96/25508), or a cationic polymer (e.g., International Patent Publication WO 95/21931).

It is also possible to introduce a DNA vector in vivo as a naked DNA plasmid (see U.S. Pat. Nos. 5,693,622, 5,589,466 and 5,580,859). Naked DNA vectors for therapeutic purposes can be introduced into the desired host cells by methods known in the art, e.g., transfection, electroporation, microinjection, transduction, cell fusion, DEAE dextran, calcium phosphate precipitation, use of a gene gun, or use of a DNA vector transporter (see, e.g., Wilson, et al. (1992) J. Biol. Chem. 267:963-7; Wu and Wu, (1988) J. Biol. Chem. 263:14621-4; Hartmut, et al. Canadian Patent Application No. 2,012,311, filed Mar. 15, 1990; Williams, et al (1991). Proc. Natl. Acad. Sci. USA 88:2726-30). Receptor-mediated DNA delivery approaches can also be used (Curiel, et al. (1992) Hum. Gene Ther. 3:147-54; Wu and Wu, (1987) J. Biol. Chem. 262:4429-32).

The present invention also provides biologically compatible, osteogenic differentiation compositions comprising an effective amount of one or more compounds identified as target agonists, and/or the osteogenic differentiation polynucleic acids encoding polyeptides of SEQ ID NOs. 101-118 as described hereinabove.

A biologically compatible composition is a composition, that may be solid, liquid, gel, or other form, in which the compound, polynucleotide, vector, and antibody of the invention is maintained in an active form, e.g., in a form able to effect a biological activity. For example, a compound of the invention would have inverse agonist or antagonist activity on the target; a nucleic acid would be able to replicate, translate a message, or hybridize to a complementary mRNA of a target; a vector would be able to transfect a target cell and expression the antisense, antibody, ribozyme or siRNA as described hereinabove; an antibody would bind a target polypeptide domain.

A preferred biologically compatible composition is an aqueous solution that is buffered using, e.g., Tris, phosphate, or HEPES buffer, containing salt ions. Usually the concentration of salt ions will be similar to physiological levels. Biologically compatible solutions may include stabilizing agents and preservatives. In a more preferred embodiment, the biocompatible composition is a pharmaceutically acceptable composition. Such compositions can be formulated for administration by topical, oral, parenteral, intranasal, subcutaneous, and intraocular, routes. Parenteral administration is meant to include intravenous injection, intramuscular injection, intraarterial injection or infusion techniques. The composition may be administered parenterally in dosage unit formulations containing standard, well-known non-toxic physiologically acceptable carriers, adjuvants and vehicles as desired.

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient. Pharmaceutical compositions for oral use can be prepared by combining active compounds with solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropylmethyl-cellulose, or sodium carboxymethyl-cellulose; gums including arabic and tragacanth; and proteins such as gelatin and collagen. If desired, disintegrating or solubilizing agents may be added, such as the cross-linked polyvinyl pyrrolidone, agar, alginic acid, or a salt thereof, such as sodium alginate. Dragee cores may be used in conjunction with suitable coatings, such as concentrated sugar solutions, which may also contain gum arabic, talc, polyvinyl-pyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Pharmaceutical preparations that can be used orally include push-fit capsules made of gelatin, as well as soft, sealed capsules made of gelatin and a coating, such as glycerol or sorbitol. Push-fit capsules can contain active ingredients mixed with filler or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Preferred sterile injectable preparations can be a solution or suspension in a non-toxic parenterally acceptable solvent or diluent. Examples of pharmaceutically acceptable carriers are saline, buffered saline, isotonic saline (e.g. monosodium or disodium phosphate, sodium, potassium; calcium or magnesium chloride, or mixtures of such salts), Ringer's solution, dextrose, water, sterile water, glycerol, ethanol, and combinations thereof 1,3-butanediol and sterile fixed oils are conveniently employed as solvents or suspending media. Any bland fixed oil can be employed including synthetic mono- or di-glycerides. Fatty acids such as oleic acid also find use in the preparation of injectables.

The composition medium can also be a hydrogel, which is prepared from any biocompatible or non-cytotoxic homo- or hetero-polymer, such as a hydrophilic polyacrylic acid polymer that can act as a drug absorbing sponge. Certain of them, such as, in particular, those obtained from ethylene and/or propylene oxide are commercially available. A hydrogel can be deposited directly onto the surface of the tissue to be treated, for example during surgical intervention.

Embodiments of pharmaceutical compositions of the present invention comprise a replication defective recombinant viral vector encoding the polynucleotide of SEQ ID NOs. 1-18 of the present invention and a transfection enhancer, such as poloxamer. An example of a poloxamer is Poloxamer 407, which is commercially available (BASF, Parsippany, N.J.) and is a non-toxic, biocompatible polyol. A poloxamer impregnated with recombinant viruses may be deposited directly on the surface of the tissue to be treated, for example during a surgical intervention. Poloxamer possesses essentially the same advantages as hydrogel while having a lower viscosity.

The active osteogenic differentiating agents (whether they are vectors encoding target genes or small molecule agonists) may also be entrapped in microcapsules prepared, for example, by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (1980) 16th edition, Osol, A. Ed.

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™, (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

As defined above, therapeutically effective dose means that amount of protein, polynucleotide, peptide, or its antibodies, agonists or antagonists, which ameliorate the symptoms or condition. Therapeutic efficacy and toxicity of such compounds can be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions that exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

For any compound, the therapeutically effective dose can be estimated initially either in cell culture assays or in animal models, usually mice, rabbits, dogs, or pigs. The animal model is also used to achieve a desirable concentration range and route of administration. Such information can then be used to determine useful doses and routes for administration in humans. The exact dosage is chosen by the individual physician in view of the patient to be treated. Dosage and administration are adjusted to provide sufficient levels of the active moiety or to maintain the desired effect. Additional factors which may be taken into account include the severity of the disease state, age, weight and gender of the patient; diet, desired duration of treatment, method of administration, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

The pharmaceutical compositions according to this invention may be administered to a subject by a variety of methods. They may be added directly to target tissues, complexed with cationic lipids, packaged within liposomes, or delivered to target cells by other methods known in the art. Localized administration to the desired tissues may be done by direct injection, transdermal absorption, catheter, infusion pump or stent. The DNA, DNA/vehicle complexes, or the recombinant virus particles are locally administered to the site of treatment. Alternative routes of delivery include, but are not limited to, intravenous injection, intramuscular injection, subcutaneous injection, aerosol inhalation, oral (tablet or pill form), topical, systemic, ocular, intraperitoneal and/or intrathecal delivery. Examples of ribozyme delivery and administration are provided in Sullivan et al. WO 94/02595.

As discussed hereinabove, recombinant viruses may be used to introduce DNA encoding polynucleotide agents useful in the present invention. Recombinant viruses according to the invention are generally formulated and administered in the form of doses of between about 10⁴ and about 10¹⁴ pfu. In the case of AAVs and adenoviruses, doses of from about 10⁶ to about 10¹¹ pfu are preferably used. The term pfu (“plaque-forming unit”) corresponds to the infective power of a suspension of virions and is determined by infecting an appropriate cell culture and measuring the number of plaques formed. The techniques for determining the pfu titre of a viral solution are well documented in the prior art.

The present invention also provides methods of enhancing bone formation, which comprise the administration to said subject of a therapeutically effective amount of an osteogenic differentiating agent of the invention. A further aspect of the invention relates to a method of treating or preventing a disease involving an imbalance in bone homeostasis, comprising administering to said subject an osteogenic differentiating pharmaceutical composition as described herein.

The polypeptides or the polynucleotides employed in the methods of the present invention may be free in solution, affixed to a solid support, borne on a cell surface, or located intracellularly. To perform the methods it is feasible to immobilize either the polypeptide identified in Tables 1 and/or 1A or the compound to facilitate separation of complexes from uncomplexed forms of the polypeptide, as well as to accommodate automation of the assay. Interaction (e.g., binding of) of the polypeptide of the present invention with a compound can be accomplished in any vessel suitable for containing the reactants. Examples of such vessels include microtitre plates, test tubes, and microcentrifuge tubes. In one embodiment, a fusion protein can be provided which adds a domain that allows the polypeptide to be bound to a matrix. For example, the polypeptide of the present invention can be “His” tagged, and subsequently adsorbed onto Ni-NTA microtitre plates, or ProtA fusions with the polypeptides of the present invention can be adsorbed to IgG, which are then combined with the cell lysates (e.g., (³⁵S-labelled) and the candidate compound, and the mixture incubated under conditions favorable for complex formation (e.g., at physiological conditions for salt and pH). Following incubation, the plates are washed to remove any unbound label, and the matrix is immobilized. The amount of radioactivity can be determined directly, or in the supernatant after dissociation of the complexes. Alternatively, the complexes can be dissociated from the matrix, separated by SDS-PAGE, and the level of the protein binding to the protein of the present invention quantitated from the gel using standard electrophoretic techniques.

Other techniques for immobilizing protein on matrices can also be used in the method of identifying compounds. For example, either the polypeptide or the compound can be immobilized utilizing conjugation of biotin and streptavidin. Biotinylated protein molecules of the present invention can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with the polypeptides of the present invention but which do not interfere with binding of the polypeptide to the compound can be derivatized to the wells of the plate, and the polypeptide of the present invention can be trapped in the wells by antibody conjugation. As described above, preparations of a labeled candidate compound are incubated in the wells of the plate presenting the polypeptide of the present invention, and the amount of complex trapped in the well can be quantitated.

### Detailed Experimental Study Linking Target Gene Agonists and Osteoblast Differentiation

### EXAMPLE 1

### Screening of FLeXSelect Libraries for Modulators of Endogenous Alkaline Phosphatase in Primary Human MPCs

Materials:

Adenoviral constructs:

Ad-BMP2: Described in WO 03/018799

Ad-eGFP: Referred to as pIPspAdApt6-EGFP in WO 02070744

Ad-LacZ: Referred to as pIPspAdApt6-lacZ in WO 02070744

Ad-empty: Referred to as empty virus (generated from pIPspAdApt 6) in WO 02070744

Ad-hCAR: hCAR cDNA is isolated using a PCR methodology. The following hCAR-specific primers are used: HuCAR_for 5′-GCGAAGCTTCCATGGCGCTCCTGCTGTGCTTCG-3′ and HuCAR_rev 5′-GCGGGATCCATCTATACTATAGACCCATCCTTGCTC-3′. The hCAR cDNA is PCR amplified from a HeLa cell cDNA library (Quick clone, Clontech). A single fragment of 1119 bp is obtained and digested with the HindIII and BamHI restriction enzymes. pIPspAdapt6 vector (WO99/64582) is digested with the same enzymes, gel-purified and used to ligate to the digested PCR hCAR fragment. AdC20 (Ad5/Ad51) viruses are generated as described in WO02/24933

H4-2: described as DLL4_v1 in WO03/018799

H4-291: SPINT1_v1. cDNA is prepared from RNA isolated from human placenta and cloned in the pIPspAdapt 6 plasmid using SalI-NotI restriction sites as described in WO02/070744. The protein encoded by H4-291 is identical to NP_{—}003701.

Principle of the Assay

Mesenchymal progenitor cells (MPCs) differentiate into osteoblasts in the presence of appropriate factors (e.g. BMP2). An assay to screen for such factors is developed by monitoring the activity of alkaline phosphatase (AP) enzyme, an early marker in the osteoblast differentiation program. MPCs are seeded in 384 well plates and simultaneously co-infected one day later with adenoviruses encoding the human coxsackie and adenovirus receptor (hCAR; Ad-hCAR) and individual adenoviruses (Ad-cDNA) from the arrayed adenoviral knock-in collection containing cDNA sequences corresponding to genes from “drugable” classes like GPCR's, kinases, proteases, phosphodiesterases and nuclear hormone receptors (the FLeXSelect collection). The majority of these cDNAs are obtained by a PCR-based approach. Briefly, PCR primers are designed for amplification of the complete open reading frame from ATG start codon to the stop codon of drugable genes, based on sequence data present in the RefSeq database. Primers are mixed in an arrayed format at a PCR ready concentration in 96 well plates. As a template for the PCR reactions, placental, fetal liver, fetal brain and spinal cord cDNA libraries are used (from Invitrogen or Edge Biosystems). For the genes encoded by a single exon, PCR reactions are also performed on human genomic DNA. After the amplification reactions, the PCR products are purified with a 96-well PCR clean-up system (Wizard magnesil, Promega, Madison, Wis., USA), digested with the appropriate restriction enzymes (AscI, NotI or SalI restriction sites are included in the primers) and directly cloned into the adenoviral adapter plasmid plspAdAdapt-10-Zeo (described in U.S. Pat. No. 6,340,595) using DNA ligation kit version 2 (TaKaRa, Berkeley, Calif., USA). After a transformation and selection step, multiple clones per gene, one of which is sequence verified, are used for the preparation of plasmid DNA and subsequent generation of adenovirus according to the procedure described in WO99/64582.

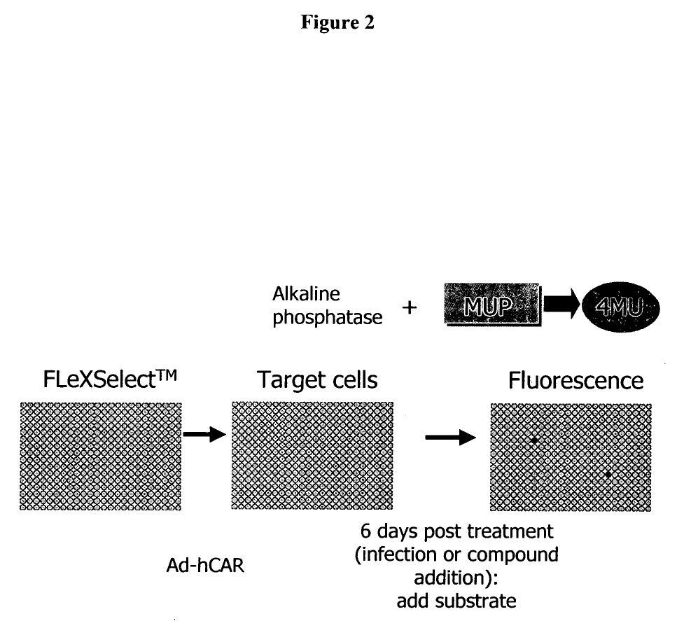

It is found that co-infection with AdC20-hCAR (MOI 250) increases the AdC01-cDNA infection efficiency. Cellular AP activity is determined 6 days after the infection (or ligand addition—see below). The principle of the assays is depicted in FIG. 2. Mesenchymal stem cells derived from bone marrow are infected with the FLeXSelect™ cDNA library viruses in the presence of Ad5C15-hCAR or Ad5C20-hCAR virus. Six days after the start of infection or treatment with a ligand, endogenous alkaline phosphatase activity is measured following addition of 4-methylumbelliferyl heptaphosphate (MUP) substrate.

Development of the Assay

MPCs are isolated from bone marrow of healthy volunteers, obtained after informed consent (Cambrex/Biowhittaker, Verviers, Belgium).

In a series of experiments carried out in 384 well plates, several parameters are optimized: cell seeding density, multiplicities of infection (MOI) of control viruses (Ad-BMP2 or Ad-eGFP), MOI of Ad-hCAR, duration of infection, toxicity, infection efficiency (using Ad-eGFP) and the day of readout.

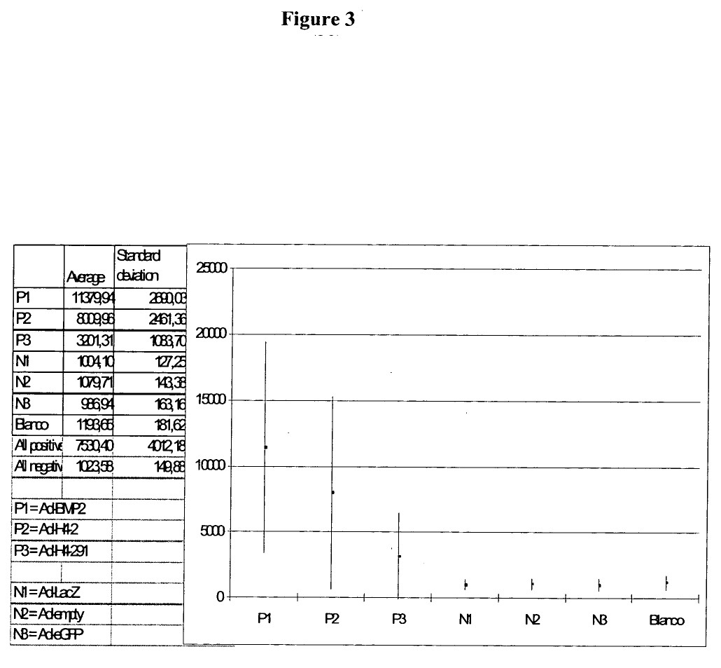

The following protocol resulted in the highest dynamic range for the assay with the lowest standard deviation on the background signal: MPCs are seeded on day 0 at 1000 cells per well of a 384 well plate and co-infected the next day using a mix of AdC20-hCAR and 2 μl of Ad-control-viruses. The stocks of the Ad-control-viruses are generated in 96 well plates (control plate). The 2 μl volume corresponds to a theoretical MOI of 5000. Controls are: P1=Ad-BMP2; P2=Ad-H4-2; P3=Ad-H4-291; N1=Ad-LacZ; N2=Ad-empty; N3=Ad-eGFP. Up-regulation of alkaline phosphatase is read at 6 days post infection (6 dpi): 15 μl 4-Methylumbelliferyl-phosphate (MUP, Sigma) is added to each well, the plates are incubated for 15 min at 37° C. and monitored for AP activity using a fluorescence plate reader (Fluostar, BMG). Pipetting of viruses from 96 well plates (containing control viruses) or 384 well plates (containing FleXSelect viruses (see next paragraph)) into 384 well plates containing MPCs is performed using robotics (96/384 channel dispensor Tecan Freedom 200 equipped with TeMO96, TeMO384 and RoMa, Tecan AG, Switzerland). FIG. 3 shows results of the automated screening procedure using the control plate. The mean and standard deviations of the negative controls (N1-N3) are used to calculate a cut-off for hit analysis. The positive controls (P1, P2, P3) routinely scored in 80-100% of the infected wells (FIG. 3). The negative control viruses routinely scored in 0-5% of the infected wells (FIG. 3).

FleXSelect Libraries

Galapagos Genomics NV (Galapagos) built proprietary knock-in (FLeXSelect) arrayed adenoviral libraries encoding most of the drugable genes present in the human genome. The alkaline phosphatase assay is useful to screen viruses from the FLeXSelect collection (Ad-cDNA) for those classes of drugable targets that can be activated by a compound, e.g. G-protein coupled receptors (GPCRs) and nuclear hormone receptors (NHRs).

For a subset of the Ad-GPCRs present in the FLeXSelect library a matching collection of ligands is prepared in 96 and 384 well plates, such that robotics can be used to pipet a matching pair of Ad-GPCR and ligand from the respective stocks in one well of a 384 well plate containing MPCs.

Screening

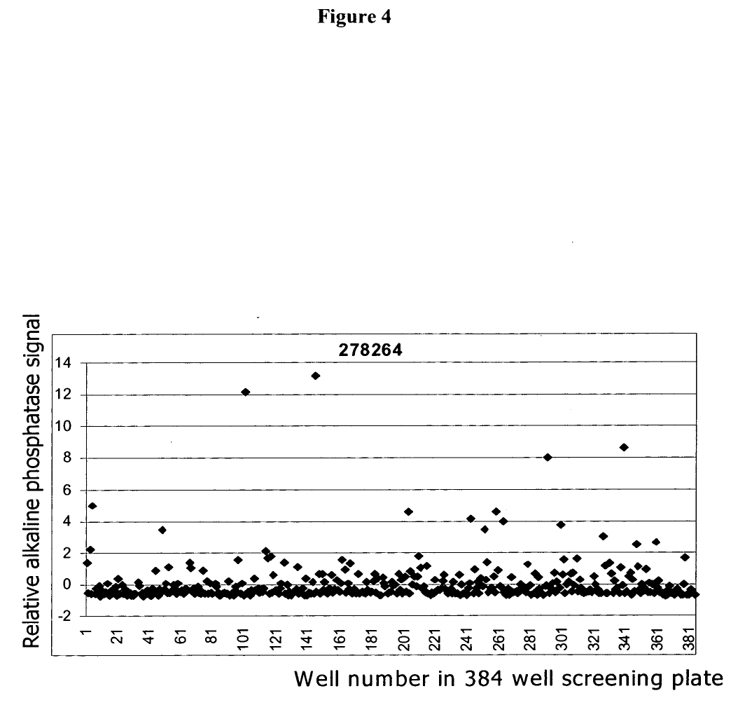

The FLeXSelect viruses, in the presence or absence of matching ligands, are screened according to the protocol described above in duplicate in two independent screens, with each singular sample added on a different plate. If ligands are included in the screening, the protocol is modified: the Ad-cDNA infection is carried out on Day 1, ligands are added on Day 2 and endogenous BAP levels are measured on Day 8. A typical result of a 384 well screening plate is depicted in FIG. 4. Indicated in FIG. 4 are the positions in the 384 well plate on the X-axis and relative alkaline phosphatase signals on the Y-axis. The relative alkaline phosphatase signal for a given sample is calculated as the number of standard deviations above the mean for all data points in a given batch (or experiment).

### EXAMPLE 2

### Target Identification Using the AP Assay

Targets are selected according to the following selection criteria:

1) AP signals higher than the mean plus 3 times the standard deviation of all samples (data points) in the batch. The two individual data points within each batch are analyzed independently.

2) Positive AP signals, as defined by criterion 1, for at least two of the four or 3 of the four virus samples that are screened in duplicate in two independent experiments (total of 4 measurements per virus).

Table 1 lists the targets identified according to the above criteria in the alkaline phosphatase assay.

For some of the targets, agonist ligands are known. These can be used to validate the osteogenic potential of the target genes in MPCs: addition of increasing concentrations of ligand to the medium of MPCs (over-expressing the target protein) should dose-dependently increase the up-regulation of the endogenous alkaline phosphatase activity. This is for example observed when MPCs are infected with Ad-NR1H3 and treated with T0901317, and when MPCs are infected with Ad-GPR65 and treated with 1-b-D-Galactosylsphingosine, and when MPCs are infected with Ad-AVPR2 and treated with [deamino-Cys1,D-Arg8]-Vasopressin.

Ad-NR1H3 and T0901317

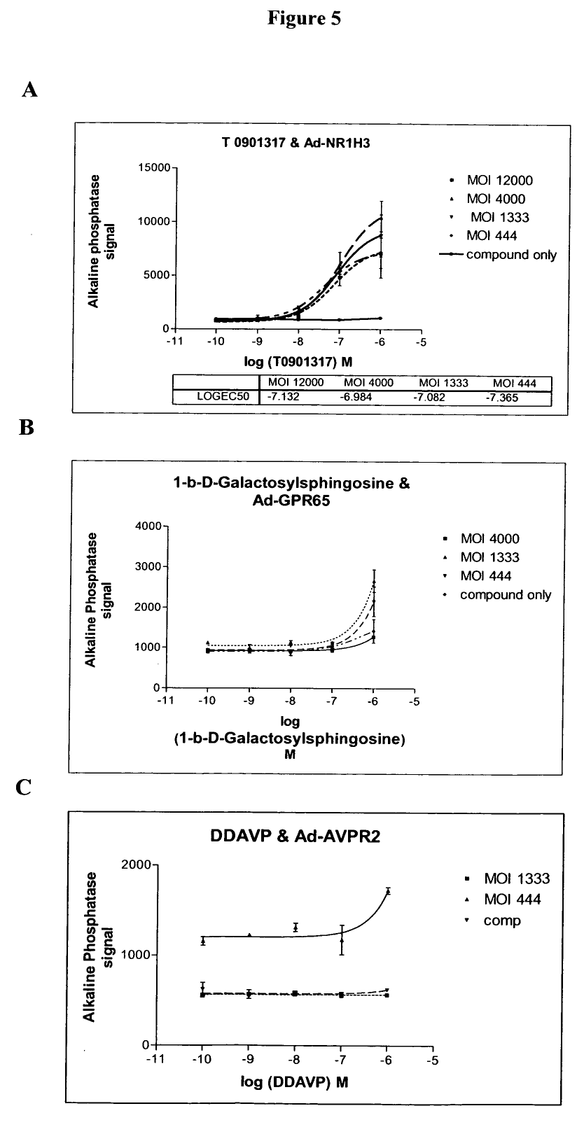

These dose-response curves are depicted in FIG. 5. A dose-response curve for AP activity is generated for MPCs infected with Ad-NR1H3 and treated with T0901317 (FIG. 5A). MPCs are seeded on day 0 at 1000 cells per well of a 384 well plate and co-infected the next day using AdC51-hCAR (MOI 250) and different MOIs of Ad5-NR1H3 (MOI 12000, 4000, 1333, 444). On day 1, 5 concentrations (1E-10M, 1E-9M, 1E-8M, 1E-7M, 1E-6M) of the compound T0901317 (Cayman Chemical, Michigan USA, Cat. No. 71810) with fixed vehicle concentration (the vehicle is DMSO at the concentration is 0.01 %) are added to the wells. After incubation for 6 days at 37° C., 10% CO2 in a humidified incubator, up-regulation of alkaline phosphatase is read: 15 μl MUP is added to each well, the plates are incubated for 15 min at 37° C. and monitored for AP activity using a fluorescence plate reader (Fluostar, BMG).

Dose-response curves for AP activity are generated in a similar way for MPCs infected with Ad-GPR65 and treated with 1-b-D-Galactosylsphingosine (FIG. 5B); for MPCs infected with Ad-AVPR2 and treated with [deamino-Cys1, D-Arg8]-Vasopressin (DDAVP) (FIG. 5C). Three targets are identified that show a dose-dependent up-regulation of AP activity in the AP assay, when the respective ligands are added at different concentrations.

AdNR1H3 and GW3965

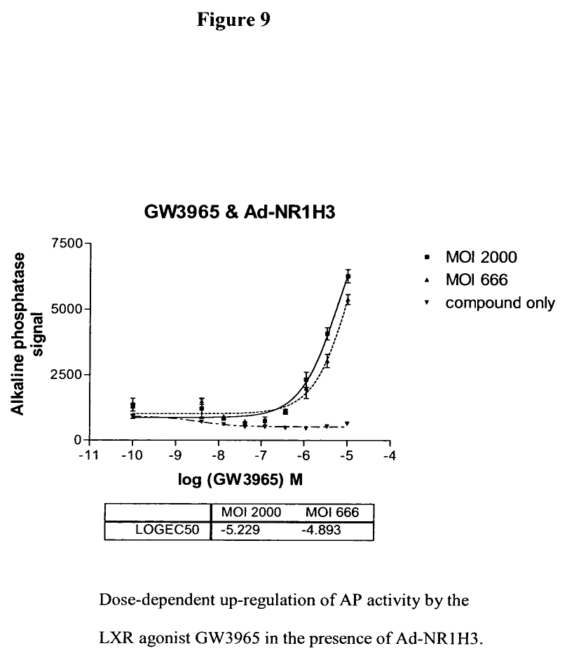

A dose-response relation is observed for AP activity when MPCs are infected with Ad-NR1H3 and treated with GW3965 (FIG. 9). MPCs are seeded on day 0 at 1000 cells per well of a 384 well plate and co-infected the next day using AdC51-hCAR (MOI 250) and different MOIs of Ad5-NR1H3 (MOI 2000, 666). On day 1, 8 concentrations (3,43E-9M, 1,34E-8M, 5,35E-8M, 1,60E-7M, 4,81E-7M, 1,43E-6M; 4,29E-6M, 13E-6M) of the compound GW3965 (Chemovation, West Sussex) with fixed vehicle concentration (DMSO at final concentration of 0.1%) are added to the wells. After 6 days, medium is removed and replaced with fresh medium containing the same concentrations of the compound GW3965. Readouts of AP activity are performed at several time points after the start of the experiment, typically after 7, 10 and 13 days. Up-regulation of alkaline phosphatase activity is read as follows: medium is removed from the mono-layers, 15 μl MUP is added to each well, the plates are incubated for 15 min at 37° C. and then read for AP activity using a fluorescence plate reader (Fluostar, BMG). FIG. 9 illustrates the dose-response activity of GW3965 in the presence of Ad-NR1H3.

AdNR1H2 and T0901317

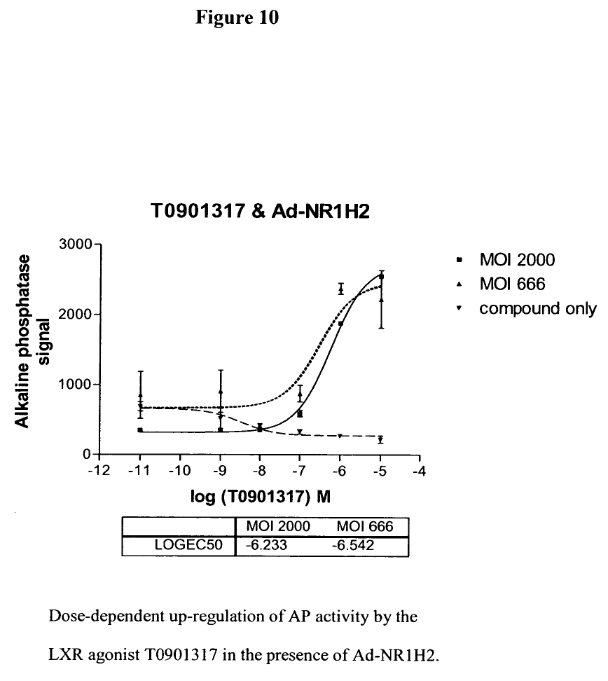

A dose-response relation is observed for AP activity when MPCs are infected with Ad-NR1H2 and treated with T0901317 (FIG. 10). MPCs are seeded on day 0 at 1000 cells per well of a 384 well plate and co-infected the next day using AdC51-hCAR (MOI 250) and different MOIs of Ad5-NR1H3 (MOI 2000,666). On day 1, 5 concentrations (1E-9M, 1E-8M, 1E-7M, 1E-6M, 1E-5M) of the compound T0901317 (Cayman Chemical, Michigan USA, Cat. No. 71810) with fixed vehicle concentration (DMSO at final concentration of 0.1%) are added to the wells. After 6 days, medium is removed and replaced with fresh medium containing the same concentrations of the compound T0901317. Readouts of AP activity are performed at several time points after the start of the experiment, typically after 7, 10 and 13 days. Up-regulation of alkaline phosphatase activity is read as follows: medium is removed from the monolayers, 15 μl MUP is added to each well, the plates are incubated for 15 min at 37° C. and then read for AP activity using a fluorescence plate reader (Fluostar, BMG). FIG. 10 illustrates the dose-response activity of T0901317 in the presence of Ad-NR1H2.

In conclusion, AP activity is up-regulated in cells transduced with either NR1H3 and NR1H2 in a dose-dependent manner when LXR agonists, GW3965 and T0901317, respectively, are added to the cells at different concentrations in the AP assay.

### EXAMPLE 3

### mRNA and Protein Expression Analysis for the Identified Targets

The assay presented in Example 1 demonstrates the discovery of proteins with osteogenic potential upon overexpression. In order to confirm that these proteins are endogenously expressed in bone forming cells such as MPCs or primary human osteoblasts (hOBs), mRNA is extracted from these cells and expression analyzed using real-time RT-PCR.

Expression levels of target genes are determined in 4 different isolates of MPCs and 2 different isolates of hOBs. The MPCs (obtained from human bone marrow (Cambrex/Biowhittaker, Verviers, Belgium) and hOBs (obtained from Cambrex/Biowhittaker, Verviers, Belgium) are seeded at 3000 resp. 5000 cells/cm² in T180 flasks and cultured until they reached 80% confluency. The cells are washed with ice cold PBS and harvested by adding 1050 μl SV RNA Lysis Buffer to T180 flask. Total RNA is prepared using the SV Total RNA isolation System (Promega, Cat # Z3100). The concentration of the total RNA is measured with the Ribogreen RNA Quantification kit (Molecular Probes, Leiden, The Netherlands, Cat No. R-11490). cDNA synthesis is performed using 40 ng total RNA per reaction using the TaqMan Universal PCR Master Mix, No AmpErase UNG, kit (Applied Biosystems, Warrington, UK, Part number 4324018). For each reverse transcriptase (RT) reaction a minus-RT reaction (negative control: no enzyme included in the reaction) is performed.

The real-time reverse transcriptase (rtRT)-PCR reaction is performed with gene specific primers (Table 2) on both cDNA and minus-RT samples, using the SYBR Green PCR Master Mix (Applied Biosystems, Warrington, UK, Part number 4309155). Primers are quality controlled by performing PCR reactions on human genomic DNA and on plasmids containing the cDNA encoded by the gene studied. If the quality is unsatisfactory, additional primers are designed or validated primer sets are purchased (ABI). For the normalization of the expression levels a RT-PCR reaction is performed on human β-actin using the Human β-actin kit (Applied Biosystems, Warrington, UK, Part number 4310881E). The following program is run on a real-time PCR apparatus (ABI PRISM 7000 Sequence Detection System): 10 min at 25° C., 30 min at 48° C., 5 min at 95° C. Expression levels for the target genes in multiple MPC and hOB isolates are compared to expression levels of β-actin.

TABLE 2

Primers used for the expression
analysis of the target genes.

| | | | SEQ |
|---|---|---|---|
| Gene | Primer name | Sequence | ID |
| RORB | RORB_for#2 | TGCCGACTGCAGAAGTGTCTT | 33 |
| RORB | RORB_rev#2 | GTCCCTTTGCTTCTTGGACATC | 34 |
| NR1H3 | NR1H3_for#2 | GGGAAGACTTTGCCAAAGCA | 35 |
| NR1H3 | NR1H3_rev#2 | TCGGCATCATTGAGTTGCA | 36 |
| ADORA2A | ADORA2A_for | ATCCCGCTCCGGTACAATG | 39 |
| ADORA2A | ADORA2A_rev | TCCAACCTAGCATGGGAGTCA | 40 |
| RE2/GPR161 | RE2_for | ATTGCCATCGACCGCTACTATG | 43 |
| RE2/GPR161 | RE2_rev | CAGCCGATGAGCGAGTGAA | 44 |
| HSU93553 | HSU93553_for | CCGACAAGTGGTACATGGAAAG | 45 |
| HSU93553 | HSU93553_rev | CTCCGGCTTGTGATGCTATTATG | 46 |
| CD97 | CD97_for | ATCCAGGGTTCAGCTCTTTTTCT | 47 |
| CD97 | CD97_rev | TTTTCCGCATGACACTTTCG | 48 |
| GPR52 | GPRS2_for | TGCGTCCGAGCGTCACT | 49 |
| GPR52 | GPR52_rev | ATGCAGACATCCACCACACTGT | 50 |
| MC5R | MC5R_For | TCCGTGATGGACCCTCTCATATAT | 51 |
| MC5R | MC5R_rev | GGCAGCAAATAATCTCCTTAAAGGT | 52 |
| GPR65 | GPR65_for | CTTTGGTCACCATCCTGATCTG | 53 |
| GPR65 | GPR65_rev | TTCCTTGTTTTCCGTGGCTTTAT | 54 |
| GPR12 | GPR12_for | GCTGCCTCGGGATTATTTAGATG | 55 |
| GPR12 | GPR12_rev | TCTGGCTCTACGGCAGGAA | 56 |
| AVPR2 | AVPR2_for | TGTGAGGATGACGCTAGTGATTG | 57 |
| AVPR2 | AVPR2_rev | CAGCAACATGAGTAGCACAAAGG | 58 |
| DRD1 | DRD1_for | GTAACATCTGGGTGGCCTTTG | 59 |
| DRD1 | DRD1_rev | ACCTGTCCACGCTGATCACA | 60 |
| ESRRG | ESRRG_for | AAAGTGGGCATGCTGAAAGAA | 61 |
| ESRRG | ESRRG_rev | CGCATCTATCCTGCGCTTGT | 62 |
| OR51E2 | OR51E2_for | GCGGATCCCTCTTTTTTTTCC | 63 |
| OR51E2 | OR51E2_rev | GAGGACATTGGAGTGGCAGAA | 64 |

### EXAMPLE 4

### Analysis of the Up-Regulation of Endogenous Bone AP mRNA Versus that of Placental or Intestinal AP mRNA

Bone alkaline phosphatase (BAP) is the physiologically relevant alkaline phosphatase (AP) involved in bone formation. In order to determine whether the measured AP activities are due to up-regulation of BAP expression or of another AP, mRNA levels for all AP genes are analyzed after infection of MPCs.

mRNA levels are determined as described in the previous section. The difference is in the primer set used (Table 3): one set detects BAP ALPL (human alkaline phosphatase liver/bone/kidney) mRNA expression. Another set detects the expression of the 3 other AP genes (ALPI (human alkaline phosphatase intestinal), ALPP (human alkaline phosphatase placental (PLAP)), ALPPL2 (human alkaline phosphatase placental-like)). ALPI, ALPP and ALPPL2 are highly similar at the nucleotide level and can therefore be amplified using one primer pair.

TABLE 3

Primer sets used to analyze mRNA expression
of different alkaline phosphatase isoforms.

| | | SEQ |
|---|---|---|
| | | ID |
| Name | sequence | NO: |
| JDO-05F (PLAP) | TTCCAGACCATTGGCTTGAGT | 65 |
| JDO-05bis R | ACTCCCACTGACTTTCCTGCT | 66 |
| (PLAP/ALPI/ | | |
| ALPPL2) | | |
| JDO-21F (BAP) | CATGCTGAGTGACACAGACAAGAAG | 67 |
| JDO-21R (BAP) | TGGTAGTTGTTGTGAGCATAGTCCA | 68 |

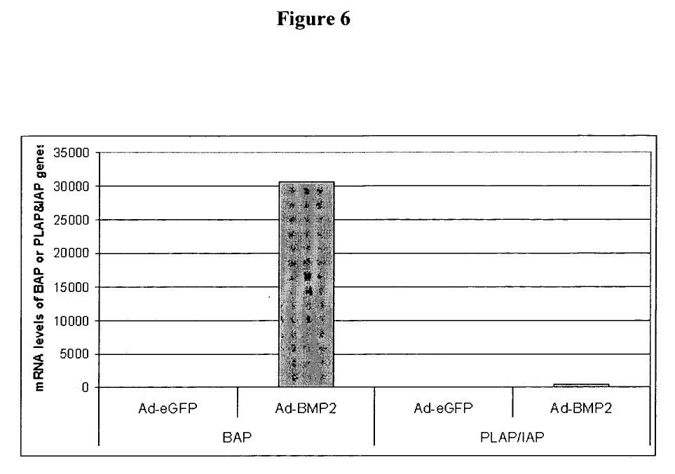

The primer pairs are first validated on RNA isolated from MPCs infected with Ad-eGFP and Ad-BMP2. FIG. 6 illustrates the strong up-regulation of BAP mRNA by Ad-BMP2 and the absence of up-regulation of expression of any of the other AP genes. Both primer sets are then used to measure mRNA levels for all AP genes in RNA isolated from Ad-target infected MPCs.

### EXAMPLE 5

### Analysis of Expression Levels of NR5A2, NR1H3, NR1H2, ESRRG in Cell Types Relevant to Bone Formation

To confirm that the identified target genes are endogenously expressed in cell types that relate to bone formation, mRNA levels for these genes are determined in relevant cell types.

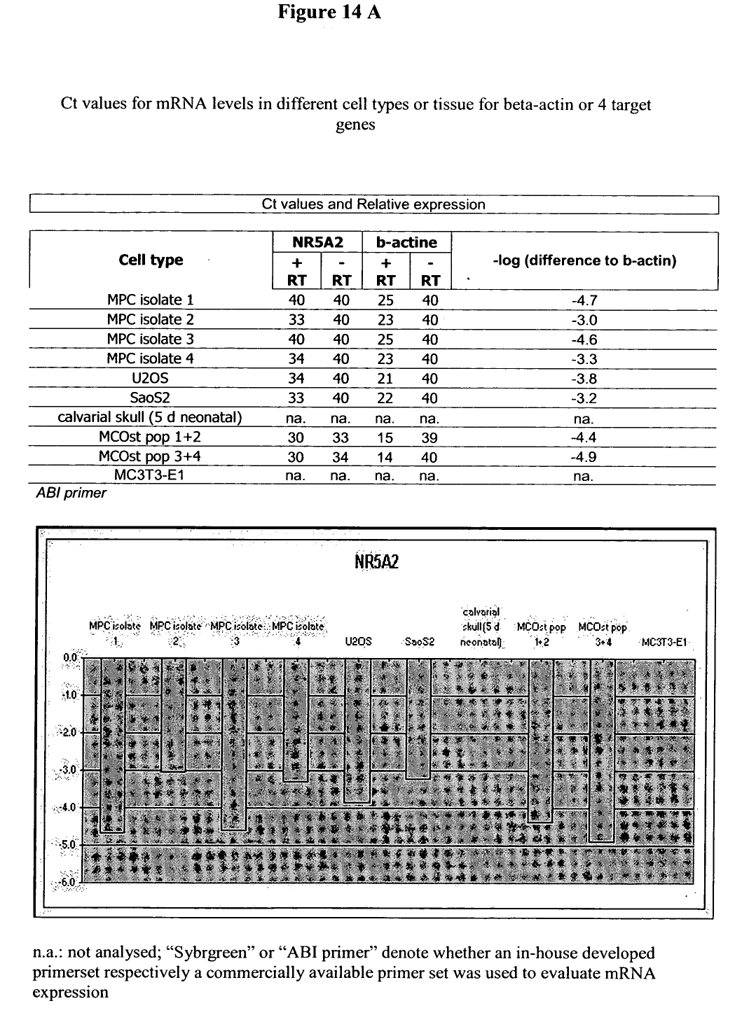

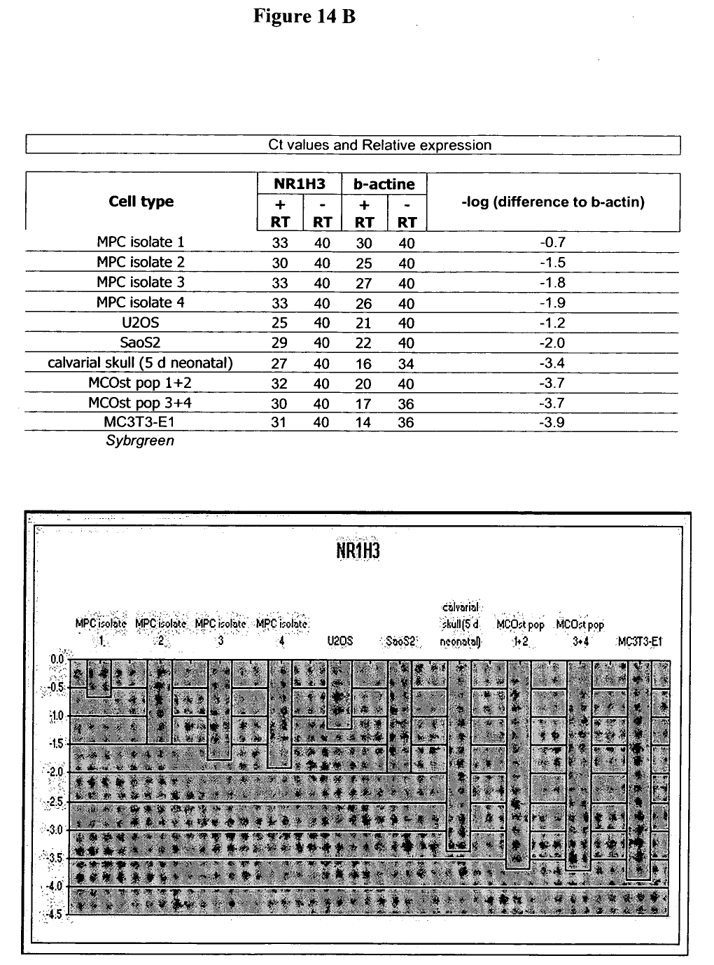

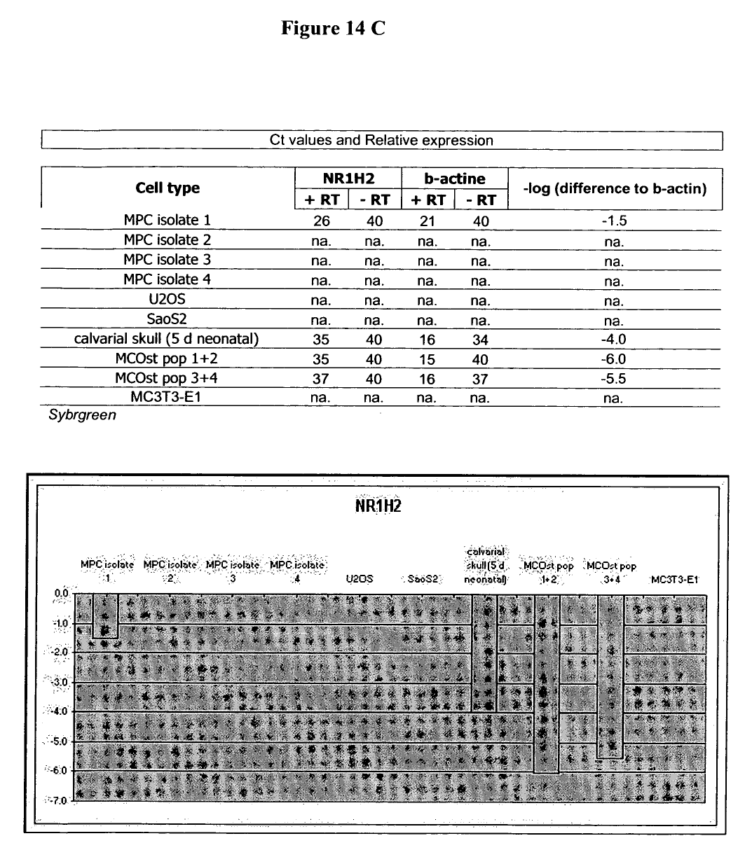

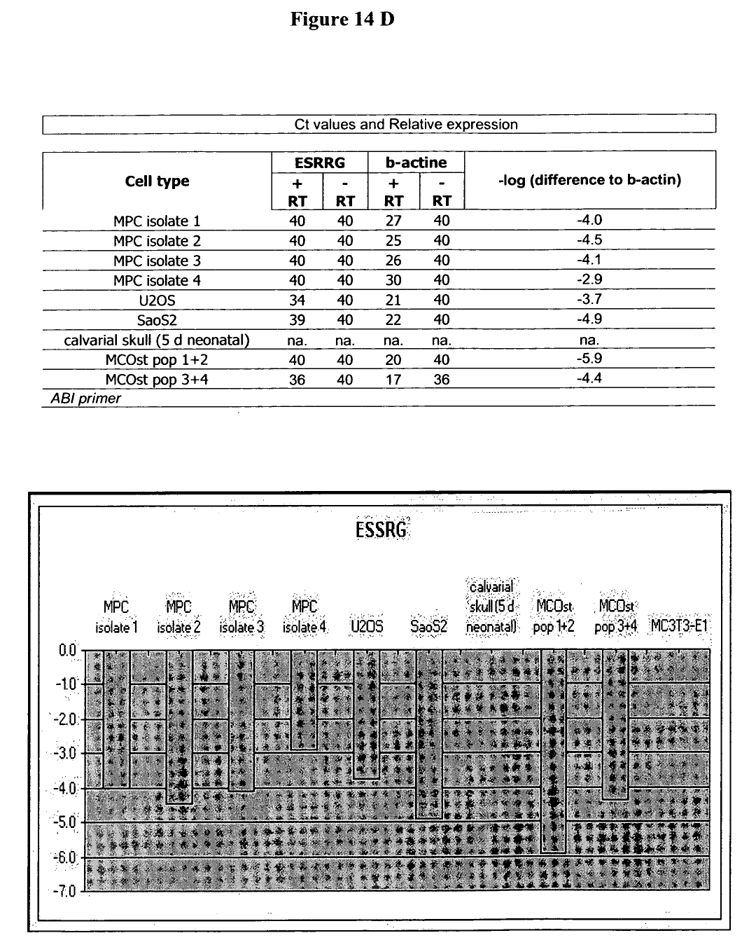

Primary cells or cell lines (FIG. 14A-D: MPC isolates 1-4, calvarial osteoblasts (MCOst pop 1+2, 3+4)), human osteoblast cell lines (SaOS2, U20S) are cultured or calvarial skull tissue is harvested from 5-day old mice. Monolayers or skull tissue is harvested and total RNA is extracted (SV Total RNA isolation System, Promega # Z3100) and quantified (Ribogreen RNA Quantification kit, Molecular Probes, Leiden). cDNA synthesis is performed using 20 ng total RNA per reaction using the TaqMan Universal PCR Master Mix, No AmpErase UNG, kit (Applied Biosystems, Warrington, UK, Part number 4324018). For each reverse transcriptase (RT) reaction a minus-RT reaction (negative control: no enzyme included in the reaction) is performed. The real-time reverse transcriptase (rtRT)-PCR reaction is performed with gene specific primers on both cDNA and minus-RT samples, using the SYBR Green PCR Master Mix (Applied Biosystems, Warrington, UK, Part number 4309155). Primers are quality controlled by performing PCR reactions on human genomic DNA and on plasmids containing the cDNA encoded by the gene studied if available. If the quality is unsatisfactory, additional primers are designed or validated, and primer sets are purchased (ABI). For the normalization of the expression levels a RT-PCR reaction is performed on human β-actin using the Human β-actin kit (Applied Biosystems, Warrington, UK, Part number 4310881E). The following program is run on a real-time PCR apparatus (ABI PRISM 7000 Sequence Detection System): 10 min at 25° C., 30 min at 48° C., 5 min at 95° C.

Expression levels for the four genes are compared to expression levels of beta-actin and the results shown in FIG. 14 A-D. The figures show the Ct values obtained for analysing mRNA levels in different cell types or tissue for beta-actin or 4 target genes; n.a.: not analysed; “Sybrgreen” or “ABI primer” denote whether an in-house developed primerset respectively a commercially available primerset was used to evaluate mRNA expression. Also shown are the graphic representation of the differential expression levels of target genes versus beta-actin expression levels (values are taken from left columns from the data tables).

In conclusion, the identified target genes are expressed in multiple cell types relevant to bone formation. It should be noted that target gene ESRRG is not expressed in the MPC isolates tested.

### EXAMPLE 6

### Activity of LXR Agonists in the BAP Assay, Upon Over-Expression of NR1H2 or NR1H3

Ad-NR1H2 and GW3965

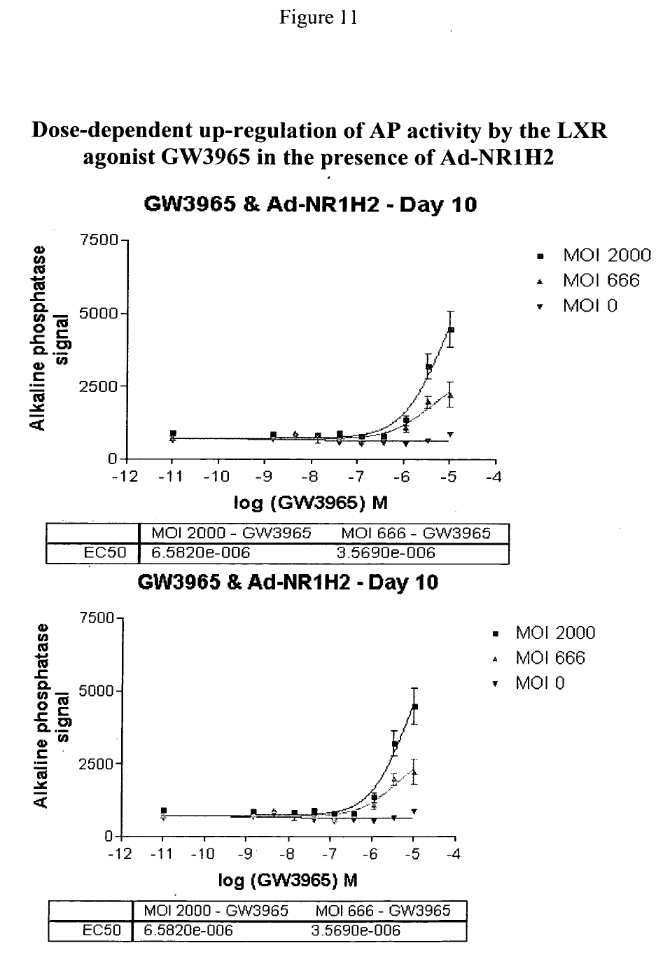

A dose-response relation is observed for AP activity when MPCs are infected with Ad-NR1H2 and treated with GW3965 (FIG. 11). MPCs are seeded on day 0 at 1000 cells per well of a 384 well plate and co-infected the next day using AdC51-hCAR (MOI 250) and different MOIs of Ad5-NR1H2 (MOI 2000, 666). On day 1, 9 concentrations (1.52E-9M, 4.57E-9M, 1.37E-8M, 4.12E-8M, 1.23E-7M, 3.7E-7M, 1.11E-6M, 3.33E-6M, 1E-5M) of the compound GW3965 with fixed vehicle concentration (DMSO at final concentration of 0.161%) are added to the wells. After 6 days, medium is removed and replaced with fresh medium containing the same concentrations of the compound GW3965. Readouts of AP activity are performed at several time points after the start of the experiment, typically after 7, 10 and 13 days. Up-regulation of alkaline phosphatase activity is read as follows: medium is removed from the monolayers, 15 μl MUP is added to each well, the plates are incubated for 15 min at 37° C. and then read for AP activity using a fluorescence plate reader (Fluostar, BMG). FIG. 11 illustrates the dose-response activity of GW3965 in the presence of Ad-NR1H2.

Ad-NR1H2, Ad-NR1H3 and Acetyl-Podocarpic Dimer (APD)

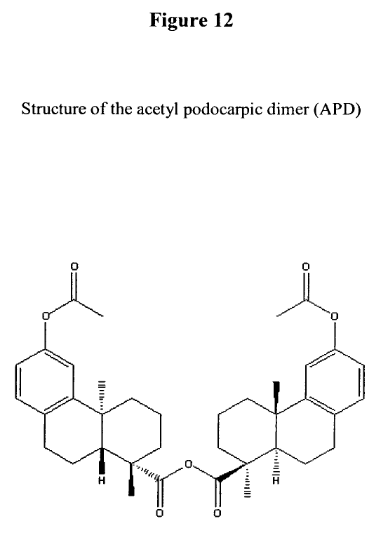

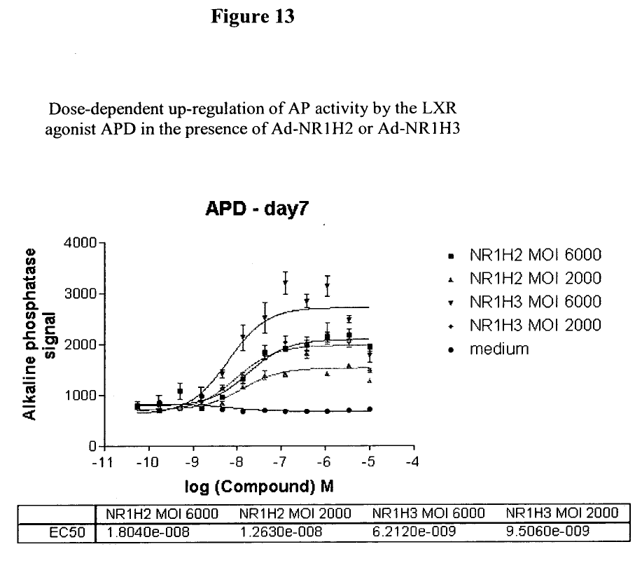

A dose-response relation is observed for AP activity when MPCs are infected with Ad-NR1H2 or Ad-NR1H3 and treated with acetyl podocarpic dimer (APD—see FIG. 12 for compound structure; APD is disclosed as “Compound 1” in published UA2003/0086923A1, of which the preparation of APD is incorporated by reference). MPCs are seeded on day 0 at 1000 cells per well of a 384 well plate and co-infected the next day using AdC51-hCAR (MOI 250) and different MOIs of Ad5-NR1H2 or Ad-NR1H3 (MOI 2000, 6000). On day 1, 12 concentrations (5.65E-11M, 1.69E-10M, 5.08E-10M, 1.52E-9M, 4.57E-9M, 1.37E-8M, 4.12E-8M, 1,23E-7M, 3.7E-7M, 1.11E-6M, 3.33E-6M, 1E-5M) of the compound APD with fixed vehicle concentration (DMSO at final concentration of 0.1%) are added to the wells. After 6 days, medium is removed and replaced with fresh medium containing the same concentrations of the compound APD. Readouts of AP activity are performed at several time points after the start of the experiment, typically after 7, 10 and 13 days. Up-regulation of alkaline phosphatase activity is read as follows: medium is removed from the monolayers, 15 μl MUP is added to each well, the plates are incubated for 15 min at 37° C. and then read for AP activity using a fluorescence plate reader (Fluostar, BMG). FIG. 13 illustrates the dose-response activity of APD in the presence of Ad-NR1H2 or Ad-NR1H3.

In conclusion, AP activity is up-regulated in cells transduced with either NR1H3 or NR1H2 in a dose-dependent manner when LXR agonists, APD, GW3965 and T0901317, respectively, are added to the cells at different concentrations in the AP assay.

### EXAMPLE 7

### Osteogenic Pathway Analysis: NR5A2 and NR1H3+T0901317 Up-Regulate mRNA Levels of Osteogenic Markers

Osteogenic differentiation of MPCs into osteoblasts is accompanied by the up-regulation of osteogenic proteins. The latter are useful to study the induction of osteogenic differentiation by a novel target using for example real-time RT-PCR. The MPCs that are used in this study are profiled for the up-regulation of a limited set of osteogenic markers by BMP2. Markers that show differential expression for BMP2 are subsequently tested against mRNA derived from Ad-NR5A2 infected cells or derived from Ad-NR1H3+T0901317 treated cells.

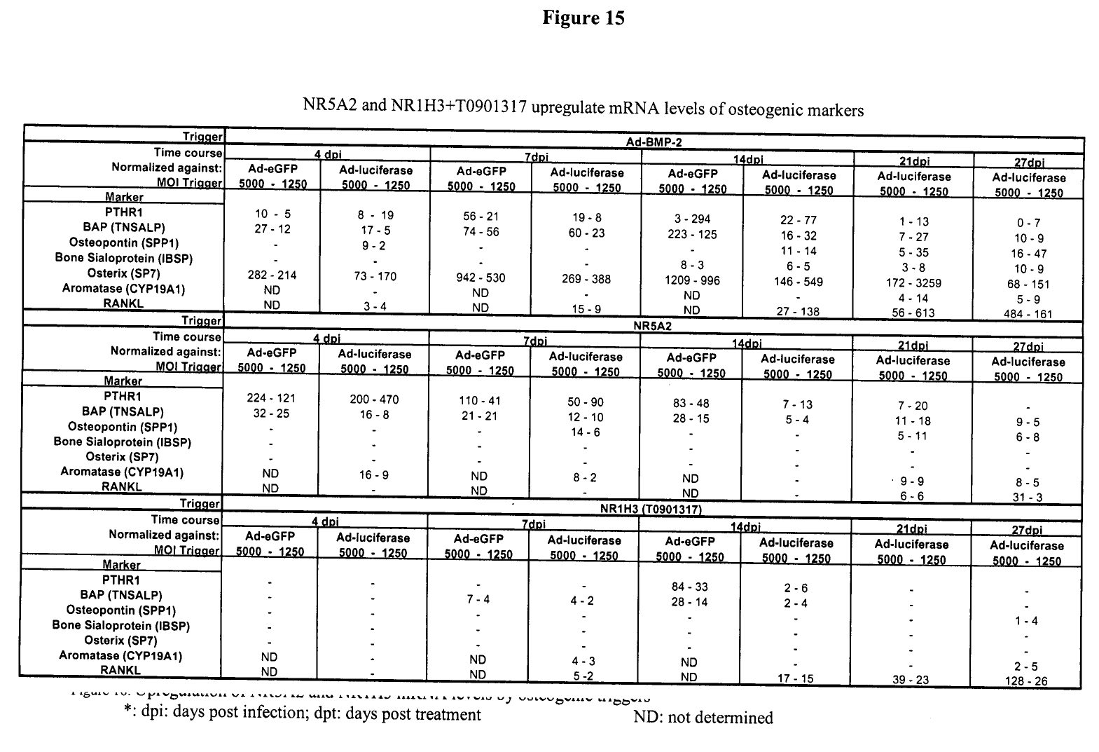

100,000 MPCs are seeded in each well of a 6 well plate in 2 ml MPC medium, containing 10% FCS. The next day, after incubation at 37° C., 10% CO₂ in a humidified incubator, cells are co-infected with AdC15-hCAR (final MOI of 750) and Ad-NR5A2, Ad-NR1H3+T0901317 (1 μM) or Ad-BMP2 (positive control) or Ad-eGFP or Ad-luciferase as negative controls (final MOIs of 1250 and 2500). Cells are incubated at 37° C., 10% CO₂ in a humidified incubator for a further six days unless cells are already harvested for RNA isolation. Virus is removed and replaced by 2 ml fresh OS medium (proprietary medium containing 10% FCS). Over the next 3 weeks, medium is refreshed 3 times per 2 weeks. Every other time, medium is refreshed half or completely. Monolayers are harvested at several time points (see FIG. 15), total RNA is harvested and quantified and rtRT-PCRs are run as follows: monolayers are washed with ice cold PBS and harvested by adding SV RNA Lysis Buffer. Total RNA is prepared using the SV Total RNA isolation System (Promega, Cat # Z3100). RNA concentration is measured with the Ribogreen RNA Quantification kit (Molecular Probes, Leiden, The Netherlands, Cat No. R-11490). cDNA synthesis is performed using 20 ng total RNA per reaction using the TaqMan Universal PCR Master Mix, No AmpErase UNG, kit (Applied Biosystems, Warrington, UK, Part number 4324018). For each reverse transcriptase (RT) reaction a minus-RT reaction (negative control: no enzyme included in the reaction) is performed. The real-time reverse transcriptase (rtRT)-PCR reaction is performed with gene specific primers on both cDNA and minus-RT samples, using the SYBR Green PCR Master Mix (Applied Biosystems, Warrington, UK, Part number 4309155). Primers are quality controlled by performing PCR reactions on human genomic DNA and on plasmids containing the cDNA encoded by the gene studied if available. If the quality is unsatisfactory, additional primers are designed or validated primer sets are purchased (ABI). For the normalization of the expression levels a RT-PCR reaction is performed on human P-actin using the Human P-actin kit (Applied Biosystems, Warrington, UK, Part number 4310881E). The following program is run on a real-time PCR apparatus (ABI PRISM 7000 Sequence Detection System): 10 min at 25° C., 30 min at 48° C., 5 min at 95° C.

Expression levels for osteogenic marker genes are first normalized for beta-actin levels. The resulting data for Ad-BMP2, Ad-NR5A2 and Ad-NR1H3+T0901317 (1 μM) samples are then compared to those of Ad-eGFP or Ad-luciferase negative control samples, harvested at the same time points, for cells infected at the same MOI. The fold up-regulation of marker gene mRNA induced by NR5A2 or BMP2 over-expression are calculated and presented in FIG. 15. Osteogenic markers are considered to be up-regulated by BMP2, NR5A2 or NR1H3+T0901317 over-expression if their expression is 4-fold higher than that in a negative control sample (Ad-eGFP or Ad-luciferase). Ad-NR5A2 up-regulated expression of PTHR1, BAP, osteopontin, aromatase and RANKL at one or more time points studied. Ad-NR1H3+T0901317 up-regulated expression of PTHR1, BAP, osteopontin, aromatase and RANKL at one or more time points studied.

### EXAMPLE 8

### Osteogenic Pathway Analysis: Up-Regulation of NR5A2 and NR1H3 mRNA Levels by Osteogenic Triggers

MPCs are treated with established inducers of osteogenesis and NR5A2 or NR1H3 mRNA levels are determined in an effort to place NR5A2 or NR1H3 in known osteogenic pathways.

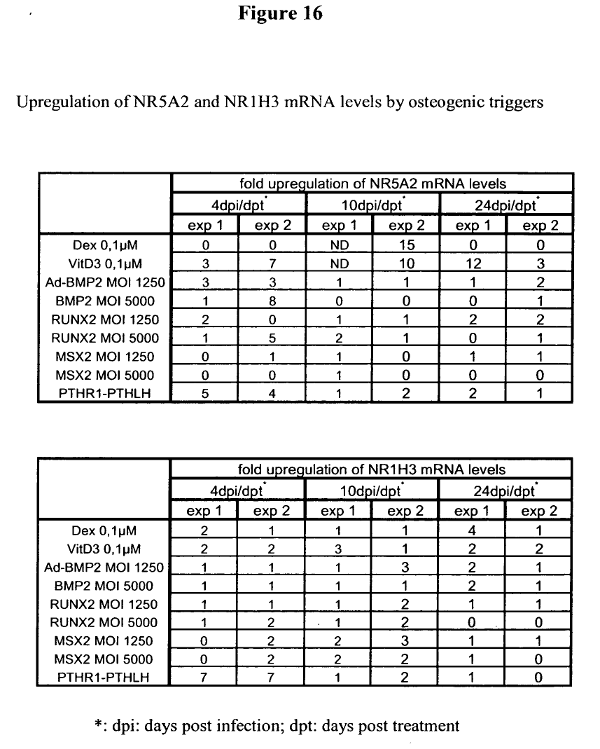

100,000 MPCs are seeded in each well of a 6 well plate in 2 ml MPC medium, containing 10% FCS. The next day, after incubation at 37° C., 10% CO₂ in a humidified incubator, cells are co-infected with AdC15-hCAR (final MOI of 750) and Ad-BMP2, Ad-RUNX2, Ad-MSX2, Ad-PTHR1/PTHLH or Ad-eGFP or Ad-luciferase as negative controls (final MOIs of 1250 and 2500). Alternatively, cells are treated with dexamethasone (final concentration 0.1 μM), VitD3 (final concentration 0.1 μM) or the vehicle controls (0.1% EtOH or DMSO). Cells are incubated at 37° C., 10% CO₂ in a humidified incubator for a further six days unless cells are already harvested for RNA isolation. Virus is removed and replaced by 2 ml fresh OS medium (proprietary medium containing 10% FCS). Over the next 18 days, medium is refreshed 3 times per 2 weeks. Every other time, medium is refreshed half or completely. Monolayers are harvested at several time points (see FIG. 16), total RNA is harvested and quantified and rtRT-PCRs is run as described in the previous example “NR5A2 and NR1H3+T0901317 up-regulate mRNA levels of osteogenic markers”. The fold up-regulation of NR5A2 or NR1H3 mRNA compared to negative controls (vehicle for dexamethasone or VitD3 treatment) or Ad-luciferase for Ad-infections) is calculated (FIG. 16).

NR5A2 mRNA levels became up-regulated by VitD3 treatment at several time points and NR1H3 and NR5A2 levels by Ad-PTHR1/PTHLH infection at the 4 dpi time point.

### EXAMPLE 9

### Mineralization Assay

The process of osteogenesis consists of several successive events. During the initial phases of osteogenesis, BAP becomes up-regulated, while mineralization is a specific event occurring in later stages of osteogenesis.

Bone tissue consists of cells embedded in a matrix of organic materials (e.g., collagen) and inorganic materials (Ca²⁺ and phosphate). Bone mineralization is shown in vitro by staining differentiated bone cells for the matrix they deposited. The Von Kossa and Alizarin RedS stains allow the visualization of deposited phosphate and calcium, respectively.

On day one, primary human MPCs are seeded in a 6 well plate (Costar or Nunc) at a density of 50,000 to 250,000 cells per well, typically at 100,000 cells per well. MPCs are co-infected one day later with AdC15-hCAR (MOI 750) and Ad-control (eGFP or BMP2) or hit-virus (Ad5) (at MOIs between 250 and 20,000, typically at MOIs 5000 and 2500). For Ad-GPCR or Ad-NHR experiments, cells can additionally be treated with specific ligands. These are added at the EC₅₀ concentration and at concentrations 5-10 times higher and lower. Ligands are added 2-3 times per week. Medium supplemented with 100 μg/ml L-ascorbate and 10 mM beta-glycerophosphate, is refreshed 2 times a week. 20 to 30 days after the start of the experiment, cells are stained with Von Kossa stain or with Alizarin RedS stain.

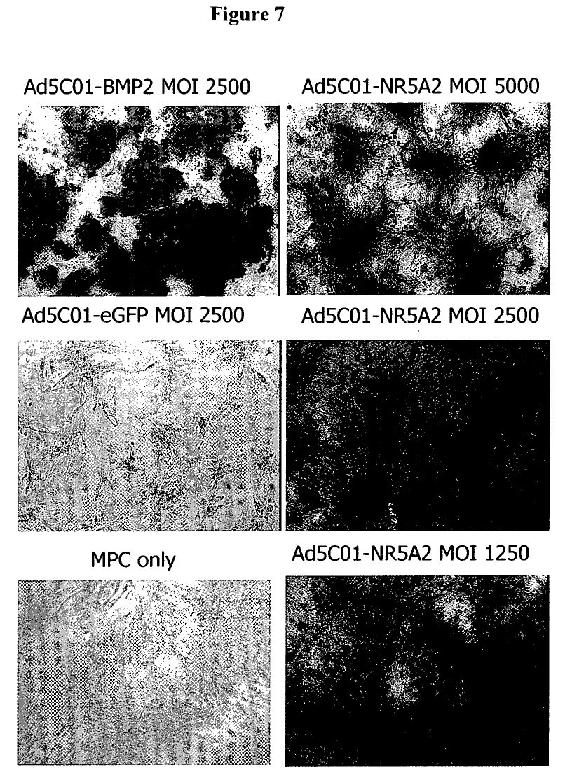

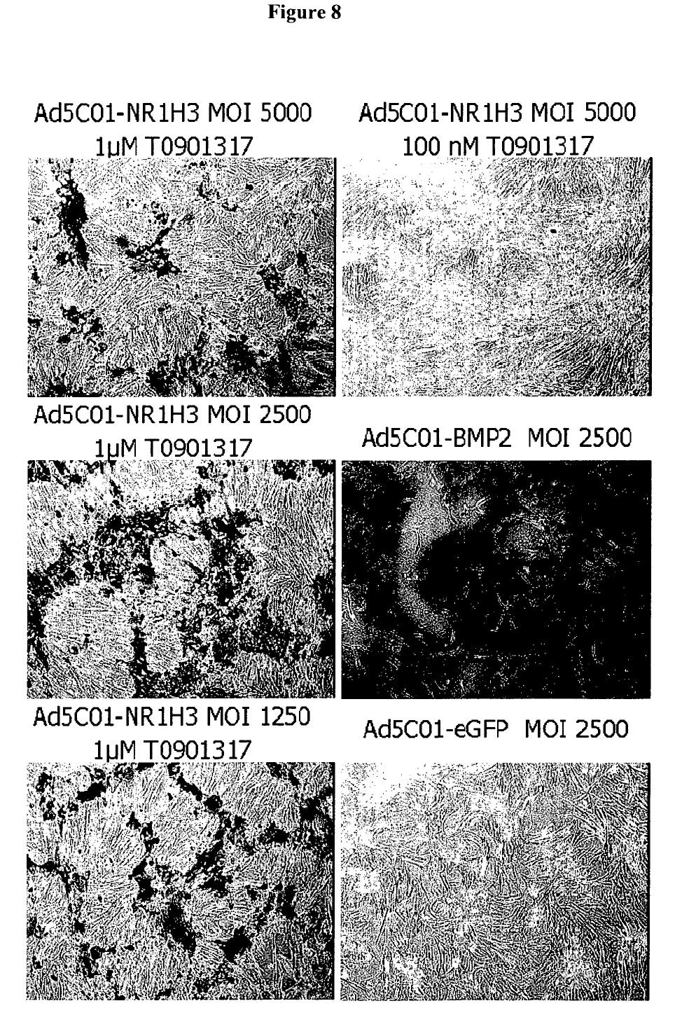

The Alizarin RedS staining is carried out as follows: cells are washed once with PBS, fixed with 10% paraformaldehyde for 45 minutes at 4° C., and washed 2 times with PBS. Cells are incubated with 40 mM aqueous Alizarin RedS solution, pH 4.1-4.3 for 10 minutes followed by 5 washes with distilled water. Staining is evaluated and photographed using white light. Examples are shown in FIGS. 7 and 8.

In conclusion, two targets are already identified that induced mineralization, in the presence or absence of their respective ligands: NR5A2 (FIG. 7) and NR1H3 (FIG. 8).

In studies conducted with calvarial skull tissue, the administering of LXR agonists alone induce bone formation, thereby showing that LXR agonists are useful in the methods of the present invention, including methods for differentiating precursor cells into osteoblasts, for stimulating bone tissue formation, and treating or preventing bone diseases, including treating or preventing osteoporosis.

The data presented in FIGS. 9 and 10 indicate that LXR agonists do not induce the same level of alkaline phosphatase activity in the absence of Ad-NR1H3 or Ad-NR1H2, as in the presence of Ad-NR1H3 or Ad-NR1H2. This finding, which appears inconsistent with the calvarial skull tissue findings, may be the result of many factors, such as, for example, the overexpression of NR1H3 or NR1H2 protein may recruit a different set of coactivator proteins than endogenous NR1H3 or NR1H2 proteins.

### EXAMPLE 10

### Calvarial Skull Assay: Activity of the NR1H3 Agonist T0901317

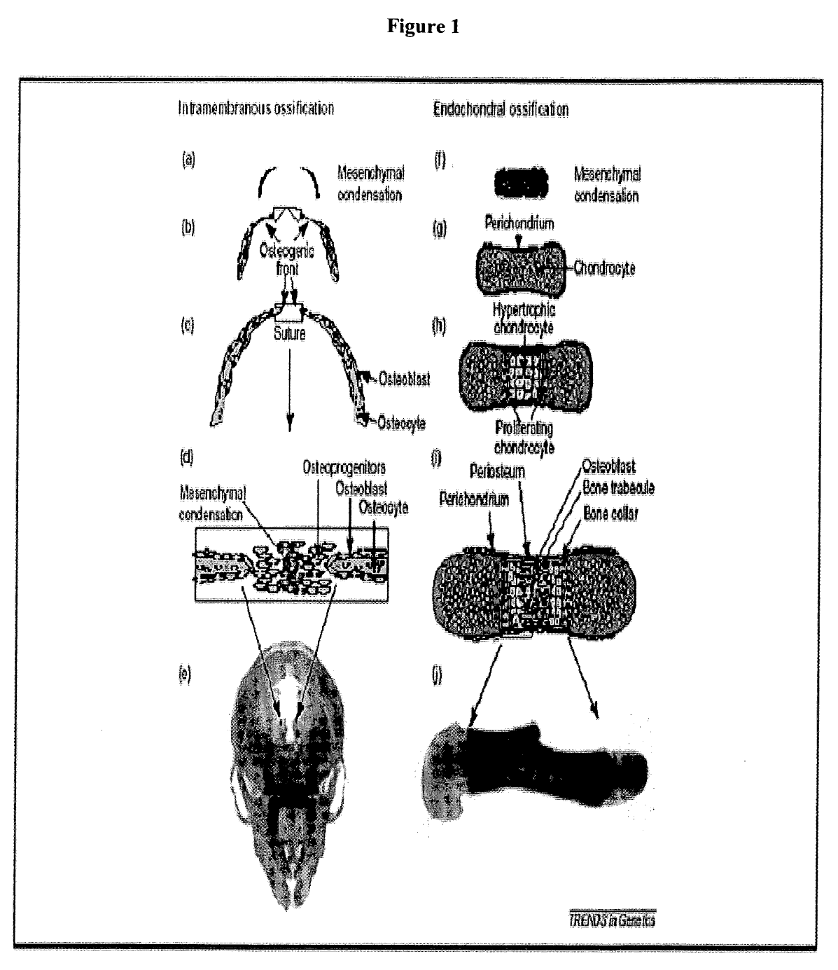

Adult bone consists of organic (e.g. collagen type I) and inorganic (calcium phosphate) material, bone-forming cell types (MPCs, osteoblasts and osteocytes) and bone-degrading cell types (osteoclasts). Since the MPC monolayers, used in the identification and initial validation of the target hits, do not mimic the multi-cellular 3-dimensional in vivo environment, bone organ culture models were developed. Elegant ex vivo models that closely mimic the in vivo bone environment are bone organ cultures, such as the metatarsal or calvarial skull organ culture models. In the former model, foot bones formed by endochondral ossification are used. In the latter model, skull bones, formed by intramembranous ossification are used (see also FIG. 1). This example describes the latter model using calvarial skull bones.

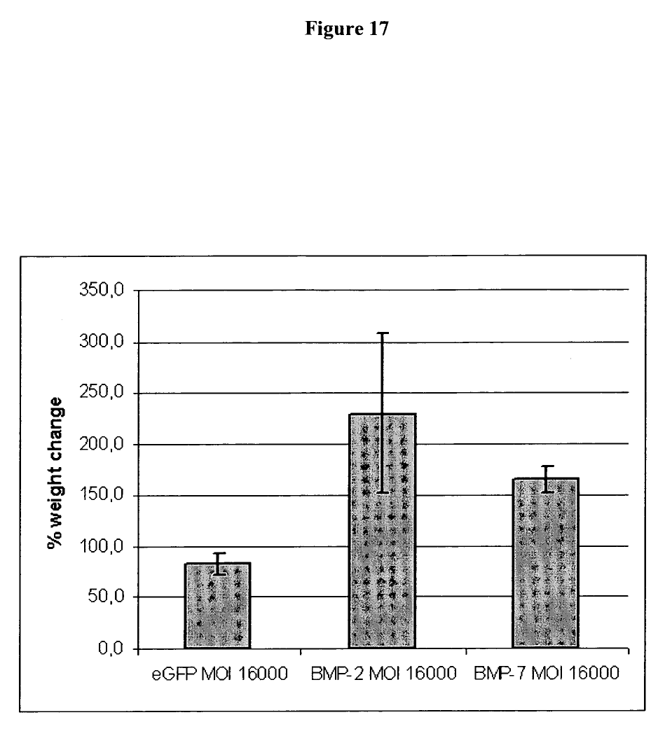

CD1 pups are harvested around birth from CD1 female mice (received from Janvier (Le Genest St Isle, France) when they were 11 days pregnant). Pups are decapitated and the calvarial skull is dissected and split into 2 hemicalvaria. Hemicalvaria are blotted using sterile gauze, weighed and cultured in 24 well plates (MEMalpha or BGJb-Fitton-Jackson medium containing 50 μg/ml L-ascorbic acid (Sigma, A-4034), 5 mM β-glycerophosphate (Sigma, G-9891) and Penicillin-Streptomycin (Invitrogen Cat # 15140-122)). Small molecules (ligands, agonists, antagonists) are tested in at least three-fold at a minimum of 3 concentrations. Each small molecule is added to the medium on day 0 and added again when refreshing the medium (every 2-3 days). Three to 16 days after the start of the experiment, skulls are weighed again after blotting them dry using sterile gauze. The weight difference is calculated, expressed as percent weight change and the mean and standard deviations (SD) are calculated for the triplicate measurements. Data are analyzed using the Student's t-test. Weight increases for Ad-BMP2 and Ad-BMP7 positive controls are depicted in FIG. 17.

The formation of new osteoid is analyzed histologically as follows: hemicalvaria are fixed in 10% buffered formalin for at least 2 days, decalcified in 10% EDTA overnight, processed through graded alcohols and embedded in paraffin wax. Three to 10 μm sections are prepared of the calvaria and stained with hematoxylin and eosin (H&E). Healthy cells, dead cells, old and new bone, and collagen are identified by their distinctive morphology and colouring observed after H&E staining. The surfaces taken by these are measured stereologically (μm² readout) and termed Osteoblast area, Debris area, Native and New bone area, Collagen area and Total area (sum of the previous 5 areas), respectively. In addition, the thickness (μm readout) is measured at 8 positions, evenly spaced over the section.

The histological readout of the calvarial skull assay is developed using known osteogenic agents. Hemicalvaria were treated with recombinant human parathyroid hormone (rhPTH). PTH has a dual action on bone: PTH needs to be administered in vivo intermittently rather than continuously since the latter treatment regimen results in bone resorption, while the former results in bone build-up. This dual action is also observed in the calvarial skull model as expected: PTH at 10⁻⁷ M has a resorptive effect on bone tissue but induces bone build-up at 10⁻¹¹ M.

Since NR1H3 and T0901317 score well in the AP and mineralization assay, the commercially available NR1H3 agonist, T0901317, is tested in the calvarial skull model to further show the osteogenic potential of NR1H3 agonism.

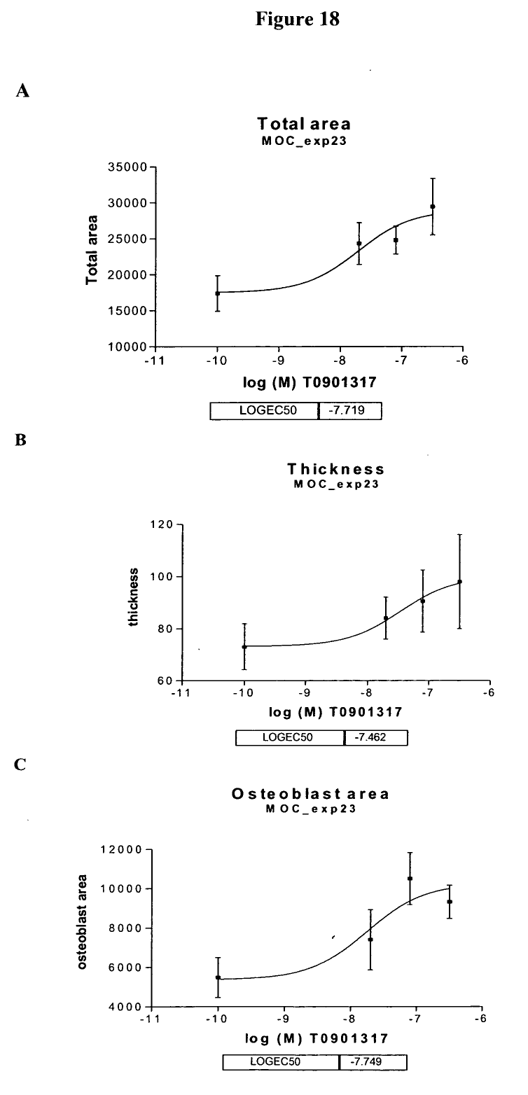

T0901317 is added to the culture medium of the dissected hemicalvaria at the day of dissection at several doses (19.5, 78.1 and 313 nM), in fourfold. The concentration of the solvent (vehicle), DMSO, is fixed at a final concentration of 0.05%. The medium, containing T0901317 or vehicle control is refreshed every 2-3 days. Hemicalvaria are harvested 7 days after the initiation of the experiment and subjected to the histological analysis described above. Statistically significant increases are observed for areas of osteoblast, collagen and new bone. Dose-response activity of the compound is observed towards areas of osteoblast, total area (sum of all areas measured) and thickness (FIG. 18).

Apart from the H&E stainings, other stainings are routinely done. In one method, AP activity is visualized as follows: slides are fixed for 10 min using 4% paraformaldehyde and washed with PBS and MilliQ water. Slides are incubated for 5 min with ALP buffer (ALP buffer: 0.1M Tris-HCl pH 9.5, 20 mM MgCl₂, 100 mM NaCl), blotted using tissue and incubated with substrate (NBT/BCIP (Nitrobluetetrazolium chloride/5-bromo-4-chloro-3-indolyl phosphate, Roche) in ALP buffer). The staining is stopped by washing with MilliQ water when the color turns from yellow into brown.

### EXAMPLE 11

### Dominant-Negative RUNX2 Mutant Interferes With AP Up-Regulation by NR5A2, NR1H3+T0901317 and ESRRG

RUNX2 is a key osteogenic transcription factor relaying many osteogenic triggers received by MPCs or osteoblasts into the appropriate osteogenic transcriptional output. Knockout studies in mice show that RUNX2 is crucial for the ossification of the skeleton during development (Franceschi RT and Xiao G (2003)).

A useful tool to study RUNX2 biology and the osteogenic signals it relays are RUNX2 mutants. A truncated RUNX2 protein lacking the C-terminal transactivating region, but retaining the N-terminal Runt homology DNA binding domain acts as a dominant-negative RUNX2 (DN-RUNX2) protein. This type of mutant can interfere with RUNX2 activity in vitro and in vivo (Zhang et al., 2000). MPCs express significant levels of RUNX2 mRNA (levels are about 10-fold lower than b-actin mRNA levels).

Since the osteogenic activity of BMP2 is known to work through RUNX2, Ad-BMP2 and Ad-DN-RUNX2 viruses are used to develop the DN-RUNX2 assay. The human full-length RUNX2 cDNA is obtained by RT-PCR from total RNA extracted from MPCs. The 5′ part of the cDNA encoding amino acids 1-214 is obtained by PCR from the cloned RUNX2 cDNA and subcloned in an adenoviral adapter plasmid. The identity of the cloned fragment is verified by sequencing. This plasmid is used to generate an adenoviral stock, as described in WO 9964582.

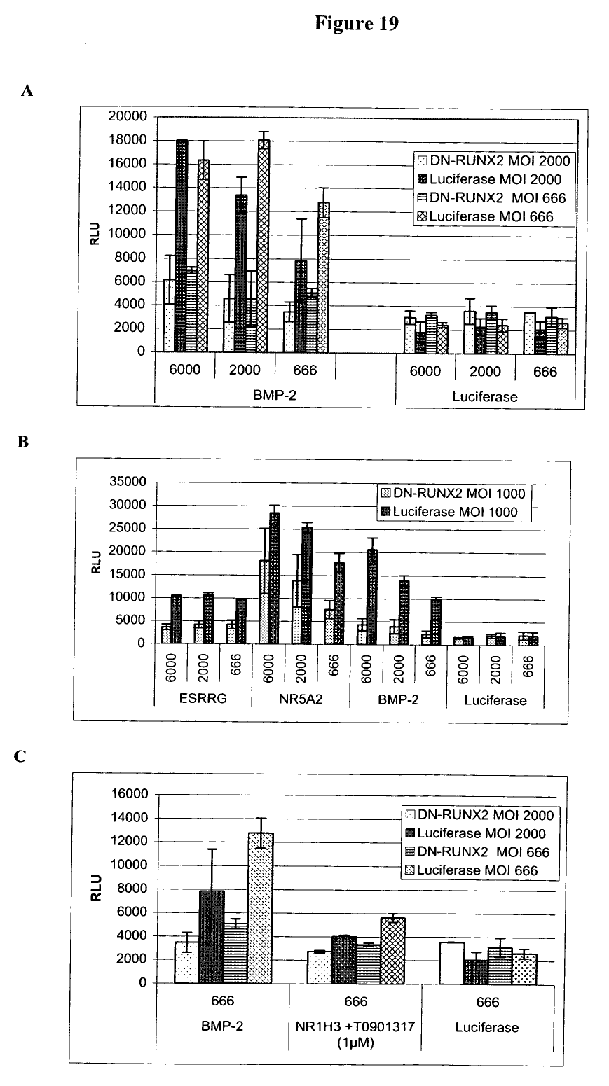

MPCs are seeded at 1000 cells/well in a 384 well plate and infected the next day with adenoviruses encoding hCAR (MOI 250), Ad-BMP2 (MOIs 6000, 2000, 666) and one of Ad-DN-RUNX2 or Ad-luciferase (MOIs 2000 or 666). Alkaline phosphatase activity is read 6 days post infection. From FIG. 19 (A), it is clear that overexpression of DN-RUNX2 significantly reduces the BMP2-induced up-regulation of AP activity. This result shows the functionality of the DN-RUNX2 construct used.

The DN-RUNX2 assay is used to test the involvement of RUNX2 in the up-regulation of AP activity by NR5A2, NR1H3, and ESRRG. MPCs are seeded at 1000 cells/well in a 384 well plate and are infected the next day with adenoviruses encoding hCAR (MOI 250), Ad-BMP2, Ad-ESRRG, Ad-NR5A2, Ad-NR1H3 (MOIs 6000, 2000, 666) and one of Ad-DN-RUNX2 or Ad-luciferase (MOI 1000 or MOIs 2000 and 666) (see FIG. 19(C)). Alkaline phosphatase activity is read 6 days post infection and raw data are analysed. From FIG. 19(B), it is clear that overexpression of DN-RUNX2 significantly reduced the ESRRG- and NR5A2-induced up-regulation of AP activity. From FIG. 19(C), it is clear that overexpression of DN-RUNX2 significantly reduces the up-regulation of AP activity induced by NR1H3 in the presence of T0901317.

### EXAMPLE 12

### Induction of Alkaline Phosphatase Activity by NR5A2, NR1H3+T0901317, ESRRG, Independent of MPC Isolate

MPCs can be isolated, with informed consent, from fresh bone marrow isolated from healthy donors (Cambrex Bioscience/Biowhittaker, Verviers, Belgium). MPCs are a physiologically relevant cell type to isolate osteogenic factors in vitro, using e.g. the AP assay (see Example 2). To exclude targets that function in only one MPC isolate (i.e. from one donor), the targets are also tested on several different MPC isolates to exclude the influence of genetic background in the target discovery process using MPCs.

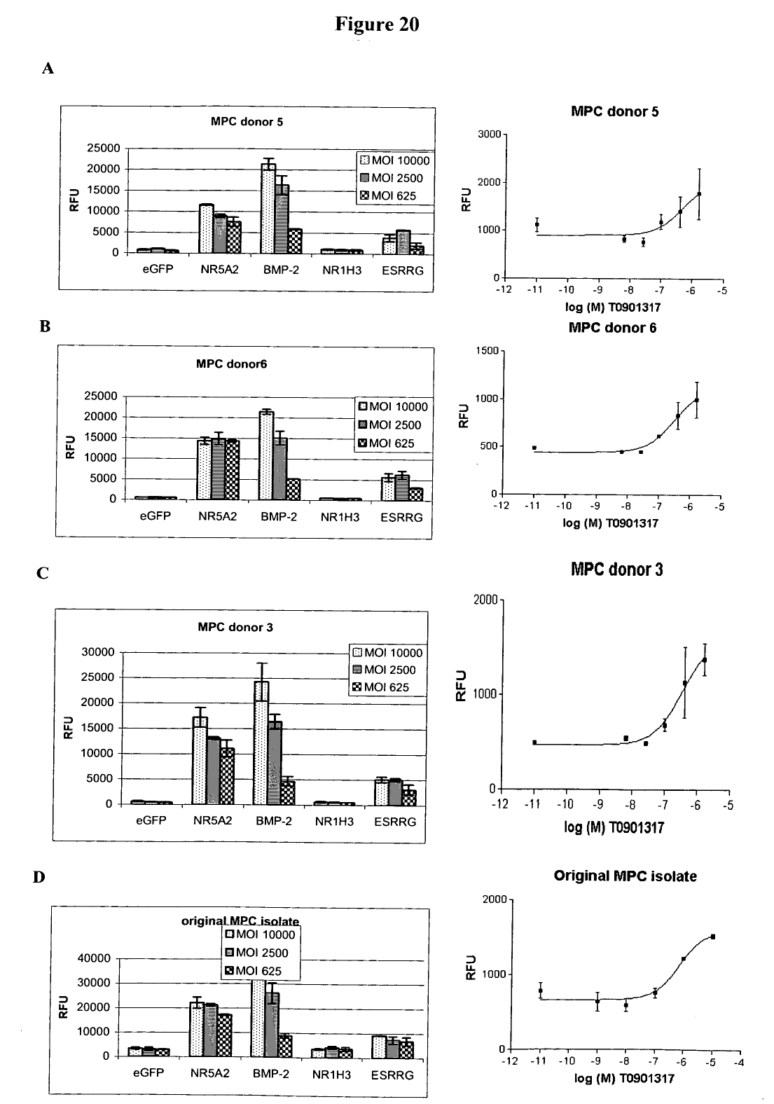

The osteogenic factors NR5A2, NR1H3 and ESRRG are tested in 3 independent MPC isolates different from the one used for target discovery in the AP assay according to a protocol described in Example 2. MPCs are seeded at 1000 cells/well of a 384 well plate and infected the next day with adenoviruses encoding hCAR (MOI 250), Ad-BMP2, Ad-ESRRG, Ad-NR5A2, and Ad-NR1H3 (MOIs 10000, 2500, 625). MPCs infected with Ad-NR1H3 virus at MOI 2500 are also treated one day after infection with T0901317 at different concentrations (FIG. 20) or vehicle. MPCs isolated from 4 different donors (A,B,C,D), are infected with Ad-hCAR, Ad-BMP2 (positive control), Ad-eGFP (negative control), Ad-NR5A2, Ad-ESRRG (data presented in the left panels of A,B,C,D) and Ad-NR1H3+T0901317 (data presented in the right panels of A,B,C,D) together with Ad-luciferase or Ad-DN-RUNX2. 6 days after the start of the infection, endogenous AP activity is measured.

From FIG. 20, it is clear that NR5A2, NR1H3+T0901317 and ESRRG induce AP activity to similar extents in all 4 MPC isolates tested.

### EXAMPLE 13

### Analysis of LXR Agonists for the Treatment of Osteoporosis in the Ovariectomy Animal Model

The gold-standard animal model for analysis of potential osteoporosis therapeutics is the ovariectomy model. Ovariectomy (OVX) results in a drop in estrogen production which is an important causative factor of osteoporosis. This example uses the rat as the animal model, but other animal models such as mice or primates are routinely used by those skilled in the art.

Three-month-old female Lewis rats are maintained under constant conditions of temperature (20±1° C.) and light (12-h light-dark cycle) with ad libitum access to food and water. Rats are sham operated or underwent bilateral ovariectomy after being anesthetized with ketamine and Xylazine. Ovaries are removed after ligation of the uterine horn.

The following groups are formed: sham operated control rats (N=10), ovariectomized rats that receive saline only (OVX, N=12), ovariectomized rats that receive 17β-estradiol (Sigma Chemical Co., St. Louis, Mo., USA) dissolved in small amounts of ethanol with the volume adjusted with olive oil to give a concentration of 30 μg/kg body weight and administered daily subcutaneously for 6 weeks (OVX-E, N=11), ovariectomized rats that receive LXR agonists suspended in the appropriate vehicle (e.g. water and lecithin) and administered daily p.o. for 6 weeks at a dose of 0.1 to 100 mg/kg body weight (OVX-A, N=8). All rats are sacrificed after 6 weeks. On the 2nd, 3^{rd} and 28th day prior to sacrifice, the rats receive xytetracycline (Terramycin, Pfizer) administered intramuscularly at a dose of 20 mg/kg for bone labeling. Femora are obtained for mineralized bone histology and histomorphometry. Bone mineral density (BMD) is measured by dual-energy X-ray absorptiometry (using e.g. apparatus from CTI Concord Microsystems, Knoxville Tenn. http://www.ctimi.com) adapted to the measurement of BMD in small animals. A distal femur scan is performed. In vivo reproducibility is evaluated by measuring the coefficient of variation (CV=100×SD/mean) of five BMD measurements in one rat weighing about 220 g, each time repositioning the rat at the two different sites. The variation is 1.4% in distal femur. In addition, bone alveolar structure is evaluated. All parameters are measured twice, i.e., at baseline and after 6 weeks.

The distal right femur is fixed in 70% ethanol, dehydrated, embedded in methylmethacrylate, and sectioned longitudinally using a Policut S microtome (Reichert-Jung, Heidelberg, Germany). 5- and 10-μm sections are obtained from the center of each specimen. The 5-μm section is stained with 0.1% toluidine blue, pH 6.4, and at least two non-consecutive sections are examined for each sample. Static and structural parameters of bone formation and resorption are measured at a standardized site below the growth plate in the secondary spongiosa.

Urine is collected in metabolic cages. Urinary deoxypyridinoline is measured by ELISA and creatinine via a third party diagnostic laboratory. Other plasma markers are evaluated by ELISA included osteocalcin, bone sialoprotein, BMP (bone morphometric protein) and the catabolic marker carboxy-terminal-telopeptide.

The rats are sacrificed by exsanguination while under ether anesthesia. All animal data is obtained by blind measurements. Data are reported as mean±standard deviation (SD). The paired Student t-test is used to analyze values within the same group at baseline and after 6 weeks. ANOVA followed by the Newman-Keuls post-test is used to compare different groups. Linear regression between histomorphometric variables and non-invasive bone mass measurements is calculated and the Pearson test is applied. Statistical significance is set at P values lower than 0.05.

### EXAMPLE 13

### Analysis of Targets Agonists for the Treatment of Osteoporosis in the Ovariectomy Animal Model

The gold-standard animal model for analysis of potential osteoporosis therapeutics is the ovariectomy model. Ovariectomy (OVX) results in a drop in estrogen production which is an important causative factor of osteoporosis. The example below relates to the rat as the animal model, but other animal models such as mice or primates are routinely used as well by those skilled in the art.

Three-month-old female Lewis rats are maintained under constant conditions of temperature (20±1° C.) and light (12-h light-dark cycle) with ad libitum access to food and water. Rats are sham operated or underwent bilateral ovariectomy after being anesthetized with ketamine and Xylazine. Ovaries are removed after ligation of the uterine horn.

The following groups are formed: sham operated control rats (N=10), ovariectomized rats receiving saline only (OVX, N=12), ovariectomized rats receiving 17β-estradiol (Sigma Chemical Co., St. Louis, Mo., USA) dissolved in small amounts of ethanol with the volume adjusted with olive oil to give a concentration of 30 μg/kg body weight and administered daily subcutaneously for 6 weeks (OVX-E, N=11), ovariectomized rats receiving agonists of the targets of this application suspended in the appropriate vehicle (e.g. water and lecithin) and administered daily p.o. for 6 weeks at a dose of 0.1 to 100 mg/kg body weight (OVX-A, N=8). All rats are sacrificed after 6 weeks. On the 2nd, 3^{rd} and 28th day prior to sacrifice, they receive xytetracycline (Terramycin, Pfizer) administered intramuscularly at a dose of 20 mg/kg for bone labeling. Femora are then obtained for mineralized bone histology and histomorphometry. Bone mineral density (BMD) is measured by dual-energy X-ray absorptiometry (using e.g. apparatus from CTI Concord Microsystems, Knoxville Tenn.http://www.ctimi.coin/) adapted to the measurement of BMD in small animals. A distal femur scan is performed. In vivo reproducibility is evaluated by measuring the coefficient of variation (CV=100×SD/mean) of five BMD measurements in one rat weighing 220 g, each time repositioning the rat at the two different sites. The variation is 1.4% in distal femur. In addition, bone alveolar structure is evaluated. All parameters are measured twice, i.e., at baseline and after 6 weeks.

The distal right femur is fixed in 70% ethanol, dehydrated, embedded in methylmethacrylate, and sectioned longitudinally using a Policut S microtome (Reichert-Jung, Heidelberg, Germany). 5- and 10-μm sections are obtained from the center of each specimen. The 5-μm section is stained with 0.1% toluidine blue, pH 6.4, and at least two non-consecutive sections are examined for each sample. Static and structural parameters of bone formation and resorption were measured at a standardized site below the growth plate in the secondary spongiosa.

Urine is collected in metabolic cages. Urinary deoxypyridinoline is measured by ELISA and creatinine via a third party diagnostic laboratory. Other plasma markers are evaluated by ELISA included Osteocalcin, Bone sialoprotein, BMP (bone morphometric protein) and the catabolic marker Carboxy-Terminal-Telopeptide.

The rats are then sacrificed by exsanguinations while under ether anesthesia. All animal data is obtained by blind measurements. Data are reported as mean±standard deviation (SD). The paired Student t-test is used to analyze values within the same group at baseline and after 6 weeks. ANOVA followed by the Newman-Keuls post-test is used to compare different groups. Linear regression between histomorphometric variables and non-invasive bone mass measurements is calculated and the Pearson test is applied. Statistical significance is set at P values lower than 0.05.

### EXAMPLE 14

### Ligand Screens for GPCRs

### Example 14A

### Reporter Gene Screen

Mammalian cells such as Hek293 or CHO-K1 cells are either stably transfected with a plasmid harboring the luciferase gene under the control of a cAMP dependent promoter (CRE elements) or transduced with an adenovirus harboring a luciferase gene under the control of a cAMP dependent promoter. In addition reporter constructs can be used with the luciferase gene under the control of a Ca²⁺ dependent promoter (NF-AT elements) or a promoter that is controlled by activated NF-κB. These cells, expressing the reporter construct, are then transduced with an adenovirus harboring the cDNA of a GPCR of Table 1. Forty (40) hours after transduction the cells are treated with a large collection of reference compounds comprising peptides (LOPAP, Sigma Aldrich), lipids (Biomol, TimTech), carbohydrates (Specs), natural compounds (Specs, TimTech), small chemical compounds (Tocris), commercially available screening libraries, and compounds that have been demonstrated to have binding affinity for a polypeptide comprising an amino acid sequence selected from the group consisting of the SEQ ID NOs of the GPCRs of Table 1.

Compounds, which increase luciferase activity, are considered to be agonists for a GPCR of Table 1. These compounds are screened again for verification and screened against their ability to up-regulate BAP in osteoblast progenitor cells. The compounds are also screened to verify binding to the GPCR. The binding and reporter activity assays can be performed in essentially any order to screen compounds.

In addition, cells expressing the NF-AT reporter gene can be transduced with an adenovirus harboring the cDNA encoding the α-subunit of G₁₅ or chimerical Gα subunits. G₁₅ is a promiscuous G protein of the G_{q} class that couples to many different GPCRs and as such re-directs their signaling towards the release of intracellular Ca²⁺ stores. The chimerical G alpha subunits are members of the Gₛ and G_{i/o} family by which the last 5 C-terminal residues are replaced by those of G_{αq} these chimerical G-proteins also redirect cAMP signaling to Ca²⁺ signaling.

### EXAMPLE 14B

### FLIPR Screen

Mammalian cells such as Hek293 or CHO-K1 cells are stably transfected with an expression plasmid construct harboring the cDNA of a GPCR of Table 1. Cells are seeded, grown, and selected until sufficient stable cells can be obtained. Cells are loaded with a Ca²⁺ dependent fluorophore such as Fura3 or Fura4. After washing away the excess of fluorophore the cells are screened against a large collection of reference compounds comprising peptides (LOPAP, Sigma Aldrich), lipids (Biomol, TimTech), carbohydrates (Specs), natural compounds (Specs, TimTech), small chemical compounds (Tocris), commercially available screening libraries, and compounds that have been demonstrated to have binding affinity for a polypeptide comprising an amino acid sequence selected from the group consisting of SEQ ID NOs of the GPCRs of Table 1, and a compound to the cells. Activation of the receptor is measured as an almost instantaneously increase in fluorescence due to the interaction of the fluorophore and the Ca²⁺ that is released. Compounds that increase fluorescence are considered to be agonists for the receptor they are screened against. These compounds are screened again to measure the binding to a GPCR of Table 1.

### EXAMPLE 14C

### AequoScreen

CHO cells, stably expressing Apoaequorin are stably transfected with a plasmid construct harboring the cDNA of a GPCR of Table 1. Cells are seeded, grown, and selected until sufficient stable cells can be obtained. The cells are loaded with coelenterazine, a cofactor for apoaequorin. Upon receptor activation intracellular Ca²⁺ stores are emptied and the aequorin will react with the coelenterazine in a light emitting process. The emitted light is a measure for receptor activation. The CHO cells stably expressing both the apoaequorin and the receptor are screened against a large collection of reference compounds comprising peptides (LOPAP, Sigma Aldrich), lipids (Biomol, TimTech), carbohydrates (Specs), natural compounds (Specs, TimTech), small chemical compounds (Tocris), commercially available screening libraries, and compounds that have been demonstrated to have binding affinity for a polypeptide comprising an amino acid sequence selected from the group consisting of SEQ ID NOs of the GPCRs of Table 1, by adding a compound to the cells. Activation of the receptor is measured as an almost instantaneously light flash due to the interaction of the apoaequorin, coelenterazine, and the Ca²⁺ that is released. Compounds that increase light are considered to be agonists for the receptor they are screened against. These compounds are screened again to measure the amount of binding to a GPCR of Table 1.

In addition, CHO cells stably expressing the apoaequorin gene are stably transfected with a plasmid construct harboring the cDNA encoding the α-subunit of G₁₅ or chimerical G_{α} subunits. G₁₅ is a promiscuous G protein of the G_{q} class that couples to many different GPCRs and as such redirects their signaling towards the release of intracellular Ca²⁺ stores. The chimerical G alpha subunits are members of the Gₛ and G_{i/o} family by which the last 5 C-terminal residues are replaced by those of G_{αq}, these chimerical G-proteins also redirect cAMP signaling to Ca²⁺ signaling.

### EXAMPLE 14D

### Screening for Compounds that Bind to the GPCR Polypeptides (Displacement Experiment)

Compounds are screened for binding to the GPCR of Table 1 polypeptides. The affinity of the compounds to the polypeptides is determined in a displacement experiment. In brief, the GPCR polypeptides are incubated with a labeled (radiolabeled, fluorescent labeled) ligand that is known to bind to the polypeptide and with an unlabeled compound. The displacement of the labeled ligand from the polypeptide is determined by measuring the amount of labeled ligand that is still associated with the polypeptide. The amount associated with the polypeptide is plotted against the concentration of the compound to calculate IC₅₀ values. This value reflects the binding affinity of the compound to its target, i.e. the GPCR of Table 1 polypeptides. Strong binders have an IC₅₀ in the nanomolar and even picomolar range. Compounds that have an IC₅₀ of at least 10 micromol or better (nmol to pmol) are applied in e.g. the alkaline phosphatase assay to check for their effect on osteogenesis. The GPCR of Table 1 polypeptides can be prepared in a number of ways depending on whether the assay is run on cells, cell fractions or biochemically, on purified proteins.

### EXAMPLE 14E

### Screening for Compounds that Bind to a GPCR of Table 1 (Generic GPCR Screening Assay)

When a G protein receptor becomes constitutively active, it binds to a G protein (G_{q}, Gₛ, Gᵢ, Gₒ) and stimulates the binding of GTP to the G protein. The G protein then acts as a GTPase and slowly hydrolyses the GTP to GDP, whereby the receptor, under normal conditions, becomes deactivated. However, constitutively activated receptors continue to exchange GDP to GTP. A non-hydrolyzable analog of GTP, [³⁵S]GTPγS, can be used to monitor enhanced binding to membranes which express constitutively activated receptors. It is reported that [³⁵S]GTPγS can be used to monitor G protein coupling to membranes in the absence and presence of ligand. Moreover, a preferred approach is the use of a GPCR-G protein fusion protein. The strategy to generate a GPCR of Table 1-G protein fusion protein is well known for those known in the art. Membranes expressing GPCR of Table 1-G protein fusion protein are prepared for use in the direct identification of candidate agonist compounds. Homogenized membranes with GPCR of Table 1-G protein fusion protein are transferred in a 96-well plate. A pin-tool is used to transfer a candidate compound in each well plus [³⁵S]GTPγS, followed by incubation on a shaker for 60 minutes at room temperature. The assay is stopped by spinning of the plates at 4000 RPM for 15 minutes at 22° C. The plates are then aspirated and radioactivity is then read.

### EXAMPLE 14F

### Receptor Ligand Binding Study on Cell Surface

The receptor is expressed in mammalian cells (Hek293, CHO, COS7) by adenoviral transduction of the cells (see U.S. Pat. No. 6,340,595). The cells are incubated with both labeled ligand (iodinated, tritiated, or fluorescent) and the unlabeled compound at various concentrations, ranging from 10 pM to 10 μM (3 hours at 4° C.: 25 mM HEPES, 140 mM NaCl, 1 mM CaCl₂, 5 mM MgCl₂ and 0.2% BSA, adjusted to pH 7.4). Reactions mixtures are aspirated onto PEI-treated GF/B glass filters using a cell harvester (Packard). The filters are washed twice with ice cold wash buffer (25 mM HEPES, 500 mM NaCl, 1 mM CaCl₂, 5 mM MgCl₂, adjusted to pH 7.4). Scintillant (MicroScint-10; 35 μl) is added to dried filters and the filters counted in a (Packard Topcount) scintillation counter. Data are analyzed and plotted using Prism software (GraphPad Software, San Diego, Calif.). Competition curves are analyzed and IC₅₀ values calculated. If one or more data points do not fall within the sigmoidal range of the competition curve or close to the sigmoidal range the assay is repeated and concentrations of labeled ligand and unlabeled compound adapted to have more data points close to or in the sigmoidal range of the curve.

### EXAMPLE 14G

### Receptor Ligand Binding Studies On Membrane Preparations

Membranes preparations are isolated from mammalian cells (Hek293, CHO, COS7) cells over expressing the receptor is done as follows: Medium is aspirated from the transduced cells and cells are harvested in 1×PBS by gentle scraping. Cells are pelleted (2500 rpm 5 min) and resuspended in 50 mM Tris pH 7.4 (10×10⁶ cells/ml). The cell pellet is homogenized by sonicating 3×5 sec (UP50H; sonotrode MS1; max amplitude: 140 μm; max Sonic Power Thickness: 125 W/cm²). Membrane fractions are prepared by centrifuging 20 min at maximal speed (13,000 rpm ˜15,000 to 20,000 g or rcf). The resulting pellet is resuspended in 500 μl 50 mM Tris pH 7.4 and sonicated again for 3×5 sec. The membrane fraction is isolated by centrifugation and finally resuspended in PBS. Binding competition and derivation of IC₅₀ values are determined as described above.

### EXAMPLE 14H

### Internalization Screen (1)

Activation of a GPCR-associated signal transduction pathway commonly leads to translocation of specific signal transduction molecules from the cytoplasm to the plasma membrane or from the cytoplasm to the nucleus. Norak has developed their transfluor assay based on agonist-induced translocation of receptor-β-arrestin-GFP complex from the cytosol to the plasma membrane and subsequent internalization of this complex, which occurs during receptor desensitization. A similar assay uses GFP tagged receptor instead of β-arrestin. Hek293 cells are transduced with a GPCR of Table 1 vector that translates for a GPCR of Table 1-eGFP fusion protein. 48 hours after transduction, the cells are set to fresh serum-free medium for 60 minutes and treated with a ligand for 15, 30, 60 or 120 minutes at 37° C. and 5% CO₂. After indicated exposure times, cells are washed with PBS and fixed with 5% paraformaldehyde for 20 minutes at RT. GFP fluorescence is visualized with a Zeiss microscope with a digital camera. This method aims for the identification of compounds that induce translocation of the arrestin fusion protein to the plasma membrane.

### EXAMPLE 14I

### Internalization Screen (2)

Various variations on translocation assays exists using β-arrestin and β-galactosidase enzyme complementation and BRET based assays with receptor as energy donor and β-arrestin as energy acceptor. Also the use of specific receptor antibodies labeled with pH sensitive dyes are used to detect agonist induced receptor translocation to acidic lysosomes. All of the translocation assays are used for screening for agonistic acting ligands.

### EXAMPLE 14J

### Melanophore Assay (Arena Pharmaceutical)

The melanophore assay is based on the ability of GPCRs to alter the distribution of melanin containing melanosomes in Xenopus melanophores. The distribution of the melanosomes depends on the exogenous receptor that is either G_{i/o} or G_{s/q} coupled. The distribution of the melanosomes (dispersed or aggregated) is easily detected by measuring light absorption. This type of assay is used for both agonist as well as antagonist compound screens.

### REFERENCES

Cortez-Retamozo et al. (2004). Cancer Res 64: 2853-7
Lipinsky, C A, et al. (2001). Adv Drug Deliv Rev 46: 3-26
Nakashima K and de Crombrugghe B, (2003). Trends Genet 19(8): 458-66

1. A method for identifying a compound that promotes osteogenesis in a population of vertebrate cells including osteoblast-progenitor cells, comprising
(a) contacting a compound with a polypeptide comprising an amino acid sequence selected from the group consisting of SEQ ID NO: 51-68 and 69-231; and
(b) measuring a compound-polypeptide property related to osteogenesis.

2. The method according to claim 1, wherein said polypeptide comprises SEQ ID NO: 51-68, 69-230, or 231 in an in vitro cell-free preparation.

3. The method according to claim 1, wherein said polypeptide is membrane-bound.

4. The method according to claim 3, wherein said polypeptide is present as a transmembrane cell receptor in a mammalian cell.

5. The method of claim 1, wherein said property is a binding affinity of said compound to said polypeptide.

6. The method of claim 1, wherein said property is activation of a biological pathway producing an indicator of osteogenic differentiation.

7. The method of claim 6 wherein said polypeptide comprises SEQ ID NO: 51-67 or 68.

8. The method of claim 7 and wherein said indicator is a second messenger that is cyclic AMP or Ca²⁺.

9. The method of claim 6 wherein said indicator is bone alkaline phosphatase, type-1 collagen, osteocalcin, or osteopontin.

10. The method of claim 9 wherein said indicator is bone alkaline phosphatase.

11. The method according to claim 6 wherein said indicator induces the expression of a reporter in said mammalian cell.

12. The method according to claim 11 wherein the reporter is selected from the group consisting of alkaline phosphatase, GFP, eGFP, dGFP, luciferase and β-galactosidase.

13. The method according to claim 1, wherein said compound is selected from the group consisting of compounds of a commercially available screening library and compounds that have been demonstrated to have binding affinity for a polypeptide comprising an amino acid sequence selected from the group consisting of SEQ ID NO: 51-68 and 69-231.

14. The method according to claim 13, wherein said compound is a peptide in a phage display library or an antibody fragment library.

15. The method according to claim 13, wherein said compound is an agonist of a GPCR or a NHR.

16. A pharmaceutical composition for the treatment or prevention of a condition involving an imbalance in bone homeostasis or a susceptibility to the condition, comprising a therapeutically effective amount of an expressible nucleic acid sequence encoding a polypeptide comprising an amino acid sequence selected from the group consisting of SEQ ID NO: 51-68 and 69-231.

17. The pharmaceutical composition according to claim 16, wherein said nucleic acid sequence is contained in a vector.

18. The pharmaceutical composition according to claim 1 7, wherein said vector is an adenoviral, retroviral, adeno-associated viral, lentiviral, a herpes simplex viral or a sendaiviral vector.

19. The pharmaceutical composition according to claim 18, wherein said nucleic acid sequence is selected from the group consisting of SEQ ID NO: 1-18.

20. A method of promoting osteogenic differentiation in a subject suffering or susceptible to an imbalance in bone homeostasis, comprising administering to said subject a therapeutically effective amount of an agonist of a polypeptide comprising an amino acid sequence selected from the group consisting of SEQ ID NO: 51-68 and 69-231.

21. A method according to claim 20 wherein the imbalance in bone homeostasis is due to hypocalcaemia of malignancy, Paget's disease, inflammatory bone diseases such as rheumatoid arthritis and periodontal disease, focal osteogenesis occurring during skeletal metastases, Crouzon's syndrome, rickets, opsismodysplasia, pycnodysostosis/Toulouse-Lautrec disease, osteogenesis imperfecta, or osteoporosis.

22. The method according to claim 21 for treatment or prevention of osteoporosis.

23. A pharmaceutical composition for the treatment or prevention of an imbalance in bone homeostasis or a susceptibility to the condition, comprising an effective bone alkaline phosphatase-inducing amount of a compound that is known to be an agonist for one or more of the GPCR protein receptors of SEQ ID NOs: 51, 55-60, 64-66 or 67.

24. A composition according to claim 23, wherein said compound is present as its pharmaceutically acceptable salt, hydrate, solvate, or prodrug in admixture with a pharmaceutically acceptable carrier.

25. A composition according to claim 24, further comprising labeling indicating use of said composition for the treatment or prevention of a condition involving an imbalance in bone homeostasis or a susceptibility to said condition.

26. A method for in vitro production of bone tissue comprising applying undifferentiated vertebrate cells onto a substrate to form a cellular layered article, and contacting a polynucleotide comprising an expressible nucleic acid sequence selected from the group consisting of SEQ ID NO: 1-18 with said article for a time sufficient to differentiate said undifferentiated cells into osteoblasts, thereby producing a matrix containing osteoblast cells.

27. A method according to claim 26, wherein said osteoblasts deposit sufficient calcium to form bone tissue that comprises a thickness of at least 0.5 μm on the surface of said substrate.

28. A method for producing osteoblasts for an implant comprising inducing ex vivo differentiation of inesenchymal pluripotent cells into osteoblasts by contacting a polynucleotide comprising an expressible nucleic acid sequence selected from the group consisting of SEQ ID NO: 1-18 into said pluripotent cells, and isolating the osteoblasts produced thereby.

29. A method for producing a bone tissue implant comprising mixing the osteoblasts isolated in claim 28, with a matrix-forming material to form a osteoblast composition; and applying said osteoblast composition to a synthetic graft to produce an implant.

30. A composition for the treatment of defects in bones comprising a bone-defect filling matrix containing undifferentiated vertebrate cells and a polynucleotide comprising an expressible nucleic acid selected from the group consisting of SEQ ID NO: 1-18, wherein said polynucleotide is present in said matrix at a concentration effective to induce osteoblast differentiation.

31. A composition according to claim 30, wherein a transfectable vector comprises said expressible polynucleotide.


SEQUENCE LISTING (231 sequences)

SEQ ID NO: 1 - DNA, 2403 bp, Homo sapiens
tttgcaggtgcctcaggaaccctgaagctgggctgagccatgatgctgctgccagaacccctgcagagggcctggtttcaggagactcagagtcctctgtgaaaaagcccttggagagcgccccagcagggctgcacttggctcctgtgaggaaggggctcaggggtctgggcccctccgcctgggccgggctgggagccaggcgggcggctgggctgcagcaatggaccgtgagctggcccagcccgcgtccgtgctgagcctgcctgtcgtctgtggccatgcccatcatgggctcctcggtgtacatcacggtggagctggccattgctgtgctggccatcctgggcaatgtgctggtgtgctgggccgtgtggctcaacagcaacctgcagaacgtcaccaactactttgtggtgtcactggcggcggccgacatcgcagtgggtgtgctcgccatcccctttgccatcaccatcagcaccgggttctgcgctgcctgccacggctgcctcttcattgcctgcttcgtcctggtcctcacgcagagctccatcttcagtctcctggccatcgccattgaccgctacattgccatccgcatcccgctccggtacaatggcttggtgaccggcacgagggctaagggcatcattgccatctgctgggtgctgtcgtttgccatcggcctgactcccatgctaggttggaacaactgcggtcagccaaaggagggcaagaaccactcccagggctgcggggagggccaagtggcctgtctctttgaggatgtggtccccatgaactacatggtgtacttcaacttctttgcctgtgtgctggtgcccctgctgctcatgctgggtgtctatttgcggatcttcctggcggcgcgacgacagctgaagcagatggagagccagcctctgccgggggagcgggcacggtccacactgcagaaggaggtccatgctgccaagtcactggccatcattgtggggctctttgccctctgctggctgcccctacacatcatcaactgcttcactttcttctgccccgactgcagccacgcccctctctggctcatgtacctggccatcgtcctctcccacaccaattcggttgtgaatcccttcatctacgcctaccgtatccgcgagttccgccagaccttccgcaagatcattcgcagccacgtcctgaggcagcaagaacctttcaaggcagctggcaccagtgcccgggtcttggcagctcatggcagtgacggagagcaggtcagcctccgtctcaacggccacccgccaggagtgtgggccaacggcagtgctccccaccctgagcggaggcccaatggctatgccctggggctggtgagtggagggagtgcccaagagtcccaggggaacacgggcctcccagacgtggagctccttagccatgagctcaagggagtgtgcccagagccccctggcctagatgaccccctggcccaggatggagcaggagtgtcctgatgattcatggagtttgccccttcctaagggaaggagatctttatctttctggttggcttgaccagtcacgttgggagaagagagagagtgccaggagaccctgagggcagccggttcctactttggactgagagaagggagccccaggctggagcagcatgaggcccagcaagaagggcttgggttctgaggaagcagatgtttcatgctgtgaggccttgcaccaggtgggggccacagcaccagcagcatctttgctgggcaggcccagccctccactgcagaagcatctggaagcaccaccttgtctccacagagcagcttgggcacagcagactggcctggccctgagactggggagtggctccaatagcctcctgccacccacacaccactctccctagactctcctagggttcaggagctgctgggcccagaggtgacatttgacttttttccaggaaaaatgtaagtgtgaggaaaccctttttattttattacctttcactctctggctgctgggtctgccgtcggtcctgctgctaacctggcaccagagcctctgcccggggagcctcaggcagtcctctcctgctgtcacagctgccatccacttctcagtcccagggccatctcttggagtgacaaagctgggatcaaggatagggagttgtaacagagcagtgccagagcatgggcccaggtcccaggggagaggttggggctggcaggccactggcatgtgctgagtagcgcagagctacccagtgagaggccttgtctaactgcctttccttctaaagggaatgtttttttctgagataaaataaaaacgagccacatcgtgttttaagcttgtccaaatgaaaaaaaaaaaaaaaaa

SEQ ID NO: 2 - DNA, 1528 bp, Homo sapiens
cagtgccttggtaatgaccagggctccagaaagagatgtccttgtggctgggggcccctgtgcctgacattcctcctgactctgcggtggagctgtggaagccaggcgcacaggatgcaagcagccaggcccagggaggcagcagctgcatcctcagagaggaagccaggatgccccactctgctgggggtactgcaggggtggggctggaggctgcagagcccacagccctgctcaccagggcagagcccccttcagaacccacagagatccgtccacaaaagcggaaaaaggggccagcccccaaaatgctggggaacgagctatgcagcgtgtgtggggacaaggcctcgggcttccactacaatgttctgagctgcgagggctgcaagggattcttccgccgcagcgtcatcaagggagcgcactacatctgccacagtggcggccactgccccatggacacctacatgcgtcgcaagtgccaggagtgtcggcttcgcaaatgccgtcaggctggcatgcgggaggagtgtgtcctgtcagaagaacagatccgcctgaagaaactgaagcggcaagaggaggaacaggctcatgccacatccttgccccccaggcgttcctcacccccccaaatcctgccccagctcagcccggaacaactgggcatgatcgagaagctcgtcgctgcccagcaacagtgtaaccggcgctccttttctgaccggcttcgagtcacgccttggcccatggcaccagatccccatagccgggaggcccgtcagcagcgctttgcccacttcactgagctggccatcgtctctgtgcaggagatagttgactttgctaaacagctacccggcttcctgcagctcagccgggaggaccagattgccctgctgaagacctctgcgatcgaggtgatgcttctggagacatctcggaggtacaaccctgggagtgagagtatcaccttcctcaaggatttcagttataaccgggaagactttgccaaagcagggctgcaagtggaattcatcaaccccatcttcgagttctccagggccatgaatgagctgcaactcaatgatgccgagtttgccttgctcattgctatcagcatcttctctgcagaccggcccaacgtgcaggaccagctccaggtggagaggctgcagcacacatatgtggaagccctgcatgcctacgtctccatccaccatccccatgaccgactgatgttcccacggatgctaatgaaactggtgagcctccggaccctgagcagcgtccactcagagcaagtgtttgcactgcgtctgcaggacaaaaagctcccaccgctgctctctgagatctgggatgtgcacgaatgactgttctgtccccatattttctgttttcttggccggatggctgaggcctggtggctgcctcctagaagtggaacagactgagaagggcaaacattcctgggagctgggcaaggagatcctcccgtggcattaaaagagagtcaaagggt

SEQ ID NO: 3 - DNA, 2330 bp, Homo sapiens
gaaactggatacatggtttacagcaggtcactaatgttggaaaaagtacagagtccagggaaagacttgcttgtaactttatgaattctggattttttttcctttgctttttcttaactttcactaagggttactgtagtctgatgtgtccttcccaaggccacgaaatttgacaagctgcacttttcttttgctcaatgatttctgctttaagccaaagaactgcctataatttcactaagaatgtcttctaattcagatactggggatttacaagagtctttaaagcacggacttacacctattggtgctgggcttccggaccgacacggatcccccatcccgcccgcggtcgccttgtcatgctgcccaaagtggagacggaagccctgggactggctcgatcgcatggggaacagggccagatgccggaaaacatgcaagtgtctcaatttaaaatggtgaattactcctatgatgaagatctggaagagctttgtcccgtgtgtggagataaagtgtctgggtaccattatgggctcctcacctgtgaaagctgcaagggattttttaagcgaacagtccaaaataataaaaggtacacatgtatagaaaaccagaactgccaaattgacaaaacacagagaaagcgttgtccttactgtcgttttcaaaaatgtctaagtgttggaatgaagctagaagctgtaagggccgaccgaatgcgtggaggaaggaataagtttgggccaatgtacaagagagacagggccctgaagcaacagaaaaaagccctcatccgagccaatggacttaagctagaagccatgtctcaggtgatccaagctatgccctctgacctgaccatttcctctgcaattcaaaacatccactctgcctccaaaggcctacctctgaaccatgctgccttgcctcctacagactatgacagaagtccctttgtaacatcccccattagcatgacaatgctgcacggcagcctgcaaggttaccaaacatatggccactttcctagccgggccatcaagtctgagtacccagacccctataccagctcacccgagtccataatgggctattcatatatggatagttaccagacgagctctccagcaagcatcccacatctgatactggaacttttgaagtgtgagccagatgagcctcaagtccaggctaaaatcatggcctatttgcagcaagagcaggctaaccgaagcaagcacgaaaagctgagcacctttgggcttatgtgcaaaatggcagatcaaactgtcttctccattgtcgagtgggccaggagtagtatcttcttcagagaacttaaggttgatgaccaaatgaagctgcttcagaactgctggagtgagctcttaatcctcgaccacatttaccgacaagtggtacatggaaaggaaggatccatcttcctggttactgggcaacaagtggactattccataatagcatcacaagccggagccaccctcaacaacctcatgagtcatgcacaggagttagtggcaaaacttcgttctctccagtttgatcaacgagagttcgtatgtctgaaattcttggtgctctttagtttagatgtcaaaaaccttgaaaacttccagctggtagaaggtgtccaggaacaagtcaatgccgccctgctggactacacaatgtgtaactacccgcagcagacagagaaatttggacagctacttcttcgactacccgaaatccgggccatcagtatgcaggctgaagaatacctctactacaagcacctgaatggggatgtgccctataataaccttctcattgaaatgttgcatgccaaaagagcataagttacaacccctaggagctctgctttcaaaacaaaaagagattgggggagtggggagggggaagaagaacaggaagaaaaaaagtactctgaactgctccaagtaacgctaattaaaaacttgctttaaagatattgaatttaaaaaggcataataatcaaatactaatagcaaataaatgatgtatcagggtatttgtattgcaaactgtgaatcaaagcttcacagccccagaggattccatataaaagacattgtaatggagtggattgaactcacagatggataccaacacggtcagaagaaaaacggacagaacggttcttgtatatttaaactgatctccactatgaagaaatttaggaactaatcttattaattaggcttatacagcggggatttgagcttacaggattcctccatggtaaagctgaactgaaacaattctcaagaatgcatcagctg

SEQ ID NO: 4 - DNA, 4916 bp, Homo sapiens
aaaaagtacagagtccagggaaagacttgcttgtaactttatgaattctggatttttttttttcctttgctttttcttaactttcactaagggttactgtagtctgatgtgtccttcccaaggccacgaaatttgacaagctgcacttttcttttgctcaatgatttctgctttaagccaaagaactgcctataatttcactaagaatgtcttctaattcagatactggggatttacaagagtctttaaagcacggacttacacctattgtgtctcaatttaaaatggtgaattactcctatgatgaagatctggaagagctttgtcccgtgtgtggagataaagtgtctgggtaccattatgggctcctcacctgtgaaagctgcaagggattttttaagcgaacagtccaaaataataaaaggtacacatgtatagaaaaccagaactgccaaattgacaaaacacagagaaagcgttgtccttactgtcgttttcaaaaatgtctaagtgttggaatgaagctagaagctgtaagggccgaccgaatgcgtggaggaaggaataagtttgggccaatgtacaagagagacagggccctgaagcaacagaaaaaagccctcatccgagccaatggacttaagctagaagccatgtctcaggtgatccaagctatgccctctgacctgaccatttcctctgcaattcaaaacatccactctgcctccaaaggcctacctctgaaccatgctgccttgcctcctacagactatgacagaagtccctttgtaacatcccccattagcatgacaatgccccctcacggcagcctgcaaggttaccaaacatatggccactttcctagccgggccatcaagtctgagtacccagacccctataccagctcacccgagtccataatgggctattcatatatggatagttaccagacgagctctccagcaagcatcccacatctgatactggaacttttgaagtgtgagccagatgagcctcaagtccaggctaaaatcatggcctatttgcagcaagagcaggctaaccgaagcaagcacgaaaagctgagcacctttgggcttatgtgcaaaatggcagatcaaactctcttctccattgtcgagtgggccaggagtagtatcttcttcagagaacttaaggttgatgaccaaatgaagctgcttcagaactgctggagtgagctcttaatcctcgaccacatttaccgacaagtggtacatggaaaggaaggatccatcttcctggttactgggcaacaagtggactattccataatagcatcacaagccggagccaccctcaacaacctcatgagtcatgcacaggagttagtggcaaaacttcgttctctccagtttgatcaacgagagttcgtatgtctgaaattcttggtgctctttagtttagatgtcaaaaaccttgaaaacttccagctggtagaaggtgtccaggaacaagtcaatgccgccctgctggactacacaatgtgtaactacccgcagcagacagagaaatttggacagctacttcttcgactacccgaaatccgggccatcagtatgcaggctgaagaatacctctactacaagcacctgaacggggatgtgccctataataaccttctcattgaaatgttgcatgccaaaagagcataagttacaacccctaggagctctgctttcaaaacaaaaagagattgggggagtggggagggggaagaagaacaggaagaaaaaaagtactctgaactgctccaagtaacgctaattaaaaacttgctttaaagatattgaatttaaaaaggcataataatcaaatacttaatagcaaataaatgatgtatcagggtatttgtattgcaaactgtgaatcaaaggcttcacagccccagaggattccatataaaagacattgtaatggagtggattgaactcacagatggataccaacacggtcagaagaaaaacggacagaacggttcttgtatatttaaactgatctccactatgaagaaatttaggaactaatcttattaattaggcttatacagcgggggatttgagcttacaggattcctccatggtaaagctgaactgaaacaattctcaagaatgcatcagctgtacctacaatagcccctccctcttcctttgaaggccccagcacctctgccctgtggtcaccgaatctgtactaaggacctgtgttcagccacacccagtggtagctccaccaaatcatgaacagcctaattttgagtgtctgtgtcttagacctgcaaacagctaataggaaattctattaatatgttagcttgccattttaaatatgttctgagggttgttttgtctcgtgttcatgatgttaagaaaatgcaggcagtatccctcatcttatgtaagtgtgaattaatattaagggaaatgactacaaactttcaaagcaaatgctccatagctaaagcaacttagaccttatttctgctactgttgctgaaatgtggctttggcattgttggatttcataaaaaatttctggcaggaagtcttgttagtatacatcagtctttttcatcatccaagtttgtagttcatttaaaaatacaacattaaacacattttgctaggatgtcaaatagtcacagttctaagtagttggaaacaaaattgacgcatgttaatctatgcaaagagaaaggaaaggatgaggtgatgtattgactcaaggttcattcttgctgcaattgaacatcctcaagagttgggatggaaatggtgatttttacatgtgtcctggaaagatattaaagtaattcaaatcttccccaaaggggaaaggaagagagtgatactgacctttttaagtcatagaccaaagtctgctgtagaacaaatatgggaggacaaagaatcgcaaattcttcaaatgactattatcagtattattaacatgcgatgccacaggtatgaaagtcttgccttatttcacaattttaaaaggtagctgtgcagatgtggatcaacatttgtttaaaataaagtattaatactttaaagtcaaataagatatagtgtttacattctttaggtcctgaggggcagggggatctgtgatataacaaaatagcaaaagcggtaatttccttaatgttatttttctgattggtaattatttttaacagtacttaattattctatgtcgtgagacactaaaatcaaaaacgggaatctcatttagactttaatttttttgagattatcggcggcacaatcactttgtagaaactgtaaaaaataaaagtatctcctagtcccttaattttttcataaatatttctggcttttgagtagtgtatttatattgtatatcatactttcaactgtagacaattatgatgctaatttattgtttcttggtttcacctttgtataagatatagccaagactgaagaaaccaaatatatgtgtttactgtagcatgtcttcaaattagtggaacttagttcagggacatagaagagtcttaatgaattaaaatcattcacttgattaaatgtctgtaaatcttcatcattcctactgtagtttatttaatatctattgtaaattatgtgacttgtagcttcctctggttttcaagtaaactcaacaaggtggagtcttacctggttttcctttccaagcattgtaaattgtataccaaagatattagttattacttctgtgtgtacaaagaggattattttattatgtttattaatcacctctaatactcatccacatgaagggtacacattaggtaagctgggcgttgactcatgcgcagtctcagtcacccgtgttatcttcgtggctcaaaggacaatgcaaaatcgccgatcagagctcatacccaaagcattacagagaacagcagcatcattgccctccccagctgaaaaacaagttggctagaagatacatggagaggaatggtgtggtcaacagttaatgaaacggttctatcatgcatgtgtaatgtggatggagacaattataagatttgactataactatttggagggtctttaacattgccaaaaaaacaaatatgttgatttttattttattttattttttattttaagaggcgggatcttgatctcacatgttgcccaggctggccttgaactcctgggctcaagcattcctcctgcctcagcctcccccatagctgggactaggggtgcatgccagcatacctggctacgttgactcttaaaatctatgttctcttattttaaagatacagtgctccccactgaaaattaaacctaaaaaatgtcacatattggtatgttgttaacctggtagattaaatcatgagaatgattagaaagacgggcaacacagcgggttacatccacactgctgatcacaccaacgacaggagctgataagcaagaaagcgtcacagccagcgtctgttcacccaaggttgacaagtgaagtttctctaatgttgattgttagccgatttgtaacctggcatttacttagcaactgccttatcaattacaggatttgccggtaaaagcagactcaaatataaaggtttttggcttaacttggtttattatagttgctctatgtttgtaaacagacaatctctaatgtctgattatttgtatcacagatctgcagctgccttggacttgaatccatgcaatgtttagagtgtgaagtcagttacttgttgatgttttcttactgtatcaatgaaatacatattgtcatgtcagttcttgccaggaacttctcaacaaaatggaattttttttttcagtatttcaataaatattgatatgcccagcctgataatttttaaaaaaaaaaaa

SEQ ID NO: 5 - DNA, 5054 bp, Homo sapiens
aaaaagtacagagtccagggaaagacttgcttgtaactttatgaattctggatttttttttttcctttgctttttcttaactttcactaagggttactgtagtctgatgtgtccttcccaaggccacgaaatttgacaagctgcacttttcttttgctcaatgatttctgctttaagccaaagaactgcctataatttcactaagaatgtcttctaattcagatactggggatttacaagagtctttaaagcacggacttacacctattggtgctgggcttccggaccgacacggatcccccatccccgcccgcggtcgccttgtcatgctgcccaaagtggagacggaagccctgggactggctcgatcgcatggggaacagggccagatgccggaaaacatgcaagtgtctcaatttaaaatggtgaattactcctatgatgaagatctggaagagctttgtcccgtgtgtggagataaagtgtctgggtaccattatgggctcctcacctgtgaaagctgcaagggattttttaagcgaacagtccaaaataataaaaggtacacatgtatagaaaaccagaactgccaaattgacaaaacacagagaaagcgttgtccttactgtcgttttcaaaaatgtctaagtgttggaatgaagctagaagctgtaagggccgaccgaatgcgtggaggaaggaataagtttgggccaatgtacaagagagacagggccctgaagcaacagaaaaaagccctcatccgagccaatggacttaagctagaagccatgtctcaggtgatccaagctatgccctctgacctgaccatttcctctgcaattcaaaacatccactctgcctccaaaggcctacctctgaaccatgctgccttgcctcctacagactatgacagaagtccctttgtaacatcccccattagcatgacaatgccccctcacggcagcctgcaaggttaccaaacatatggccactttcctagccgggccatcaagtctgagtacccagacccctataccagctcacccgagtccataatgggctattcatatatggatagttaccagacgagctctccagcaagcatcccacatctgatactggaacttttgaagtgtgagccagatgagcctcaagtccaggctaaaatcatggcctatttgcagcaagagcaggctaaccgaagcaagcacgaaaagctgagcacctttgggcttatgtgcaaaatggcagatcaaactctcttctccattgtcgagtgggccaggagtagtatcttcttcagagaacttaaggttgatgaccaaatgaagctgcttcagaactgctggagtgagctcttaatcctcgaccacatttaccgacaagtggtacatggaaaggaaggatccatcttcctggttactgggcaacaagtggactattccataatagcatcacaagccggagccaccctcaacaacctcatgagtcatgcacaggagttagtggcaaaacttcgttctctccagtttgatcaacgagagttcgtatgtctgaaattcttggtgctctttagtttagatgtcaaaaaccttgaaaacttccagctggtagaaggtgtccaggaacaagtcaatgccgccctgctggactacacaatgtgtaactacccgcagcagacagagaaatttggacagctacttcttcgactacccgaaatccgggccatcagtatgcaggctgaagaatacctctactacaagcacctgaacggggatgtgccctataataaccttctcattgaaatgttgcatgccaaaagagcataagttacaacccctaggagctctgctttcaaaacaaaaagagattgggggagtggggagggggaagaagaacaggaagaaaaaaagtactctgaactgctccaagtaacgctaattaaaaacttgctttaaagatattgaatttaaaaaggcataataatcaaatacttaatagcaaataaatgatgtatcagggtatttgtattgcaaactgtgaatcaaaggcttcacagccccagaggattccatataaaagacattgtaatggagtggattgaactcacagatggataccaacacggtcagaagaaaaacggacagaacggttcttgtatatttaaactgatctccactatgaagaaatttaggaactaatcttattaattaggcttatacagcgggggatttgagcttacaggattcctccatggtaaagctgaactgaaacaattctcaagaatgcatcagctgtacctacaatagcccctccctcttcctttgaaggccccagcacctctgccctgtggtcaccgaatctgtactaaggacctgtgttcagccacacccagtggtagctccaccaaatcatgaacagcctaattttgagtgtctgtgtcttagacctgcaaacagctaataggaaattctattaatatgttagcttgccattttaaatatgttctgagggttgttttgtctcgtgttcatgatgttaagaaaatgcaggcagtatccctcatcttatgtaagtgtgaattaatattaagggaaatgactacaaactttcaaagcaaatgctccatagctaaagcaacttagaccttatttctgctactgttgctgaaatgtggctttggcattgttggatttcataaaaaatttctggcaggaagtcttgttagtatacatcagtctttttcatcatccaagtttgtagttcatttaaaaatacaacattaaacacattttgctaggatgtcaaatagtcacagttctaagtagttggaaacaaaattgacgcatgttaatctatgcaaagagaaaggaaaggatgaggtgatgtattgactcaaggttcattcttgctgcaattgaacatcctcaagagttgggatggaaatggtgatttttacatgtgtcctggaaagatattaaagtaattcaaatcttccccaaaggggaaaggaagagagtgatactgacctttttaagtcatagaccaaagtctgctgtagaacaaatatgggaggacaaagaatcgcaaattcttcaaatgactattatcagtattattaacatgcgatgccacaggtatgaaagtcttgccttatttcacaattttaaaaggtagctgtgcagatgtggatcaacatttgtttaaaataaagtattaatactttaaagtcaaataagatatagtgtttacattctttaggtcctgaggggcagggggatctgtgatataacaaaatagcaaaagcggtaatttccttaatgttatttttctgattggtaattatttttaacagtacttaattattctatgtcgtgagacactaaaatcaaaaacgggaatctcatttagactttaatttttttgagattatcggcggcacaatcactttgtagaaactgtaaaaaataaaagtatctcctagtcccttaattttttcataaatatttctggcttttgagtagtgtatttatattgtatatcatactttcaactgtagacaattatgatgctaatttattgtttcttggtttcacctttgtataagatatagccaagactgaagaaaccaaatatatgtgtttactgtagcatgtcttcaaattagtggaacttagttcagggacatagaagagtcttaatgaattaaaatcattcacttgattaaatgtctgtaaatcttcatcattcctactgtagtttatttaatatctattgtaaattatgtgacttgtagcttcctctggttttcaagtaaactcaacaaggtggagtcttacctggttttcctttccaagcattgtaaattgtataccaaagatattagttattacttctgtgtgtacaaagaggattattttattatgtttattaatcacctctaatactcatccacatgaagggtacacattaggtaagctgggcgttgactcatgcgcagtctcagtcacccgtgttatcttcgtggctcaaaggacaatgcaaaatcgccgatcagagctcatacccaaagcattacagagaacagcagcatcattgccctccccagctgaaaaacaagttggctagaagatacatggagaggaatggtgtggtcaacagttaatgaaacggttctatcatgcatgtgtaatgtggatggagacaattataagatttgactataactatttggagggtctttaacattgccaaaaaaacaaatatgttgatttttattttattttattttttattttaagaggcgggatcttgatctcacatgttgcccaggctggccttgaactcctgggctcaagcattcctcctgcctcagcctcccccatagctgggactaggggtgcatgccagcatacctggctacgttgactcttaaaatctatgttctcttattttaaagatacagtgctccccactgaaaattaaacctaaaaaatgtcacatattggtatgttgttaacctggtagattaaatcatgagaatgattagaaagacgggcaacacagcgggttacatccacactgctgatcacaccaacgacaggagctgataagcaagaaagcgtcacagccagcgtctgttcacccaaggttgacaagtgaagtttctctaatgttgattgttagccgatttgtaacctggcatttacttagcaactgccttatcaattacaggatttgccggtaaaagcagactcaaatataaaggtttttggcttaacttggtttattatagttgctctatgtttgtaaacagacaatctctaatgtctgattatttgtatcacagatctgcagctgccttggacttgaatccatgcaatgtttagagtgtgaagtcagttacttgttgatgttttcttactgtatcaatgaaatacatattgtcatgtcagttcttgccaggaacttctcaacaaaatggaattttttttttcagtatttcaataaatattgatatgcccagcctgataatttttaaaaaaaaaaaa

SEQ ID NO: 6 - DNA, 1086 bp, Homo sapiens
atgaatgaatccaggtggactgaatggaggatcctgaacatgagcagtggcattgtgaatgcgtccgagcgtcactcctgcccacttggatttggccactacagtgtggtggatgtctgcatcttcgagacagtggttattgtgttgctgacatttctgattattgctgggaatctaacagttatctttgcctttcattgtgctccactgttacatcattatactaccagctatttcattcagacgatggcatatgctgatcttttcgttggagttagctgcttggttcctactctgtcacttctccactactccacaggtgtccacgagtcattaacttgccgggtttttggatatatcatctcagttctaaaaagtgtttctatggcatgtcttgcttgcatcagtgtggatcgttatcttgcaataaccaagcctctttcctacaatcaactggtcaccccttgtcgcttgagaatttgcattattttgatctggatctactcctgcctaattttcttgccttccttttttggctgggggaaacctggttaccatggtgacatttttgaatggtgtgccacgtcttggctcaccagtgcctattttactggctttattgtttgcttactttatgctcctgctgcctttgttgtctgcttcacctacttccacattttcaaaatttgccgtcagcacaccaaagagataaatgaccgaagagcccgattccctagtcatgaggtagattcttccagagagactggacacagccctgaccgtcgctacgccatggttttgtttaggataaccagtgtattttatatgctgtggctcccctatataatttactttcttctagaaagctcccgggtcttggacaatccaactctgtccttcttaacaacctggcttgcagtaagtaatagtttttgtaactgtgtaatatacagcctctccaacggcgttttccggctaggcctccgaagactgtttgagacaatgtgcacatcctgtatgtgtgtgaaggatcaggaagcacaagaacccaaacctaggaaacgggctaattcttgctccatttga

SEQ ID NO: 7 - DNA, 1382 bp, Homo sapiens
ggccttatctttccagtcgtccagcatgctctgcccaccccacgccgaggtgcactgaccatgagcctcaactcctccctcagctgcaggaaggagctgagtaatctcactgaggaggagggtggcgaagggggcgtcatcatcacccagttcatcgccatcattgtcatcaccatttttgtctgcctgggaaacctggtcatcgtggtcaccttgtacaagaagtcctacctcctcaccctcagcaacaagttcgtcttcagcctgactctgtccaacttcctgctgtccgtgttggtgctgccttttgtggtgacgagctccatccgcagggaatggatctttggtgtagtgtggtgcaacttctctgccctcctctacctgctgatcagctctgccagcatgctaaccctcggggtcattgccatcgaccgctactatgctgtcctgtaccccatggtgtaccccatgaagatcacagggaaccgggctgtgatggcacttgtctacatctggcttcactcgctcatcggctgcctgccacccctgtttggttggtcatccgtggagtttgacgagttcaaatggatgtgtgtggctgcttggcaccgggagcctggctacacggccttctggcagatctggtgtgccctcttcccctttctggtcatgctggtgtgctatggcttcatcttccgcgtggccagggtcaaggcacgcaaggtgcactgtggcacagtcgtcatcgtggaggaggatgctcagaggaccgggaggaagaactccagcacctccacctcctcttcaggcagcaggaggaatgcctttcagggtgtggtctactcggccaaccagtgcaaagccctcatcaccatcctggtggtcctcggtgccttcatggtcacctggggcccctacatggttgtcatcgcctctgaggccctctgggggaaaagctccgtctccccgagcctggagacttgggccacatggctgtcctttgccagcgctgtctgccaccccctgatctatggactctggaacaagacagttcgcaaagaactactgggcatgtgctttggggaccggtattatcgggaaccatttgtgcaacgacagaggacttccaggctcttcagcatttccaacaggatcacagacctgggcctgtccccacacctcactgcgctcatggcaggtggacagcccctggggcacagcagcagcacgggggacactggcttcagctgctcccaggactcaggtaacctgcgtgctttataagcctctcacctgtcgcgttttccctgtgttgcgtttcccccgtgtcgcgtttcccctgtgcaggctcaagagctggcggaggggcatttcccacggtg

SEQ ID NO: 8 - DNA, 2733 bp, Homo sapiens
gtaggtgcgtgcccgggactctgccgatgcgggccgcgccccctggggccaagagactccacgtcccgctcagggcaaagggtgtggggagaagcggtcacgcccctcggctggaatccgtgaggaccaggtctttttcagtgtgctcaatatggtcaccaagcctggaaacatagagtctcaccctgttgcttaggcagatggcgcgatctcggctcctgggttcaagcaattctcctgcctcagcctcctgaatagccgggattacagtcgtccagcatgctctgcccaccccacgccgaggtgcactgaccatgagcctcaactcctccctcagctgcaggaaggagctgagtaatctcactgaggaggagggtggcgaagggggcgtcatcatcacccagttcatcgccatcattgtcatcaccatttttgtctgcctgggaaacctggtcatcgtggtcaccttgtacaagaagtcctacctcctcaccctcagcaacaagttcgtcttcagcctgactctgtccaacttcctgctgtccgtgttggtgctgccttttgtggtgacgagctccatccgcagggaatggatctttggtgtagtgtggtgcaacttctctgccctcctctacctgctgatcagctctgccagcatgctaaccctcggggtcattgccatcgaccgctactatgctgtcctgtaccccatggtgtaccccatgaagatcacagggaaccgggctgtgatggcacttgtctacatctggcttcactcgctcatcggctgcctgccacccctgtttggttggtcatccgtggagtttgacgagttcaaatggatgtgtgtggctgcttggcaccgggagcctggctacacggccttctggcagatctggtgtgccctcttcccctttctggtcatgctggtgtgctatggcttcatcttccgcgtggccagggtcaaggcacgcaaggtgcactgtggcacagtcgtcatcgtggaggaggatgctcagaggaccgggaggaagaactccagcacctccacctcctcttcaggcagcaggaggaatgcctttcagggtgtggtctactcggccaaccagtgcaaagccctcatcaccatcctggtggtcctcggtgccttcatggtcacctggggcccctacatggttgtcatcgcctctgaggccctctgggggaaaagctccgtctccccgagcctggagacttgggccacatggctgtcctttgccagcgctgtctgccaccccctgatctatggactctggaacaagacagttcgcaaagaactactgggcatgtgctttggggaccggtattatcgggaaccatttgtgcaacgacagaggacttccaggctcttcagcatttccaacaggatcacagacctgggcctgtccccacacctcactgcgctcatggcaggtggacagcccctggggcacagcagcagcacgggggacactggcttcagctgctcccaggactcagggacagatatgatgctgcttgaggactacacgtctgatgacaaccctccctctcactgcacttgcccacccaagagaaggagctcggtgacatttgaggatgaagtggaacaaatcaaagaagctgccaagaactcgattcttcatgtgaaagctgaagtacacaagtccttggacagttacgcagcaagcttggccaaagccattgaggccgaagccaaaatcaacttatttggggaggaggctttgccaggggtcttggttacagcacggactgtcccggggggcggcttcgggggccgccgaggcagcagaactcttgtgagccagaggctgcagttgcagagcatcgaagaaggagatgttttagctgccgagcagagatgagggcctcagggtgccgtggggctgcagcctgagaggctggcccggggaggagttcccatcaccgcctgtgccgcggccttgggagcatgtcactgtgtacagctggccacacacagggaaggagcagcatctggtatgcagccaccaggacaaggactgaaaataatgtctacagtccacagcttcagcatttccagagaccacatgtgagcttcttttaggtcccagtgatgggaccagaagcatctaaagcaaaaaaaaaaccaaaaaaaaatctagagatgtggctgtggctttttgggaggtggggcatgggaggaccagagacgaagggcttggaaggagacccccacatgcatcatttcctcctcttcacagtgtgctgggagtccagccgtgcactgtgccagatgcctcaggaggagaaccctccccagtgtactgtgaaggatgacacagcacttcttcctaatgacacgcgaccgtcctggtgcctctacatggttgatgcgggcagtgtgggaccctcagttctaggactggtccgcagagaaagcacccaggagcagagcgcttcggagcggtcctcagtggcgccacctgctggtgctaatagggacagccacaggcctcttgcagactggcccaccctgcctacttccctactgacaagttctttggtatttcaaaagggagaaaccactataaaagacaagaggaagggcaggtactaggtgtttcatttccagaattaaaaaaaaaatccatttaaaaaaaaaaaaaaaaaaaaaaaaaaaaaaaaaaaaaaaaaaaaaaaaaaaaaaaaaaa

SEQ ID NO: 9 - DNA, 2299 bp, Homo sapiens
gatgactgctccctggccttttggttccgagggagtgcagggcagcagtgtgaagacctgtttacgtccactccccactccccggggctggcttcgggacgaattcactgtggtattaacagagcccgggacagagcctggcagaaggcagatggatgctcgtgcccgtcggctggatgaatgaaagtcgtccagcatgctctgcccaccccacgccgaggtgcactgaccatgagcctcaactcctccctcagctgcaggaaggagctgagtaatctcactgaggaggagggtggcgaagggggcgtcatcatcacccagttcatcgccatcattgtcatcaccatttttgtctgcctgggaaacctggtcatcgtggtcaccttgtacaagaagtcctacctcctcaccctcagcaacaagttcgtcttcagcctgactctgtccaacttcctgctgtccgtgttggtgctgccttttgtggtgacgagctccatccgcagggaatggatctttggtgtagtgtggtgcaacttctctgccctcctctacctgctgatcagctctgccagcatgctaaccctcggggtcattgccatcgaccgctactatgctgtcctgtaccccatggtgtaccccatgaagatcacagggaaccgggctgtgatggcacttgtctacatctggcttcactcgctcatcggctgcctgccacccctgtttggttggtcatccgtggagtttgacgagttcaaatggatgtgtgtggctgcttggcaccgggagcctggctacacggccttctggcagatctggtgtgccctcttcccctttctggtcatgctggtgtgctatggcttcatcttccgcgtggccagggtcaaggcacgcaaggggcactgtggcacagtcgtcatcgtggaggaggatgctcagaggaccgggaggaagaactccagcacctccacctcctcttcaggcagcaggaggaatgcctttcagggtgtggtctactcggccaaccagtgcaaagccctcatcaccatcctggtggtcctcggtgccttcatggtcacctggggcccctacatggttgtcatcgcctctgaggccctctgggggaaaagctccgtctccccgagcctggagacttgggccacatggctgtcctttgccagcgctgtctgccaccccctgatctatggactctggaacaagacagttcgcaaagaactactgggcatgtgctttggggaccggtattatcgggaaccatttgtgcaacgacagaggacttccaggctcttcagcatttccaacaggatcacagacctgggcctgtccccacacctcactgcgctcatggcaggtggacagcccctggggcacagcagcagcacgggggacactggcttcagctgctcccaggactcagggacagatatgatgctgcttgaggactacacgtctgatgacaaccctccctctcactgcacttgcccacccaagagaaggagctcggtgacatttgaggatgaagtggaacaaatcaaagaagctgccaagaactcgattcttcatgtgaaagctgaagtacacaagtccttggacagttacgcagcaagcttggccaaagccattgaggccgaagccaaaatcaacttatttggggaggaggctttgccaggggtcttggttacagcacggactgtcccggggggcggcttcgggggccgccgaggcagcagaactcttgtgagccagaggctgcagttgcagagcatcgaagaaggagatgttttagctgccgagcagagatgagggcctcagggtgccgtggggctgcagcctgagaggctggcccggggaggagttcccatcaccgcctgtgccgcggccttgggagcatgtcactgtgtacagctggccacacacagggaaggagcagcatctggtatgcagccaccaggacaaggactgaaaataatgtctacagtccacagcttcagcatttccagagaccacatgtgagcttcttttaggtcccagtgatgggaccagaagcatctaaagccaaaaaaaaaccaaaaaaaaaaaaaaaaaaaaagggggggcggcggaaaggggggcccgtacacgcggggaattaaatacgggacgggaacagggagggagaacaagggaattcgagaacgaggttatgagagcgcgggaccacagggggggcccggtcaccattggcccaaagggagggcagaataggcggcgaaggtggcggcgttacacacaggcgggactg

SEQ ID NO: 10 - DNA, 1787 bp, Homo sapiens
ttcttgacttgatgcaggcacagatttatcaagctcctcagtcaacaaacacatcaccggaagaaacatggaaggaaaggaattttaaaaggaaataccaatctctgtgcaaacaaagccttgtatattcatgtttgcaccaatctactgtgagatttatgaagaaaaacaaattgcggacaactctctatgtacacttacaaatgcctcagttgatgcttgtgggctgtttgtcagcgttctgtgataatgaacacatggacttctgtttattaaattcagttgacccctttagccaattgccaggagcctggatttttacttccaactgctgatatctgtgtaaaaattgatctacatccaccctttaaaagcattgatgaattaattagaactttagacaacaaagaaaaattgaaaaagaattctcagtaaaagcgaattcgatgttcaaaacaaactacaaagagacaagacttctctgtttactttctaagaactaatataattgctaccttaaaaaggaaaaaatgaacagcacatgtattgaagaacagcatgacctggatcactatttgtttcccattgtttacatctttgtgattatagtcagcattccagccaatattggatctctgtgtgtgtctttcctgcaagcaaagaaggaaagtgaactaggaatttacctcttcagtttgtcactatcagatttactctatgcattaactctccctttatggattgattatacttggaataaagacaactggactttctctcctgccttgtgcaaagggagtgcttttctcatgtacatgaatttttacagcagcacagcattcctcacctgcattgccgttgatcggtatttggctgttgtctaccctttgaagttttttttcctaaggacaagaagatttgcactcatggtcagcctgtccatctggatattggaaaccatcttcaatgctgtcatgttgtgggaagatgaaacagttgttgaatattgcgatgccgaaaagtctaattttactttatgctatgacaaataccctttagagaaatggcaaatcaacctcaacttgttcaggacgtgtacaggctatgcaatacctttggtcaccatcctgatctgcaaccggaaagtctaccaagctgtgcggcacaataaagccacggaaaacaaggaaaagaagagaatcataaaactacttgtcagcatcacagttacttttgtcttatgctttactccctttcatgtgatgttgctgattcgctgcattttagagcatgctgtgaacttcgaagaccacagcaattctgggaagcgaacttacacaatgtatagaatcacggttgcattaacaagtttaaattgtgttgctgatccaattctgtactgttttgtaaccgaaacaggaagatatgatatgtggaatatattaaaattctgcactgggaggtgtaatacatcacaaagacaaagaaaacgcatactttctgtgtctacaaaagatactatggaattagaggtccttgagtagaaccaaggatgttttgaagggaagggaagtttaagttatgcattattatatcatcaagattacattttgaaaaggaaatctagcatgtgaggggactaagtgttctcagagtgatgttttaatccagtccaataaaaatatcttaaaactgcattgtacagctccctccctgcgttttattaaatgatgtatattaaacaaagatcaataaaaaaaaaaaaaaaaaaaaaaaaaaaaaaaaaa

SEQ ID NO: 11 - DNA, 5253 bp, Homo sapiens
aatcggggctttaagtccttgattaggagagtgtgagagctttggtcccaactggctgtgcctataggcttgtcactaggagaacatttgtgttaattgcactgtgctctgtcaaggaaactttgatttatagctggggtgcacaaataatggttgccggtcgcacatggattcggtagaactttgccttcctgaatctttttccctgcactacgaggaagagcttctctgcagaatgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccatataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcgtttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtctgactaaaagctccctgggccttcccatccttcatgttgaaaaagggaaaataaacccaagagtgatgtcgaagaaacttagagtttagttaacaacatcaaaaatcaacagactgcactgataatttagcagcaagactatgaagcagctttcagattcctccataggttcctgatgagtttctttctactttctccatcatcttctttcctctttcttcccacatttctctttctctttattttttctccttttcttctttcacctcccttatttctttgcttctttcattcctagttcccattctcctttattttcttcccgtctgcctgccttctttcttttctttacctactctcattcctctcttttctcatccttccccttttttctaaatttgaaatagctttagtttaaaaaaaaatcctcccttccccctttcctttccctttctttcctttttccctttccttttccctttcctttcctttcctcttgaccttctttccatctttctttttcttccttctgctgctgaacttttaaaagaggtctctaactgaagagagatggaagccagccctgccaaaggatggagatccataatatggatgccagtgaacttattgtgaaccatactgtccccaatgactaaggaatcaaagagagagaaccaacgttcctaaaagtacagtgcaacatatacaaattgactgagtgcagtattagatttcatgggagcagcctctaattagacaacttaagcaacgttgcatcggctgcttcttatcattgcttttccatctagatcagttacagccatttgattccttaattgttttttcaagtcttccaggtatttgttagtttagctactatgtaactttttcagggaatagtttaagctttattcattcatgcaatactaaagagaaataagaatactgcaattttgtgctggctttgaacaattacgaacaataatgaaggacaaatgaatcctgaaggaagatttttaaaaatgttttgtttcttcttacaaatggagatttttttgtaccagctttaccacttttcagccatttattaatatgggaatttaacttactcaagcaatagttgaagggaaggtgcatattatcacggatgcaatttatgttgtgtgccagtctggtcccaaacatcaatttcttaacatgagctccagtttacctaaatgttcactgacacaaaggatgagattacacctacagtgactctgagtagtcacatatataagcactgcacatgagatatagatccgtagaattgtcaggagtgcacctctctacttgggaggtacaattgccatatgatttctagctgccatggtggttaggaatgtgatactgcctgtttgcaaagtcacagaccttgcctcagaaggagctgtgagccagtattcatttaagaggcaataaggcaaatgccagaattaaaaaaaaaaatcatcaaagacagaaaatgcctgaccaaattctaaaacctaatccatataagtttattcatttaggaatgttcgtttaaattaatctgcagtttttaccaagagctaagccaatatatgtgcttttcaaccagtattgtcacagcatgaaagtcaagtcaggttccagactgttaagaggtgtaatctaatgaagaaatcaattagatgccccgaaatctacagtcgctgaataaccaataaacagtaacctccatcaaatgctataccaatggaccagtgttagtagctgctccctgtattatgtgaacagtcttattctatgtacacagatgtaattaaaattgtaatcctaacaaacaaaagaaatgtagttcagcttttcaatgtttcatgtttgctgtgcttttctgaattttatgttgcattcaaagactgttgtcttgttcttgtggtgtttggattcttgtggtgtgtgcttttagacacagggtagaattagagacaatattggatgtacaattcctcaggagactacagtagtatattctattccttaccagtaataaggttcttcctaataataattaagagattgaaactccaaacaagtattcattatgaacagatacacatcaaaatcataataatattttcaaaacaaggaataatttctctaatggtttattatagaataccaatgtatagcttagaaataaaactttgaatatttcaagaatatagataagtctaatttttaaatgctgtatatatggctttcactcaatcatctctcagatgttgttattaactcgctctgtgttgttgcaaaactttttggtgcagattcgtttccaaaactattgctactttgtgtgctttaaacaaaataccttgggttgatgaaacatcaacccagtgctaggaatactgtgtatctatcattagctatatgggactatattgtagattgtggtttctcagtagagaagtgactgtagtgtgattctagataaatcatcattagcaattcattcagatggtcaataacttgaaatttatagctgtgataggagttcagaaattggcacatccctttaaaaataacaacagaaaatacaactcctgggaaaaaaggtgctgattctataagattatttatatatgtaagtgtttaaaaagattattttccagaaagtttgtgcagggtttaagttgctactattcaactacactatatataaataaaatatatacaatatatacattgttttcactgtatcacattaaagtacttgggcttcagaagtaagagccaaccaactgaaaacctgagatggagatatgttcaaagaatgagatacaattttttagttttcagtttaagtaactctcagcattacaaaagagtaagtatctcacaaataggaaataaaactaaaacgtggatttaaaaagaactgcacgggctttagggtaaatgctcatcttaaacctcactagagggaagtcttctcaagtttcaagcaagaccatttacttaatgtgaagttttggaaagttataaaggtgtatgttttagccatatgattttaattttaattttgcttcttttaggttcgttcttatttaaagcaatatgattgtgtgactccttgtagttacacttgtgtttcaatcagatcagattgttgtatttattccactattttgcatttaaatgataacataaaagatataaaaaatttaaaactgctatttttcttatagaagagaaaatgggtgttggtgattgtattttaattatttaagcgtctctgtttacctgcctaggaaaacattttatggcagtcttatgtgcaaagatcgtaaaaggacaaaaaatttaaactgcttataataatccaggagttgcattatagccagtagtaaaaataataataataataataaaaccatgtctatagctgtagatgggcttcacatctgtaaagcaatcaattgtatatttttgtgatgtgtaccatactgtgtgctccagcaaatgtccatttgtgtaaatgtatttattttatattgtatatattgttaaatgcaaaaaggagatatgattctgtaactccaatcagttcagatgtgtaactcaaattattatgcctttcaggatgatggtagagcaatattaaacaagcttccacttttgactgctaaaaaaaaaaaaaaaaa

SEQ ID NO: 12 - DNA, 5369 bp, Homo sapiens
gtgtaagatgtgcaatgttcctgcatgggggttttgtagactttcatagccaaagaaaccggcttcggcttctttaaaatccccgacgactcacctgattaacctgctgcagttctgaccctgccaagagctgacaatttactggttcatcaatgaaacaatattaaattatgaagatgtaaggaaaaaatcctacgctaacactgtcgcagtttgaaagctttaaaaagttgaagtctattggaaaccttcttataagaaaccttcctagtcagagaattcaactttcttcttttcttcattcatgaaaatgcagatccactgagaaagggaataaggcttctctgcagaatgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccatataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcgtttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtctgactaaaagctccctgggccttcccatccttcatgttgaaaaagggaaaataaacccaagagtgatgtcgaagaaacttagagtttagttaacaacatcaaaaatcaacagactgcactgataatttagcagcaagactatgaagcagctttcagattcctccataggttcctgatgagtttctttctactttctccatcatcttctttcctctttcttcccacatttctctttctctttattttttctccttttcttctttcacctcccttatttctttgcttctttcattcctagttcccattctcctttattttcttcccgtctgcctgccttctttcttttctttacctactctcattcctctcttttctcatccttccccttttttctaaatttgaaatagctttagtttaaaaaaaaatcctcccttccccctttcctttccctttctttcctttttccctttccttttccctttcctttcctttcctcttgaccttctttccatctttctttttcttccttctgctgctgaacttttaaaagaggtctctaactgaagagagatggaagccagccctgccaaaggatggagatccataatatggatgccagtgaacttattgtgaaccatactgtccccaatgactaaggaatcaaagagagagaaccaacgttcctaaaagtacagtgcaacatatacaaattgactgagtgcagtattagatttcatgggagcagcctctaattagacaacttaagcaacgttgcatcggctgcttcttatcattgcttttccatctagatcagttacagccatttgattccttaattgttttttcaagtcttccaggtatttgttagtttagctactatgtaactttttcagggaatagtttaagctttattcattcatgcaatactaaagagaaataagaatactgcaattttgtgctggctttgaacaattacgaacaataatgaaggacaaatgaatcctgaaggaagatttttaaaaatgttttgtttcttcttacaaatggagatttttttgtaccagctttaccacttttcagccatttattaatatgggaatttaacttactcaagcaatagttgaagggaaggtgcatattatcacggatgcaatttatgttgtgtgccagtctggtcccaaacatcaatttcttaacatgagctccagtttacctaaatgttcactgacacaaaggatgagattacacctacagtgactctgagtagtcacatatataagcactgcacatgagatatagatccgtagaattgtcaggagtgcacctctctacttgggaggtacaattgccatatgatttctagctgccatggtggttaggaatgtgatactgcctgtttgcaaagtcacagaccttgcctcagaaggagctgtgagccagtattcatttaagaggcaataaggcaaatgccagaattaaaaaaaaaaatcatcaaagacagaaaatgcctgaccaaattctaaaacctaatccatataagtttattcatttaggaatgttcgtttaaattaatctgcagtttttaccaagagctaagccaatatatgtgcttttcaaccagtattgtcacagcatgaaagtcaagtcaggttccagactgttaagaggtgtaatctaatgaagaaatcaattagatgccccgaaatctacagtcgctgaataaccaataaacagtaacctccatcaaatgctataccaatggaccagtgttagtagctgctccctgtattatgtgaacagtcttattctatgtacacagatgtaattaaaattgtaatcctaacaaacaaaagaaatgtagttcagcttttcaatgtttcatgtttgctgtgcttttctgaattttatgttgcattcaaagactgttgtcttgttcttgtggtgtttggattcttgtggtgtgtgcttttagacacagggtagaattagagacaatattggatgtacaattcctcaggagactacagtagtatattctattccttaccagtaataaggttcttcctaataataattaagagattgaaactccaaacaagtattcattatgaacagatacacatcaaaatcataataatattttcaaaacaaggaataatttctctaatggtttattatagaataccaatgtatagcttagaaataaaactttgaatatttcaagaatatagataagtctaatttttaaatgctgtatatatggctttcactcaatcatctctcagatgttgttattaactcgctctgtgttgttgcaaaactttttggtgcagattcgtttccaaaactattgctactttgtgtgctttaaacaaaataccttgggttgatgaaacatcaacccagtgctaggaatactgtgtatctatcattagctatatgggactatattgtagattgtggtttctcagtagagaagtgactgtagtgtgattctagataaatcatcattagcaattcattcagatggtcaataacttgaaatttatagctgtgataggagttcagaaattggcacatccctttaaaaataacaacagaaaatacaactcctgggaaaaaaggtgctgattctataagattatttatatatgtaagtgtttaaaaagattattttccagaaagtttgtgcagggtttaagttgctactattcaactacactatatataaataaaatatatacaatatatacattgttttcactgtatcacattaaagtacttgggcttcagaagtaagagccaaccaactgaaaacctgagatggagatatgttcaaagaatgagatacaattttttagttttcagtttaagtaactctcagcattacaaaagagtaagtatctcacaaataggaaataaaactaaaacgtggatttaaaaagaactgcacgggctttagggtaaatgctcatcttaaacctcactagagggaagtcttctcaagtttcaagcaagaccatttacttaatgtgaagttttggaaagttataaaggtgtatgttttagccatatgattttaattttaattttgcttcttttaggttcgttcttatttaaagcaatatgattgtgtgactccttgtagttacacttgtgtttcaatcagatcagattgttgtatttattccactattttgcatttaaatgataacataaaagatataaaaaatttaaaactgctatttttcttatagaagagaaaatgggtgttggtgattgtattttaattatttaagcgtctctgtttacctgcctaggaaaacattttatggcagtcttatgtgcaaagatcgtaaaaggacaaaaaatttaaactgcttataataatccaggagttgcattatagccagtagtaaaaataataataataataataaaaccatgtctatagctgtagatgggcttcacatctgtaaagcaatcaattgtatatttttgtgatgtgtaccatactgtgtgctccagcaaatgtccatttgtgtaaatgtatttattttatattgtatatattgttaaatgcaaaaaggagatatgattctgtaactccaatcagttcagatgtgtaactcaaattattatgcctttcaggatgatggtagagcaatattaaacaagcttccacttttgactgctaaaaaaaaaaaaaaaaa

SEQ ID NO: 13 - DNA, 5251 bp, Homo sapiens
gtgtaagatgtgcaatgttcctgcatgggggttttgtagactttcatagccaaagaaaccggcttcggcttctttaaaatccccgacgactcacctgattaacctgctgcagttctgaccctgccaagagctgacaatttactggttcatcaatgaaacaatattaaattatgaagatgtaaggaaaaaatcctacgctaacactgtcgcagtttgaaaggcttctctgcagaatgtcaaacaaagatcgacacattgattccagctgttcgtccttcatcaagacggaaccttccagcccagcctccctgacggacagcgtcaaccaccacagccctggtggctcttcagacgccagtgggagctacagttcaaccatgaatggccatcagaacggacttgactcgccacctctctacccttctgctcctatcctgggaggtagtgggcctgtcaggaaactgtatgatgactgctccagcaccattgttgaagatccccagaccaagtgtgaatacatgctcaactcgatgcccaagagactgtgtttagtgtgtggtgacatcgcttctgggtaccactatggggtagcatcatgtgaagcctgcaaggcattcttcaagaggacaattcaaggcaatatagaatacagctgccctgccacgaatgaatgtgaaatcacaaagcgcagacgtaaatcctgccaggcttgccgcttcatgaagtgtttaaaagtgggcatgctgaaagaaggggtgcgtcttgacagagtacgtggaggtcggcagaagtacaagcgcaggatagatgcggagaacagcccatacctgaaccctcagctggttcagccagccaaaaagccatataacaagattgtctcacatttgttggtggctgaaccggagaagatctatgccatgcctgaccctactgtccccgacagtgacatcaaagccctcactacactgtgtgacttggccgaccgagagttggtggttatcattggatgggcgaagcatattccaggcttctccacgctgtccctggcggaccagatgagccttctgcagagtgcttggatggaaattttgatccttggtgtcgtataccggtctctttcgtttgaggatgaacttgtctatgcagacgattatataatggacgaagaccagtccaaattagcaggccttcttgatctaaataatgctatcctgcagctggtaaagaaatacaagagcatgaagctggaaaaagaagaatttgtcaccctcaaagctatagctcttgctaattcagactccatgcacatagaagatgttgaagccgttcagaagcttcaggatgtcttacatgaagcgctgcaggattatgaagctggccagcacatggaagaccctcgtcgagctggcaagatgctgatgacactgccactcctgaggcagacctctaccaaggccgtgcagcatttctacaacatcaaactagaaggcaaagtcccaatgcacaaactttttttggaaatgttggaggccaaggtctgactaaaagctccctgggccttcccatccttcatgttgaaaaagggaaaataaacccaagagtgatgtcgaagaaacttagagtttagttaacaacatcaaaaatcaacagactgcactgataatttagcagcaagactatgaagcagctttcagattcctccataggttcctgatgagtttctttctactttctccatcatcttctttcctctttcttcccacatttctctttctctttattttttctccttttcttctttcacctcccttatttctttgcttctttcattcctagttcccattctcctttattttcttcccgtctgcctgccttctttcttttctttacctactctcattcctctcttttctcatccttccccttttttctaaatttgaaatagctttagtttaaaaaaaaatcctcccttccccctttcctttccctttctttcctttttccctttccttttccctttcctttcctttcctcttgaccttctttccatctttctttttcttccttctgctgctgaacttttaaaagaggtctctaactgaagagagatggaagccagccctgccaaaggatggagatccataatatggatgccagtgaacttattgtgaaccatactgtccccaatgactaaggaatcaaagagagagaaccaacgttcctaaaagtacagtgcaacatatacaaattgactgagtgcagtattagatttcatgggagcagcctctaattagacaacttaagcaacgttgcatcggctgcttcttatcattgcttttccatctagatcagttacagccatttgattccttaattgttttttcaagtcttccaggtatttgttagtttagctactatgtaactttttcagggaatagtttaagctttattcattcatgcaatactaaagagaaataagaatactgcaattttgtgctggctttgaacaattacgaacaataatgaaggacaaatgaatcctgaaggaagatttttaaaaatgttttgtttcttcttacaaatggagatttttttgtaccagctttaccacttttcagccatttattaatatgggaatttaacttactcaagcaatagttgaagggaaggtgcatattatcacggatgcaatttatgttgtgtgccagtctggtcccaaacatcaatttcttaacatgagctccagtttacctaaatgttcactgacacaaaggatgagattacacctacagtgactctgagtagtcacatatataagcactgcacatgagatatagatccgtagaattgtcaggagtgcacctctctacttgggaggtacaattgccatatgatttctagctgccatggtggttaggaatgtgatactgcctgtttgcaaagtcacagaccttgcctcagaaggagctgtgagccagtattcatttaagaggcaataaggcaaatgccagaattaaaaaaaaaaatcatcaaagacagaaaatgcctgaccaaattctaaaacctaatccatataagtttattcatttaggaatgttcgtttaaattaatctgcagtttttaccaagagctaagccaatatatgtgcttttcaaccagtattgtcacagcatgaaagtcaagtcaggttccagactgttaagaggtgtaatctaatgaagaaatcaattagatgccccgaaatctacagtcgctgaataaccaataaacagtaacctccatcaaatgctataccaatggaccagtgttagtagctgctccctgtattatgtgaacagtcttattctatgtacacagatgtaattaaaattgtaatcctaacaaacaaaagaaatgtagttcagcttttcaatgtttcatgtttgctgtgcttttctgaattttatgttgcattcaaagactgttgtcttgttcttgtggtgtttggattcttgtggtgtgtgcttttagacacagggtagaattagagacaatattggatgtacaattcctcaggagactacagtagtatattctattccttaccagtaataaggttcttcctaataataattaagagattgaaactccaaacaagtattcattatgaacagatacacatcaaaatcataataatattttcaaaacaaggaataatttctctaatggtttattatagaataccaatgtatagcttagaaataaaactttgaatatttcaagaatatagataagtctaatttttaaatgctgtatatatggctttcactcaatcatctctcagatgttgttattaactcgctctgtgttgttgcaaaactttttggtgcagattcgtttccaaaactattgctactttgtgtgctttaaacaaaataccttgggttgatgaaacatcaacccagtgctaggaatactgtgtatctatcattagctatatgggactatattgtagattgtggtttctcagtagagaagtgactgtagtgtgattctagataaatcatcattagcaattcattcagatggtcaataacttgaaatttatagctgtgataggagttcagaaattggcacatccctttaaaaataacaacagaaaatacaactcctgggaaaaaaggtgctgattctataagattatttatatatgtaagtgtttaaaaagattattttccagaaagtttgtgcagggtttaagttgctactattcaactacactatatataaataaaatatatacaatatatacattgttttcactgtatcacattaaagtacttgggcttcagaagtaagagccaaccaactgaaaacctgagatggagatatgttcaaagaatgagatacaattttttagttttcagtttaagtaactctcagcattacaaaagagtaagtatctcacaaataggaaataaaactaaaacgtggatttaaaaagaactgcacgggctttagggtaaatgctcatcttaaacctcactagagggaagtcttctcaagtttcaagcaagaccatttacttaatgtgaagttttggaaagttataaaggtgtatgttttagccatatgattttaattttaattttgcttcttttaggttcgttcttatttaaagcaatatgattgtgtgactccttgtagttacacttgtgtttcaatcagatcagattgttgtatttattccactattttgcatttaaatgataacataaaagatataaaaaatttaaaactgctatttttcttatagaagagaaaatgggtgttggtgattgtattttaattatttaagcgtctctgtttacctgcctaggaaaacattttatggcagtcttatgtgcaaagatcgtaaaaggacaaaaaatttaaactgcttataataatccaggagttgcattatagccagtagtaaaaataataataataataataaaaccatgtctatagctgtagatgggcttcacatctgtaaagcaatcaattgtatatttttgtgatgtgtaccatactgtgtgctccagcaaatgtccatttgtgtaaatgtatttattttatattgtatatattgttaaatgcaaaaaggagatatgattctgtaactccaatcagttcagatgtgtaactcaaattattatgcctttcaggatgatggtagagcaatattaaacaagcttccacttttgactgctaaaaaaaaaaaaaaaaa

SEQ ID NO: 14 - DNA, 1005 bp, Homo sapiens
atgaatgaagacctgaaggtcaatttaagcgggctgcctcgggattatttagatgccgctgctgcggagaacatctcggctgctgtctcctcccgggttcctgccgtagagccagagcctgagctcgtagtcaacccctgggacattgtcttgtgtacctcgggaaccctcatctcctgtgaaaatgccattgtggtccttatcatcttccacaaccccagcctgcgagcacccatgttcctgctaataggcagcctggctcttgcagacctgctggccggcattggactcatcaccaattttgtttttgcctacctgcttcagtcagaagccaccaagctggtcacgatcggcctcattgtcgcctctttctctgcctctgtctgcagcttgctggctatcactgttgaccgctacctctcactgtactacgctctgacgtaccattcggagaggacggtcacgtttacctatgtcatgctcgtcatgctctgggggacctccatctgcctggggctgctgcccgtcatgggctggaactgcctccgagacgagtccacctgcagcgtggtcagaccgctcaccaagaacaacgcggccatcctctcggtgtccttcctcttcatgtttgcgctcatgcttcagctctacatccagatctgtaagattgtgatgaggcacgcccatcagatagccctgcagcaccacttcctggccacgtcgcactatgtgaccacccggaaaggggtctccaccctggctatcatcctggggacgtttgctgcttgctggatgcctttcaccctctattccttgatagcggattacacctacccctccatctatacctacgccaccctcctgcccgccacctacaattccatcatcaaccctgtcatatatgctttcagaaaccaagagatccagaaagcgctctgtctcatttgctgcggctgcatcccgtccagtctcgcccagagagcgcgctcgcccagtgatgtgtag

SEQ ID NO: 15 - DNA, 978 bp, Homo sapiens
atgaattcctcatttcacctgcatttcttggatctcaacctgaatgccacagagggcaacctttcaggacccaatgtcaaaaacaagtcttcaccatgtgaagacatgggcattgctgtggaggtgtttctcactctgggtgtcatcagcctcttggagaacatcttggtcataggggccatagtgaagaacaaaaacctgcactcccccatgtacttcttcgtgtgcagcctggcagtggcggacatgctggtgagcatgtccagtgcctgggagaccatcaccatctacctactcaacaacaagcacctagtgatagcagacgcctttgtgcgccacattgacaatgtgtttgactccatgatctgcatttccgtggtggcatccatgtgcagcttactggccattgcagtggataggtacgtcaccatcttctacgccctgcgctaccaccacatcatgacggcgaggcgctcaggggccatcatcgccggcatctgggctttctgcacgggctgcggcattgtcttcatcctgtactcagaatccacctacgtcatcctgtgcctcatctccatgttcttcgctatgctgttcctcctggtgtctctgtacatacacatgttcctcctggcgcggactcacgtcaagcggatcgcggctctgcccggggccagctctgcgcggcagaggaccagcatgcagggcgcggtcaccgtcaccatgctgctgggcgtgtttaccgtgtgctgggccccgttcttccttcatctcactttaatgctttcttgccctcagaacctctactgctctcgcttcatgtctcacttcaatatgtacctcatactcatcatgtgtaattccgtgatggaccctctcatatatgccttccgcagccaagagatgcggaagacctttaaggagattatttgctgccgtggtttcaggatcgcctgcagctttcccagaagggattaa

SEQ ID NO: 16 - DNA, 1809 bp, Homo sapiens
agaagatcctgggttctgtgcatccgtctgtctgaccatccctctcaatcttccctgcccaggactggccatactgccaccgcacacgtgcacacacgccaacaggcatctgccatgctggcatctctataagggctccagtccagagaccctgggccattgaacttgctcctcaggcagaggctgagtccgcacatcacctccaggccctcagaacacctgccccagccccaccatgctcatggcgtccaccacttccgctgtgcctgggcatccctctctgcccagcctgcccagcaacagcagccaggagaggccactggacacccgggacccgctgctagcccgggcggagctggcgctgctctccatagtctttgtggctgtggccctgagcaatggcctggtgctggcggccctagctcggcggggccggcggggccactgggcacccatacacgtcttcattggccacttgtgcctggccgacctggccgtggctctgttccaagtgctgccccagctggcctggaaggccaccgaccgcttccgtgggccagatgccctgtgtcgggccgtgaagtatctgcagatggtgggcatgtatgcctcctcctacatgatcctggccatgacgctggaccgccaccgtgccatctgccgtcccatgctggcgtaccgccatggaagtggggctcactggaaccggccggtgctagtggcttgggccttctcgctccttctcagcctgccccagctcttcatcttcgcccagcgcaacgtggaaggtggcagcggggtcactgactgctgggcctgctttgcggagccctggggccgtcgcacctatgtcacctggattgccctgatggtgttcgtggcacctaccctgggtatcgccgcctgccaggtgctcatcttccgggagattcatgccagtctggtgccagggccatcagagaggcctggggggcgccgcaggggacgccggacaggcagccccggtgagggagcccacgtgtcagcagctgtggccaagactgtgaggatgacgctagtgattgtggtcgtctatgtgctgtgctgggcacccttcttcctggtgcagctgtgggccgcgtgggacccggaggcacctctggaaggggcgccctttgtgctactcatgttgctggccagcctcaacagctgcaccaacccctggatctatgcatctttcagcagcagcgtgtcctcagagctgcgaagcttgctctgctgtgcccggggacgcaccccacccagcctgggtccccaagatgagtcctgcaccaccgccagctcctccctggccaaggacacttcatcgtgaggagctgttgggtgtcttgcctctagaggctttgagaagctcagctgccttcctggggctggtcctgggagccactgggagggggacccgtggagaattggccagagcctgtggccccgaggctgggacactgtgtggccctggacaagccacagcccctgcctgggtctccacatccccagctgtatgaggagagcttcaggccccaggactgtgggggcccctcaggtcagctcactgagctgggtgtaggaggggctgcagcagaggcctgaggagtggcaggaaagagggagcaggtgcccccaggtgagacagcggtcccaggggcctgaaaaggaaggaccaggctggggccaggggaccttcctgtctccgcctttctaatccctccctcctcattctctccctaataaaaattggagctcttttccacatggcaaggggtctccttggaa

SEQ ID NO: 17 - DNA, 2776 bp, Homo sapiens
ggctcgctgcctcgcattgccacaggctcctgagaggtcgcgggcagtgcctgcggggaggcgcggggccctgctctgtagggctgaaggccgcccgaggttcgccaaggctctgggctctcgaaaggaagccaagaaaagaagctgcccaggtgaccagtcctgggagtgctctctcccaaggaagctccgagcgcccaggagcccttagccggggtctagtgccctttgaacaatctccagctcttcaaggaagtgggctgccgccgcctctcttgggacctggcctgggatcctttccccaaacgcaccccggcgatttttgcgcaccgggagccgaacccctgctgcgcgcagctggctgggctcaggcgcgcttcctcaacgtttcggagccgctgcccccagcgaagtccacattccaagctccaggggctttgagagagacgaccccaaggcaaggcgtttggagagctgctgaggagccaggggcttggaggagcgagaagacatgtattttcagctgagtctcagaaggggagaatctcctgtcaccaccagaaaagcaacagccccgaaatgtgattgcaactgactagcagagcagaggcccaggagtcactggattgatgatttagaatatgctaaaaagccagtgctttatttggggaattcaggggctttctggtgcccaagacagtgacctgcagatgaggactctgaacacctctgccatggacgggactgggctggtggtggagagggacttctctgttcgtatcctcactgcctgtttcctatcgctgctcatcctgtccacgctcctggggaacacgctggtctgtgctgccgttatcaggttccgacacctgcggtccaaggtgaccaacttctttgtcatctccttggctgtgtcagatctcttggtggcagtcctggtcatgccctggaaggcagtggctgagattgctggcttctggccctttgggtccttctgtaacatctgggtggcctttgacatcatgtgctccactgcatccatcctcaacctctgtgtgatcagcgtggacaggtattgggctatctccagccctttccggtatgagagaaagatgacccccaaggcagccttcatcctgatcagtgtggcatggaccttgtctgtactcatctccttcatcccagtgcagctcagctggcacaaggcaaaacccacaagcccctctgatggaaatgccacttccctggctgagaccatagacaactgtgactccagcctcagcaggacatatgccatctcatcctctgtaataagcttttacatccctgtggccatcatgattgtcacctacaccaggatctacaggattgctcagaaacaaatacggcgcattgcggccttggagagggcagcagtccacgccaagaattgccagaccaccacaggtaatggaaagcctgtcgaatgttctcaaccggaaagttcttttaagatgtccttcaaaagagaaactaaagtcctgaagactctgtcggtgatcatgggtgtgtttgtgtgctgttggctacctttcttcatcttgaactgcattttgcccttctgtgggtctggggagacgcagcccttctgcattgattccaacacctttgacgtgtttgtgtggtttgggtgggctaattcatccttgaaccccatcatttatgcctttaatgctgattttcggaaggcattttcaaccctcttaggatgctacagactttgccctgcgacgaataatgccatagagacggtgagtatcaataacaatggggccgcgatgttttccagccatcatgagccacgaggctccatctccaaggagtgcaatctggtttacctgatcccacatgctgtgggctcctctgaggacctgaaaaaggaggaggcagctggcatcgccagacccttggagaagctgtccccagccctatcggtcatattggactatgacactgacgtctctctggagaagatccaacccatcacacaaaacggtcagcacccaacctgaactcgcagatgaatcctgccacacatgctcatcccaaaagctagaggagattgctctggggtttgctattaagaaactaaggtacggtgagactctgaggtgtcaggagagccctctgctgctttccaacacacaattaactccgtttccaaatacattccagtgtattttctgtgttgttcatagtcaatcaaacagggacactacaaacatggggagccataagggacatgtctttggcttcagaattgtttttagaaatttattcttatcttaggatttaccaaatagggcaaagaatcaacagtgaacagcttcacttaaaatcaaatttttctgggaagaaaatgagatgggttgagtttgctgtatacaaacaggtgctaacactgttcccagcaaagttttcagattgtaaaggtaggtgcatgccttcataaattatttctaaaacattaattgaggcttacagtaggagtgagaaatttttttccagaattgagagatgttttgttgatattggttctatttatttattgtatatatggatatttttaatttatgatataataaatatatatttatcatatttaataggataaattaatgagttttatccaagaccttacaaccacatttctggccatttaactagcactttataagccaatgaagcaaacacacagactctgtgagattctaaatgttcatgtgtaacttctaga

SEQ ID NO: 18 - DNA, 2033 bp, Homo sapiens
ctcttccggacgtgacgcaagggcggggttgccggaagaagtggcgaagttacttttgagggtatttgagtagcggcggtgtgtcaggggctaaagaggaggacgaagaaaagcagagcaagggaacccagggcaacaggagtagttcactccgcgagaggccgtccacgagacccccgcgcgcagccatgagccccgccccccgctgttgcttggagaggggcgggacctggagagaggctgctccgtgaccccaccatgtcctctcctaccacgagttccctggatacccccctgcctggaaatggcccccctcagcctggcgccccttcttcttcacccactgtaaaggaggagggtccggagccgtggcccgggggtccggaccctgatgtcccaggcactgatgaggccagctcagcctgcagcacagactgggtcatcccagatcccgaagaggaaccagagcgcaagcgaaagaagggcccagccccgaagatgctgggccacgagctttgccgtgtctgtggggacaaggcctccggcttccactacaacgtgctcagctgcgaaggctgcaagggcttcttccggcgcagtgtggtccgtggtggggccaggcgctatgcctgccggggtggcggaacctgccagatggacgctttcatgcggcgcaagtgccagcagtgccggctgcgcaagtgcaaggaggcagggatgagggagcagtgcgtcctttctgaagaacagatccggaagaagaagattcggaaacagcagcaggagtcacagtcacagtcgcagtcacctgtggggccgcagggcagcagcagctcagcctctgggcctggggcttcccctggtggatctgaggcaggcagccagggctccggggaaggcgagggtgtccagctaacagcggctcaagaactaatgatccagcagttggtggcggcccaactgcagtgcaacaaacgctccttctccgaccagcccaaagtcacgccctggcccctgggcgcagacccccagtcccgagatgcccgccagcaacgctttgcccacttcacggagctggccatcatctcagtccaggagatcgtggacttcgctaagcaagtgcctggtttcctgcagctgggccgggaggaccagatcgccctcctgaaggcatccactatcgagatcatgctgctagagacagccaggcgctacaaccacgagacagagtgtatcaccttcttgaaggacttcacctacagcaaggacgacttccaccgtgcaggcctgcaggtggagttcatcaaccccatcttcgagttctcgcgggccatgcggcggctgggcctggacgacgctgagtacgccctgctcatcgccatcaacatcttctcggccgaccggcccaacgtgcaggagccgggccgcgtggaggcgttgcagcagccctacgtggaggcgctgctgtcctacacgcgcatcaagaggccgcaggaccagctgcgcttcccgcgcatgctcatgaagctggtgagcctgcgcacgctgagctctgtgcactcggagcaggtcttcgccttgcggctccaggacaagaagctgccgcctctgctgtcggagatctgggacgtccacgagtgaggggctggccacccagccccacagccttgcctgaccaccctccagcagatagacgccggcaccccttcctcttcctagggtggaaggggccctgggccgagcctgtagacctatcggctctcatcccttgggataagccccagtccaggtccaggaggctccctccctgcccagcgagtcttccagaaggggtgaaagggttgcaggtcccgaccactgacccttcccggctgccctccctccccagcttacacctcaagcccagcacgcagtgcaccttgaacagagggaggggaggacccatggctctcccccctagcccgggagaccagggccttcctcttcctctgcttttatttaataaaaactaaaaacagaaaaaaaaaaaaaaa

SEQ ID NO: 19 - DNA, 21 bp, Artificial Sequence
  PCR primer
tgccgactgcagaagtgtctt

SEQ ID NO: 20 - DNA, 22 bp, Artificial Sequence
  PCR primer
gtccctttgcttcttggacatc

SEQ ID NO: 21 - DNA, 20 bp, Artificial Sequence
  PCR primer
gggaagactttgccaaagca

SEQ ID NO: 22 - DNA, 19 bp, Artificial Sequence
  PCR primer
tcggcatcattgagttgca

SEQ ID NO: 23 - DNA, 19 bp, Artificial Sequence
  PCR primer
atcccgctccggtacaatg

SEQ ID NO: 24 - DNA, 21 bp, Artificial Sequence
  PCR primer
tccaacctagcatgggagtca

SEQ ID NO: 25 - DNA, 22 bp, Artificial Sequence
  PCR primer
attgccatcgaccgctactatg

SEQ ID NO: 26 - DNA, 19 bp, Artificial Sequence
  PCR primer
cagccgatgagcgagtgaa

SEQ ID NO: 27 - DNA, 22 bp, Artificial Sequence
  PCR primer
ccgacaagtggtacatggaaag

SEQ ID NO: 28 - DNA, 23 bp, Artificial Sequence
  PCR primer
ctccggcttgtgatgctattatg

SEQ ID NO: 29 - DNA, 23 bp, Artificial Sequence
  PCR primer
atccagggttcagctctttttct

SEQ ID NO: 30 - DNA, 20 bp, Artificial Sequence
  PCR primer
ttttccgcatgacactttcg

SEQ ID NO: 31 - DNA, 17 bp, Artificial Sequence
  PCR primer
tgcgtccgagcgtcact

SEQ ID NO: 32 - DNA, 22 bp, Artificial Sequence
  PCR primer
atgcagacatccaccacactgt

SEQ ID NO: 33 - DNA, 24 bp, Artificial Sequence
  PCR primer
tccgtgatggaccctctcatatat

SEQ ID NO: 34 - DNA, 25 bp, Artificial Sequence
  PCR primer
ggcagcaaataatctccttaaaggt

SEQ ID NO: 35 - DNA, 22 bp, Artificial Sequence
  PCR primer
ctttggtcaccatcctgatctg

SEQ ID NO: 36 - DNA, 23 bp, Artificial Sequence
  PCR primer
ttccttgttttccgtggctttat

SEQ ID NO: 37 - DNA, 23 bp, Artificial Sequence
  PCR primer
gctgcctcgggattatttagatg

SEQ ID NO: 38 - DNA, 19 bp, Artificial Sequence
  PCR primer
tctggctctacggcaggaa

SEQ ID NO: 39 - DNA, 23 bp, Artificial Sequence
  PCR primer
tgtgaggatgacgctagtgattg

SEQ ID NO: 40 - DNA, 23 bp, Artificial Sequence
  PCR primer
cagcaacatgagtagcacaaagg

SEQ ID NO: 41 - DNA, 21 bp, Artificial Sequence
  PCR primer
gtaacatctgggtggcctttg

SEQ ID NO: 42 - DNA, 20 bp, Artificial Sequence
  PCR primer
acctgtccacgctgatcaca

SEQ ID NO: 43 - DNA, 21 bp, Artificial Sequence
  PCR primer
aaagtgggcatgctgaaagaa

SEQ ID NO: 44 - DNA, 20 bp, Artificial Sequence
  PCR primer
cgcatctatcctgcgcttgt

SEQ ID NO: 45 - DNA, 21 bp, Artificial Sequence
  PCR primer
gcggatccctcttttttttcc

SEQ ID NO: 46 - DNA, 21 bp, Artificial Sequence
  PCR primer
gaggacattggagtggcagaa

SEQ ID NO: 47 - DNA, 21 bp, Artificial Sequence
  PCR primer
ttccagaccattggcttgagt

SEQ ID NO: 48 - DNA, 21 bp, Artificial Sequence
  PCR primer
actcccactgactttcctgct

SEQ ID NO: 49 - DNA, 25 bp, Artificial Sequence
  PCR primer
catgctgagtgacacagacaagaag

SEQ ID NO: 50 - DNA, 25 bp, Artificial Sequence
  PCR primer
tggtagttgttgtgagcatagtcca

SEQ ID NO: 51 - protein, 412 aa, Homo sapiens
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 52 - protein, 447 aa, Homo sapiens
MSLWLGAPVPDIPPDSAVELWKPGAQDASSQAQGGSSCILREEARMPHSAGGTAGVGLEAAEPTALLTRAEPPSEPTEIRPQKRKKGPAPKMLGNELCSVCGDKASGFHYNVLSCEGCKGFFRRSVIKGAHYICHSGGHCPMDTYMRRKCQECRLRKCRQAGMREECVLSEEQIRLKKLKRQEEEQAHATSLPPRRSSPPQILPQLSPEQLGMIEKLVAAQQQCNRRSFSDRLRVTPWPMAPDPHSREARQQRFAHFTELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLSSVHSEQVFALRLQDKKLPPLLSEIWDVHE

SEQ ID NO: 53 - protein, 500 aa, Homo sapiens [sequence omitted]

SEQ ID NO: 54 - protein, 495 aa, Homo sapiens
MSSNSDTGDLQESLKHGLTPIVSQFKMVNYSYDEDLEELCPVCGDKVSGYHYGLLTCESCKGFFKRTVQNNKRYTCIENQNCQIDKTQRKRCPYCRFQKCLSVGMKLEAVRADRMRGGRNKFGPMYKRDRALKQQKKALIRANGLKLEAMSQVIQAMPSDLTISSAIQNIHSASKGLPLNHAALPPTDYDRSPFVTSPISMTMPPHGSLQGYQTYGHFPSRAIKSEYPDPYTSSPESIMGYSYMDSYQTSSPASIPHLILELLKCEPDEPQVQAKIMAYLQQEQANRSKHEKLSTFGLMCKMADQTLFSIVEWARSSIFFRELKVDDQMKLLQNCWSELLILDHIYRQVVHGKEGSIFLVTGQQVDYSIIASQAGATLNNLMSHAQELVAKLRSLQFDQREFVCLKFLVLFSLDVKNLENFQLVEGVQEQVNAALLDYTMCNYPQQTEKFGQLLLRLPEIRAISMQAEEYLYYKHLNGDVPYNNLLIEMLHAKRA

SEQ ID NO: 55 - protein, 541 aa, Homo sapiens
MSSNSDTGDLQESLKHGLTPIGAGLPDRHGSPIPARGRLVMLPKVETEALGLARSHGEQGQMPENMQVSQFKMVNYSYDEDLEELCPVCGDKVSGYHYGLLTCESCKGFFKRTVQNNKRYTCIENQNCQIDKTQRKRCPYCRFQKCLSVGMKLEAVRADRMRGGRNKFGPMYKRDRALKQQKKALIRANGLKLEAMSQVIQAMPSDLTISSAIQNIHSASKGLPLNHAALPPTDYDRSPFVTSPISMTMPPHGSLQGYQTYGHFPSRAIKSEYPDPYTSSPESIMGYSYMDSYQTSSPASIPHLILELLKCEPDEPQVQAKIMAYLQQEQANRSKHEKLSTFGLMCKMADQTLFSIVEWARSSIFFRELKVDDQMKLLQNCWSELLILDHIYRQVVHGKEGSIFLVTGQQVDYSIIASQAGATLNNLMSHAQELVAKLRSLQFDQREFVCLKFLVLFSLDVKNLENFQLVEGVQEQVNAALLDYTMCNYPQQTEKFGQLLLRLPEIRAISMQAEEYLYYKHLNGDVPYNNLLIEMLHAKRA

SEQ ID NO: 56 - protein, 361 aa, Homo sapiens
MNESRWTEWRILNMSSGIVNASERHSCPLGFGHYSVVDVCIFETVVIVLLTFLIIAGNLTVIFAFHCAPLLHHYTTSYFIQTMAYADLFVGVSCLVPTLSLLHYSTGVHESLTCRVFGYIISVLKSVSMACLACISVDRYLAITKPLSYNQLVTPCRLRICIILIWIYSCLIFLPSFFGWGKPGYHGDIFEWCATSWLTSAYFTGFIVCLLYAPAAFVVCFTYFHIFKICRQHTKEINDRRARFPSHEVDSSRETGHSPDRRYAMVLFRITSVFYMLWLPYIIYFLLESSRVLDNPTLSFLTTWLAVSNSFCNCVIYSLSNGVFRLGLRRLFETMCTSCMCVKDQEAQEPKPRKRANSCSI

SEQ ID NO: 57 - protein, 407 aa, Homo sapiens
MSLNSSLSCRKELSNLTEEEGGEGGVIITQFIAIIVITIFVCLGNLVIVVTLYKKSYLLTLSNKFVFSLTLSNFLLSVLVLPFVVTSSIRREWIFGVVWCNFSALLYLLISSASMLTLGVIAIDRYYAVLYPMVYPMKITGNRAVMALVYIWLHSLIGCLPPLFGWSSVEFDEFKWMCVAAWHREPGYTAFWQIWCALFPFLVMLVCYGFIFRVARVKARKVHCGTVVIVEEDAQRTGRKNSSTSTSSSGSRRNAFQGVVYSANQCKALITILVVLGAFMVTWGPYMVVIASEALWGKSSVSPSLETWATWLSFASAVCHPLIYGLWNKTVRKELLGMCFGDRYYREPFVQRQRTSRLFSISNRITDLGLSPHLTALMAGGQPLGHSSSTGDTGFSCSQDSGNLRAL

SEQ ID NO: 58 - protein, 529 aa, Homo sapiens
MSLNSSLSCRKELSNLTEEEGGEGGVIITQFIAIIVITIFVCLGNLVIVVTLYKKSYLLTLSNKFVFSLTLSNFLLSVLVLPFVVTSSIRREWIFGVVWCNFSALLYLLISSASMLTLGVIAIDRYYAVLYPMVYPMKITGNRAVMALVYIWLHSLIGCLPPLFGWSSVEFDEFKWMCVAAWHREPGYTAFWQIWCALFPFLVMLVCYGFIFRVARVKARKVHCGTVVIVEEDAQRTGRKNSSTSTSSSGSRRNAFQGVVYSANQCKALITILVVLGAFMVTWGPYMVVIASEALWGKSSVSPSLETWATWLSFASAVCHPLIYGLWNKTVRKELLGMCFGDRYYREPFVQRQRTSRLFSISNRITDLGLSPHLTALMAGGQPLGHSSSTGDTGFSCSQDSGTDMMLLEDYTSDDNPPSHCTCPPKRRSSVTFEDEVEQIKEAAKNSILHVKAEVHKSLDSYAASLAKAIEAEAKINLFGEEALPGVLVTARTVPGGGFGGRRGSRTLVSQRLQLQSIEEGDVLAAEQR

SEQ ID NO: 59 - protein, 546 aa, Homo sapiens
MKVVQHALPTPRRGALTMSLNSSLSCRKELSNLTEEEGGEGGVIITQFIAIIVITIFVCLGNLVIVVTLYKKSYLLTLSNKFVFSLTLSNFLLSVLVLPFVVTSSIRREWIFGVVWCNFSALLYLLISSASMLTLGVIAIDRYYAVLYPMVYPMKITGNRAVMALVYIWLHSLIGCLPPLFGWSSVEFDEFKWMCVAAWHREPGYTAFWQIWCALFPFLVMLVCYGFIFRVARVKARKGHCGTVVIVEEDAQRTGRKNSSTSTSSSGSRRNAFQGVVYSANQCKALITILVVLGAFMVTWGPYMVVIASEALWGKSSVSPSLETWATWLSFASAVCHPLIYGLWNKTVRKELLGMCFGDRYYREPFVQRQRTSRLFSISNRITDLGLSPHLTALMAGGQPLGHSSSTGDTGFSCSQDSGTDMMLLEDYTSDDNPPSHCTCPPKRRSSVTFEDEVEQIKEAAKNSILHVKAEVHKSLDSYAASLAKAIEAEAKINLFGEEALPGVLVTARTVPGGGFGGRRGSRTLVSQRLQLQSIEEGDVLAAEQR

SEQ ID NO: 60 - protein, 337 aa, Homo sapiens
MNSTCIEEQHDLDHYLFPIVYIFVIIVSIPANIGSLCVSFLQAKKESELGIYLFSLSLSDLLYALTLPLWIDYTWNKDNWTFSPALCKGSAFLMYMNFYSSTAFLTCIAVDRYLAVVYPLKFFFLRTRRFALMVSLSIWILETIFNAVMLWEDETVVEYCDAEKSNFTLCYDKYPLEKWQINLNLFRTCTGYAIPLVTILICNRKVYQAVRHNKATENKEKKRIIKLLVSITVTFVLCFTPFHVMLLIRCILEHAVNFEDHSNSGKRTYTMYRITVALTSLNCVADPILYCFVTETGRYDMWNILKFCTGRCNTSQRQRKRILSVSTKDTMELEVLE

SEQ ID NO: 61 - protein, 458 aa, Homo sapiens
MDSVELCLPESFSLHYEEELLCRMSNKDRHIDSSCSSFIKTEPSSPASLTDSVNHHSPGGSSDASGSYSSTMNGHQNGLDSPPLYPSAPILGGSGPVRKLYDDCSSTIVEDPQTKCEYMLNSMPKRLCLVCGDIASGYHYGVASCEACKAFFKRTIQGNIEYSCPATNECEITKRRRKSCQACRFMKCLKVGMLKEGVRLDRVRGGRQKYKRRIDAENSPYLNPQLVQPAKKPYNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTSTKAVQHFYNIKLEGKVPMHKLFLEMLEAKV

SEQ ID NO: 62 - protein, 435 aa, Homo sapiens
MSNKDRHIDSSCSSFIKTEPSSPASLTDSVNHHSPGGSSDASGSYSSTMNGHQNGLDSPPLYPSAPILGGSGPVRKLYDDCSSTIVEDPQTKCEYMLNSMPKRLCLVCGDIASGYHYGVASCEACKAFFKRTIQGNIEYSCPATNECEITKRRRKSCQACRFMKCLKVGMLKEGVRLDRVRGGRQKYKRRIDAENSPYLNPQLVQPAKKPYNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTSTKAVQHFYNIKLEGKVPMHKLFLEMLEAKV

SEQ ID NO: 63 - protein, 435 aa, Homo sapiens; identical to SEQ ID NO: 62

SEQ ID NO: 64 - protein, 334 aa, Homo sapiens
MNEDLKVNLSGLPRDYLDAAAAENISAAVSSRVPAVEPEPELVVNPWDIVLCTSGTLISCENAIVVLIIFHNPSLRAPMFLLIGSLALADLLAGIGLITNFVFAYLLQSEATKLVTIGLIVASFSASVCSLLAITVDRYLSLYYALTYHSERTVTFTYVMLVMLWGTSICLGLLPVMGWNCLRDESTCSVVRPLTKNNAAILSVSFLFMFALMLQLYIQICKIVMRHAHQIALQHHFLATSHYVTTRKGVSTLAIILGTFAACWMPFTLYSLIADYTYPSIYTYATLLPATYNSIINPVIYAFRNQEIQKALCLICCGCIPSSLAQRARSPSDV

SEQ ID NO: 65 - protein, 325 aa, Homo sapiens
MNSSFHLHFLDLNLNATEGNLSGPNVKNKSSPCEDMGIAVEVFLTLGVISLLENILVIGAIVKNKNLHSPMYFFVCSLAVADMLVSMSSAWETITIYLLNNKHLVIADAFVRHIDNVFDSMICISVVASMCSLLAIAVDRYVTIFYALRYHHIMTARRSGAIIAGIWAFCTGCGIVFILYSESTYVILCLISMFFAMLFLLVSLYIHMFLLARTHVKRIAALPGASSARQRTSMQGAVTVTMLLGVFTVCWAPFFLHLTLMLSCPQNLYCSRFMSHFNMYLILIMCNSVMDPLIYAFRSQEMRKTFKEIICCRGFRIACSFPRRD

SEQ ID NO: 66 - protein, 371 aa, Homo sapiens
MLMASTTSAVPGHPSLPSLPSNSSQERPLDTRDPLLARAELALLSIVFVAVALSNGLVLAALARRGRRGHWAPIHVFIGHLCLADLAVALFQVLPQLAWKATDRFRGPDALCRAVKYLQMVGMYASSYMILAMTLDRHRAICRPMLAYRHGSGAHWNRPVLVAWAFSLLLSLPQLFIFAQRNVEGGSGVTDCWACFAEPWGRRTYVTWIALMVFVAPTLGIAACQVLIFREIHASLVPGPSERPGGRRRGRRTGSPGEGAHVSAAVAKTVRMTLVIVVVYVLCWAPFFLVQLWAAWDPEAPLEGAPFVLLMLLASLNSCTNPWIYASFSSSVSSELRSLLCCARGRTPPSLGPQDESCTTASSSLAKDTSS

SEQ ID NO: 67 - protein, 446 aa, Homo sapiens
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 68 - protein, 460 aa, Homo sapiens
MSSPTTSSLDTPLPGNGPPQPGAPSSSPTVKEEGPEPWPGGPDPDVPGTDEASSACSTDWVIPDPEEEPERKRKKGPAPKMLGHELCRVCGDKASGFHYNVLSCEGCKGFFRRSVVRGGARRYACRGGGTCQMDAFMRRKCQQCRLRKCKEAGMREQCVLSEEQIRKKKIRKQQQESQSQSQSPVGPQGSSSSASGPGASPGGSEAGSQGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE

SEQ ID NO: 69 - protein, 9 aa, Artificial Sequence
  Protein domain fragment
MPIMGSSVY

SEQ ID NO: 70 - protein, 23 aa, Artificial Sequence
  Protein domain fragment
ITVELAIAVLAILGNVLVCWAVW

SEQ ID NO: 71 - protein, 11 aa, Artificial Sequence
  Protein domain fragment
LNSNLQNVTNY

SEQ ID NO: 72 - protein, 23 aa, Artificial Sequence
  Protein domain fragment
FVVSLAAADIAVGVLAIPFAITI

SEQ ID NO: 73 - protein, 9 aa, Artificial Sequence
  Protein domain fragment
STGFCAACH

SEQ ID NO: 74 - protein, 23 aa, Artificial Sequence
  Protein domain fragment
GCLFIACFVLVLTQSSIFSLLAI

SEQ ID NO: 75 - protein, 22 aa, Artificial Sequence
  Protein domain fragment
AIDRYIAIRIPLRYNGLVTGTR

SEQ ID NO: 76 - protein, 23 aa, Artificial Sequence
  Protein domain fragment
AKGIIAICWVLSFAIGLTPMLGW

SEQ ID NO: 77 - protein, 35 aa, Artificial Sequence
  Protein domain fragment
NNCGQPKEGKNHSQGCGEGQVACLFEDVVPMNYMV

SEQ ID NO: 78 - protein, 23 aa, Artificial Sequence
  Protein domain fragment
YFNFFACVLVPLLLMLGVYLRIF

SEQ ID NO: 79 - protein, 28 aa, Artificial Sequence
  Protein domain fragment
LAARRQLKQMESQPLPGERARSTLQKEV

SEQ ID NO: 80 - protein, 23 aa, Artificial Sequence
  Protein domain fragment
HAAKSLAIIVGLFALCWLPLHII

SEQ ID NO: 81 - protein, 14 aa, Artificial Sequence
  Protein domain fragment
NCFTFFCPDCSHAP

SEQ ID NO: 82 - protein, 23 aa, Artificial Sequence
  Protein domain fragment
LWLMYLAIVLSHTNSVVNPFIYA

SEQ ID NO: 83 - protein, 123 aa, Artificial Sequence
  Protein domain fragment
YRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 84 - protein, 43 aa, Artificial Sequence
  Protein domain fragment
MNESRWTEWRILNMSSGIVNASERHSCPLGFGHYSVVDVCIFE

SEQ ID NO: 85 - protein, 23 aa, Artificial Sequence
  Protein domain fragment
TVVIVLLTFLIIAGNLTVIFAFH

SEQ ID NO: 86 - protein, 11 aa, Artificial Sequence
  Protein domain fragment
CAPLLHHYTTS

SEQ ID NO: 87 - protein, 23 aa, Artificial Sequence
  Protein domain fragment
YFIQTMAYADLFVGVSCLVPTLS

SEQ ID NO: 88 - protein, 14 aa, Artificial Sequence
  Protein domain fragment
LLHYSTGVHESLTC

SEQ ID NO: 89 - protein, 23 aa, Artificial Sequence
  Protein domain fragment
RVFGYIISVLKSVSMACLACISV

SEQ ID NO: 90 - protein, 20 aa, Artificial Sequence
  Protein domain fragment
DRYLAITKPLSYNQLVTPCR

SEQ ID NO: 91 - protein, 23 aa, Artificial Sequence
  Protein domain fragment
LRICIILIWIYSCLIFLPSFFGW

SEQ ID NO: 92 - protein, 21 aa, Artificial Sequence
  Protein domain fragment
GKPGYHGDIFEWCATSWLTSA

SEQ ID NO: 93 - protein, 23 aa, Artificial Sequence
  Protein domain fragment
YFTGFIVCLLYAPAAFVVCFTYF

SEQ ID NO: 94 - protein, 40 aa, Artificial Sequence
  Protein domain fragment
HIFKICRQHTKEINDRRARFPSHEVDSSRETGHSPDRRYA

SEQ ID NO: 95 - protein, 23 aa, Artificial Sequence
  Protein domain fragment
MVLFRITSVFYMLWLPYIIYFLL

SEQ ID NO: 96 - protein, 14 aa, Artificial Sequence
  Protein domain fragment
ESSRVLDNPTLSFL

SEQ ID NO: 97 - protein, 23 aa, Artificial Sequence
  Protein domain fragment
TTWLAVSNSFCNCVIYSLSNGVF

SEQ ID NO: 98 - protein, 37 aa, Artificial Sequence
  Protein domain fragment
RLGLRRLFETMCTSCMCVKDQEAQEPKPRKRANSCSI

SEQ ID NO: 99 - protein, 30 aa, Artificial Sequence
  Protein domain fragment
MSLNSSLSCRKELSNLTEEEGGEGGVIITQ

SEQ ID NO: 100 - protein, 23 aa, Artificial Sequence
  Protein domain fragment
FIAIIVITIFVCLGNLVIVVTLY

SEQ ID NO: 101 - protein, 11 aa, Artificial Sequence
  Protein domain fragment
KKSYLLTLSNK

SEQ ID NO: 102 - protein, 23 aa, Artificial Sequence
  Protein domain fragment
FVFSLTLSNFLLSVLVLPFVVTS

SEQ ID NO: 103 - protein, 14 aa, Artificial Sequence
  Protein domain fragment
SIRREWIFGVVWCN

SEQ ID NO: 104 - protein, 22 aa, Artificial Sequence
  Protein domain fragment
FSALLYLLISSASMLTLGVIAI

SEQ ID NO: 105 - protein, 20 aa, Artificial Sequence
  Protein domain fragment
DRYYAVLYPMVYPMKITGNR

SEQ ID NO: 106 - protein, 23 aa, Artificial Sequence
  Protein domain fragment
AVMALVYIWLHSLIGCLPPLFGW

SEQ ID NO: 107 - protein, 23 aa, Artificial Sequence
  Protein domain fragment
SSVEFDEFKWMCVAAWHREPGYT

SEQ ID NO: 108 - protein, 23 aa, Artificial Sequence
  Protein domain fragment
AFWQIWCALFPFLVMLVCYGFIF

SEQ ID NO: 109 - protein, 55 aa, Artificial Sequence
  Protein domain fragment
RVARVKARKVHCGTVVIVEEDAQRTGRKNSSTSTSSSGSRRNAFQGVVYSANQCK

SEQ ID NO: 110 - protein, 23 aa, Artificial Sequence
  Protein domain fragment
ALITILVVLGAFMVTWGPYMVVI

SEQ ID NO: 111 - protein, 14 aa, Artificial Sequence
  Protein domain fragment
ASEALWGKSSVSPS

SEQ ID NO: 112 - protein, 23 aa, Artificial Sequence
  Protein domain fragment
LETWATWLSFASAVCHPLIYGLW

SEQ ID NO: 113 - protein, 80 aa, Artificial Sequence
  Protein domain fragment
NKTVRKELLGMCFGDRYYREPFVQRQRTSRLFSISNRITDLGLSPHLTALMAGGQPLGHSSSTGDTGFSCSQDSGNLRAL

SEQ ID NO: 114 - protein, 30 aa, Artificial Sequence; identical to SEQ ID NO: 99
  Protein domain fragment

SEQ ID NO: 115 - protein, 23 aa, Artificial Sequence
  Protein domain fragment
FIAIIVITIFVCLGNLVIVVTLY

SEQ ID NO: 116 - protein, 11 aa, Artificial Sequence
  Protein domain fragment
KKSYLLTLSNK

SEQ ID NO: 117 - protein, 23 aa, Artificial Sequence
  Protein domain fragment
FVFSLTLSNFLLSVLVLPFVVTS

SEQ ID NO: 118 - protein, 14 aa, Artificial Sequence
  Protein domain fragment
SIRREWIFGVVWCN

SEQ ID NO: 119 - protein, 22 aa, Artificial Sequence
  Protein domain fragment
FSALLYLLISSASMLTLGVIAI

SEQ ID NO: 120 - protein, 20 aa, Artificial Sequence
  Protein domain fragment
DRYYAVLYPMVYPMKITGNR

SEQ ID NO: 121 - protein, 23 aa, Artificial Sequence
  Protein domain fragment
AVMALVYIWLHSLIGCLPPLFGW

SEQ ID NO: 122 - protein, 23 aa, Artificial Sequence
  Protein domain fragment
SSVEFDEFKWMCVAAWHREPGYT

SEQ ID NO: 123 - protein, 23 aa, Artificial Sequence
  Protein domain fragment
AFWQIWCALFPFLVMLVCYGFIF

SEQ ID NO: 124 - protein, 55 aa, Artificial Sequence; identical to SEQ ID NO: 109
  Protein domain fragment

SEQ ID NO: 125 - protein, 23 aa, Artificial Sequence
  Protein domain fragment
ALITILVVLGAFMVTWGPYMVVI

SEQ ID NO: 126 - protein, 14 aa, Artificial Sequence
  Protein domain fragment
ASEALWGKSSVSPS

SEQ ID NO: 127 - protein, 23 aa, Artificial Sequence
  Protein domain fragment
LETWATWLSFASAVCHPLIYGLW

SEQ ID NO: 128 - protein, 202 aa, Artificial Sequence
  Protein domain fragment
NKTVRKELLGMCFGDRYYREPFVQRQRTSRLFSISNRITDLGLSPHLTALMAGGQPLGHSSSTGDTGFSCSQDSGTDMMLLEDYTSDDNPPSHCTCPPKRRSSVTFEDEVEQIKEAAKNSILHVKAEVHKSLDSYAASLAKAIEAEAKINLFGEEALPGVLVTARTVPGGGFGGRRGSRTLVSQRLQLQSIEEGDVLAAEQR

SEQ ID NO: 129 - protein, 47 aa, Artificial Sequence
  Protein domain fragment
MKVVQHALPTPRRGALTMSLNSSLSCRKELSNLTEEEGGEGGVIITQ

SEQ ID NO: 130 - protein, 23 aa, Artificial Sequence
  Protein domain fragment
FIAIIVITIFVCLGNLVIVVTLY

SEQ ID NO: 131 - protein, 11 aa, Artificial Sequence
  Protein domain fragment
KKSYLLTLSNK

SEQ ID NO: 132 - protein, 23 aa, Artificial Sequence
  Protein domain fragment
FVFSLTLSNFLLSVLVLPFVVTS

SEQ ID NO: 133 - protein, 14 aa, Artificial Sequence
  Protein domain fragment
SIRREWIFGVVWCN

SEQ ID NO: 134 - protein, 22 aa, Artificial Sequence
  Protein domain fragment
FSALLYLLISSASMLTLGVIAI

SEQ ID NO: 135 - protein, 20 aa, Artificial Sequence
  Protein domain fragment
DRYYAVLYPMVYPMKITGNR

SEQ ID NO: 136 - protein, 23 aa, Artificial Sequence
  Protein domain fragment
AVMALVYIWLHSLIGCLPPLFGW

SEQ ID NO: 137 - protein, 23 aa, Artificial Sequence
  Protein domain fragment
SSVEFDEFKWMCVAAWHREPGYT

SEQ ID NO: 138 - protein, 23 aa, Artificial Sequence
  Protein domain fragment
AFWQIWCALFPFLVMLVCYGFIF

SEQ ID NO: 139 - protein, 55 aa, Artificial Sequence
  Protein domain fragment
RVARVKARKGHCGTVVIVEEDAQRTGRKNSSTSTSSSGSRRNAFQGVVYSANQCK

SEQ ID NO: 140 - protein, 23 aa, Artificial Sequence
  Protein domain fragment
ALITILVVLGAFMVTWGPYMVVI

SEQ ID NO: 141 - protein, 14 aa, Artificial Sequence
  Protein domain fragment
ASEALWGKSSVSPS

SEQ ID NO: 142 - protein, 23 aa, Artificial Sequence
  Protein domain fragment
LETWATWLSFASAVCHPLIYGLW

SEQ ID NO: 143 - protein, 202 aa, Artificial Sequence; identical to SEQ ID NO: 128
  Protein domain fragment

SEQ ID NO: 144 - protein, 14 aa, Artificial Sequence
  Protein domain fragment
MNSTCIEEQHDLDH

SEQ ID NO: 145 - protein, 23 aa, Artificial Sequence
  Protein domain fragment
YLFPIVYIFVIIVSIPANIGSLC

SEQ ID NO: 146 - protein, 11 aa, Artificial Sequence
  Protein domain fragment
VSFLQAKKESE

SEQ ID NO: 147 - protein, 23 aa, Artificial Sequence
  Protein domain fragment
LGIYLFSLSLSDLLYALTLPLWI

SEQ ID NO: 148 - protein, 19 aa, Artificial Sequence
  Protein domain fragment
DYTWNKDNWTFSPALCKGS

SEQ ID NO: 149 - protein, 20 aa, Artificial Sequence
  Protein domain fragment
AFLMYMNFYSSTAFLTCIAV

SEQ ID NO: 150 - protein, 19 aa, Artificial Sequence
  Protein domain fragment
DRYLAVVYPLKFFFLRTRR

SEQ ID NO: 151 - protein, 23 aa, Artificial Sequence
  Protein domain fragment
FALMVSLSIWILETIFNAVMLWE

SEQ ID NO: 152 - protein, 28 aa, Artificial Sequence
  Protein domain fragment
DETVVEYCDAEKSNFTLCYDKYPLEKWQ

SEQ ID NO: 153 - protein, 23 aa, Artificial Sequence
  Protein domain fragment
INLNLFRTCTGYAIPLVTILICN

SEQ ID NO: 154 - protein, 20 aa, Artificial Sequence
  Protein domain fragment
RKVYQAVRHNKATENKEKKR

SEQ ID NO: 155 - protein, 23 aa, Artificial Sequence
  Protein domain fragment
IIKLLVSITVTFVLCFTPFHVML

SEQ ID NO: 156 - protein, 24 aa, Artificial Sequence
  Protein domain fragment
LIRCILEHAVNFEDHSNSGKRTYT

SEQ ID NO: 157 - protein, 23 aa, Artificial Sequence
  Protein domain fragment
MYRITVALTSLNCVADPILYCFV

SEQ ID NO: 158 - protein, 44 aa, Artificial Sequence
  Protein domain fragment
TETGRYDMWNILKFCTGRCNTSQRQRKRILSVSTKDTMELEVLE

SEQ ID NO: 159 - protein, 46 aa, Artificial Sequence
  Protein domain fragment
MNEDLKVNLSGLPRDYLDAAAAENISAAVSSRVPAVEPEPELVVNP

SEQ ID NO: 160 - protein, 23 aa, Artificial Sequence
  Protein domain fragment
WDIVLCTSGTLISCENAIVVLII

SEQ ID NO: 161 - protein, 12 aa, Artificial Sequence
  Protein domain fragment
FHNPSLRAPMFL

SEQ ID NO: 162 - protein, 23 aa, Artificial Sequence
  Protein domain fragment
LIGSLALADLLAGIGLITNFVFA

SEQ ID NO: 163 - protein, 9 aa, Artificial Sequence
  Protein domain fragment
YLLQSEATK

SEQ ID NO: 164 - protein, 23 aa, Artificial Sequence
  Protein domain fragment
LVTIGLIVASFSASVCSLLAITV

SEQ ID NO: 165 - protein, 20 aa, Artificial Sequence
  Protein domain fragment
DRYLSLYYALTYHSERTVTF

SEQ ID NO: 166 - protein, 23 aa, Artificial Sequence
  Protein domain fragment
TYVMLVMLWGTSICLGLLPVMGW

SEQ ID NO: 167 - protein, 19 aa, Artificial Sequence
  Protein domain fragment
NCLRDESTCSVVRPLTKNN

SEQ ID NO: 168 - protein, 23 aa, Artificial Sequence
  Protein domain fragment
AAILSVSFLFMFALMLQLYIQIC

SEQ ID NO: 169 - protein, 27 aa, Artificial Sequence
  Protein domain fragment
KIVMRHAHQIALQHHFLATSHYVTTRK

SEQ ID NO: 170 - protein, 23 aa, Artificial Sequence
  Protein domain fragment
GVSTLAIILGTFAACWMPFTLYS

SEQ ID NO: 171 - protein, 9 aa, Artificial Sequence
  Protein domain fragment
LIADYTYPS

SEQ ID NO: 172 - protein, 23 aa, Artificial Sequence
  Protein domain fragment
IYTYATLLPATYNSIINPVIYAF

SEQ ID NO: 173 - protein, 31 aa, Artificial Sequence
  Protein domain fragment
RNQEIQKALCLICCGCIPSSLAQRARSPSDV

SEQ ID NO: 174 - protein, 36 aa, Artificial Sequence
  Protein domain fragment
MNSSFHLHFLDLNLNATEGNLSGPNVKNKSSPCEDM

SEQ ID NO: 175 - protein, 23 aa, Artificial Sequence
  Protein domain fragment
GIAVEVFLTLGVISLLENILVIG

SEQ ID NO: 176 - protein, 12 aa, Artificial Sequence
  Protein domain fragment
AIVKNKNLHSPM

SEQ ID NO: 177 - protein, 23 aa, Artificial Sequence
  Protein domain fragment
YFFVCSLAVADMLVSMSSAWETI

SEQ ID NO: 178 - protein, 21 aa, Artificial Sequence
  Protein domain fragment
TIYLLNNKHLVIADAFVRHID

SEQ ID NO: 179 - protein, 23 aa, Artificial Sequence
  Protein domain fragment
NVFDSMICISVVASMCSLLAIAV

SEQ ID NO: 180 - protein, 20 aa, Artificial Sequence
  Protein domain fragment
DRYVTIFYALRYHHIMTARR

SEQ ID NO: 181 - protein, 23 aa, Artificial Sequence
  Protein domain fragment
SGAIIAGIWAFCTGCGIVFILYS

SEQ ID NO: 182 - protein, 3 aa, Artificial Sequence
  Protein domain fragment
EST

SEQ ID NO: 183 - protein, 23 aa, Artificial Sequence
  Protein domain fragment
YVILCLISMFFAMLFLLVSLYIH

SEQ ID NO: 184 - protein, 30 aa, Artificial Sequence
  Protein domain fragment
MFLLARTHVKRIAALPGASSARQRTSMQGA

SEQ ID NO: 185 - protein, 23 aa, Artificial Sequence
  Protein domain fragment
VTVTMLLGVFTVCWAPFFLHLTL

SEQ ID NO: 186 - protein, 14 aa, Artificial Sequence
  Protein domain fragment
MLSCPQNLYCSRFM

SEQ ID NO: 187 - protein, 23 aa, Artificial Sequence
  Protein domain fragment
SHFNMYLILIMCNSVMDPLIYAF

SEQ ID NO: 188 - protein, 28 aa, Artificial Sequence
  Protein domain fragment
RSQEMRKTFKEIICCRGFRIACSFPRRD

SEQ ID NO: 189 - protein, 40 aa, Artificial Sequence
  Protein domain fragment
MLMASTTSAVPGHPSLPSLPSNSSQERPLDTRDPLLARAE

SEQ ID NO: 190 - protein, 23 aa, Artificial Sequence
  Protein domain fragment
LALLSIVFVAVALSNGLVLAALA

SEQ ID NO: 191 - protein, 12 aa, Artificial Sequence
  Protein domain fragment
RRGRRGHWAPIH

SEQ ID NO: 192 - protein, 23 aa, Artificial Sequence
  Protein domain fragment
VFIGHLCLADLAVALFQVLPQLA

SEQ ID NO: 193 - protein, 19 aa, Artificial Sequence
  Protein domain fragment
WKATDRFRGPDALCRAVKY

SEQ ID NO: 194 - protein, 18 aa, Artificial Sequence
  Protein domain fragment
LQMVGMYASSYMILAMTL

SEQ ID NO: 195 - protein, 20 aa, Artificial Sequence
  Protein domain fragment
DRHRAICRPMLAYRHGSGAH

SEQ ID NO: 196 - protein, 23 aa, Artificial Sequence
  Protein domain fragment
WNRPVLVAWAFSLLLSLPQLFIF

SEQ ID NO: 197 - protein, 27 aa, Artificial Sequence
  Protein domain fragment
AQRNVEGGSGVTDCWACFAEPWGRRTY

SEQ ID NO: 198 - protein, 23 aa, Artificial Sequence
  Protein domain fragment
VTWIALMVFVAPTLGIAACQVLI

SEQ ID NO: 199 - protein, 45 aa, Artificial Sequence
  Protein domain fragment
FREIHASLVPGPSERPGGRRRGRRTGSPGEGAHVSAAVAKTVRMT

SEQ ID NO: 200 - protein, 23 aa, Artificial Sequence
  Protein domain fragment
LVIVVVYVLCWAPFFLVQLWAAW

SEQ ID NO: 201 - protein, 9 aa, Artificial Sequence
  Protein domain fragment
DPEAPLEGA

SEQ ID NO: 202 - protein, 23 aa, Artificial Sequence
  Protein domain fragment
PFVLLMLLASLNSCTNPWIYASF

SEQ ID NO: 203 - protein, 43 aa, Artificial Sequence
  Protein domain fragment
SSSVSSELRSLLCCARGRTPPSLGPQDESCTTASSSLAKDTSS

SEQ ID NO: 204 - protein, 28 aa, Artificial Sequence
  Protein domain fragment
MRTLNTSAMDGTGLVVERDFSVRILTAC

SEQ ID NO: 205 - protein, 23 aa, Artificial Sequence
  Protein domain fragment
FLSLLILSTLLGNTLVCAAVIRF

SEQ ID NO: 206 - protein, 6 aa, Artificial Sequence
  Protein domain fragment
RHLRSK

SEQ ID NO: 207 - protein, 23 aa, Artificial Sequence
  Protein domain fragment
VTNFFVISLAVSDLLVAVLVMPW

SEQ ID NO: 208 - protein, 14 aa, Artificial Sequence
  Protein domain fragment
KAVAEIAGFWPFGS

SEQ ID NO: 209 - protein, 23 aa, Artificial Sequence
  Protein domain fragment
FCNIWVAFDIMCSTASILNLCVI

SEQ ID NO: 210 - protein, 21 aa, Artificial Sequence
  Protein domain fragment
SVDRYWAISSPFRYERKMTPK

SEQ ID NO: 211 - protein, 23 aa, Artificial Sequence
  Protein domain fragment
AAFILISVAWTLSVLISFIPVQL

SEQ ID NO: 212 - protein, 34 aa, Artificial Sequence
  Protein domain fragment
SWHKAKPTSPSDGNATSLAETIDNCDSSLSRTYA

SEQ ID NO: 213 - protein, 23 aa, Artificial Sequence
  Protein domain fragment
ISSSVISFYIPVAIMIVTYTRIY

SEQ ID NO: 214 - protein, 54 aa, Artificial Sequence
  Protein domain fragment
RIAQKQIRRIAALERAAVHAKNCQTTTGNGKPVECSQPESSFKMSFKRETKVLK

SEQ ID NO: 215 - protein, 23 aa, Artificial Sequence
  Protein domain fragment
TLSVIMGVFVCCWLPFFILNCIL

SEQ ID NO: 216 - protein, 19 aa, Artificial Sequence
  Protein domain fragment
PFCGSGETQPFCIDSNTFD

SEQ ID NO: 217 - protein, 20 aa, Artificial Sequence
  Protein domain fragment
VFVWFGWANSSLNPIIYAFN

SEQ ID NO: 218 - protein, 112 aa, Artificial Sequence
  Protein domain fragment
ADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 219 - protein, 286 aa, Artificial Sequence
  Protein domain fragment
GMREECVLSEEQIRLKKLKRQEEEQAHATSLPPRASSPPQILPQLSPEQLGMIEKLVAAQQQCNRRSFSDRLRVTPWPMAPDPHSREARQQRFAHFTELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLSSVHSEQVFALRLQDKKLPPLLSEIWDVHE

SEQ ID NO: 220 - protein, 306 aa, Artificial Sequence
  Protein domain fragment
MDTYMRRKCQECRLRKCRQAGMREECVLSEEQIRLKKLKRQEEEQAHATSLPPRRSSPPQILPQLSPEQLGMIEKLVAAQQQCNRRSFSDRLRVTPWPMAPDPHSREARQQRFAHFTELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLSSVHSEQVFALRLQDKKLPPLLSEIWDVHE

SEQ ID NO: 221 - protein, 233 aa, Artificial Sequence
  Protein domain fragment
EKLVAAQQQCNRRSFSDRLRVTPWPMAPDPHSREARQQRFAHFTELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLSSVHSEQVFALRLQDKKLPPLLSEIWDVHE

SEQ ID NO: 222 - protein, 160 aa, Artificial Sequence
  Protein domain fragment
ELAIVSVQEIVDFAKQLPGFLQLSREDQIALLKTSAIEVMLLETSRRYNPGSESITFLKDFSYNREDFAKAGLQVEFINPIFEFSRAMNELQLNDAEFALLIAISIFSADRPNVQDQLQVERLQHTYVEALHAYVSIHHPHDRLMFPRMLMKLVSLRTLS

SEQ ID NO: 223 - protein, 355 aa, Artificial Sequence
  Protein domain fragment
RANGLKLEAMSQVIQAMPSDLTISSAIQNIHSASKGLPLNHAALPPTDYDRSPFVTSPISMTMPPHGSLQGYQTYGHFPSRAIKSEYPDPYTSSPESIMGYSYMDSYQTSSPASIPHLILELLKCEPDEPQVQAKIMAYLQQEQANRSKHEKLSTFGLMCKMADQTLFSIVEWARSSIFFRELKVDDQMKLLQNCWSELLILDHIYRQVVHGKEGSIFLVTGQQVDYSIIASQAGATLNNLMSHAQELVAKLRSLQFDQREFVCLKFLVLFSLDVKNLENFQLVEGVQEQVNAALLDYTMCNYPQQTEKFGQLLLRLPEIRAISMQAEEYLYYKHLNGDVPYNNLLIEMLHAKRA

SEQ ID NO: 224 - protein, 310 aa, Artificial Sequence
  Protein domain fragment
PTDYDRSPFVTSPISMTMPPHGSLQGYQTYGHFPSRAIKSEYPDPYTSSPESIMGYSYMDSYQTSSPASIPHLILELLKCEPDEPQVQAKIMAYLQQEQANRSKHEKLSTFGLMCKMADQTLFSIVEWARSSIFFRELKVDDQMKLLQNCWSELLILDHIYRQVVHGKEGSIFLVTGQQVDYSIIASQAGATLNNLMSHAQELVAKLRSLQFDQREFVCLKFLVLFSLDVKNLENFQLVEGVQEQVNAALLDYTMCNYPQQTEKFGQLLLRLPEIRAISMQAEEYLYYKHLNGDVPYNNLLIEMLHAKRA

SEQ ID NO: 225 - protein, 164 aa, Artificial Sequence
  Protein domain fragment
KMADQTLFSIVEWARSSIFFRELKVDDQMKLLQNCWSELLILDHIYRQVVHGKEGSIFLVTGQQVDYSIIASQAGATLNNLMSHAQELVAKLRSLQFDQREFVCLKFLVLFSLDVKNLENFQLVEGVQEQVNAALLDYTMCNYPQQTEKFGQLLLRLPEIRAIS

SEQ ID NO: 226 - protein, 263 aa, Artificial Sequence
  Protein domain fragment
EGVRLDRVRGGRQKYKRRIDAENSPYLNPQLVQPAKKPYNKIVSHLLVAEPEKIYAMPDPTVPDSDIKALTTLCDLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTSTKAVQHFYNIKLEGKVPMHKLFLEMLEAKV

SEQ ID NO: 227 - protein, 159 aa, Artificial Sequence
  Protein domain fragment
DLADRELVVIIGWAKHIPGFSTLSLADQMSLLQSAWMEILILGVVYRSLSFEDELVYADDYIMDEDQSKLAGLLDLNNAILQLVKKYKSMKLEKEEFVTLKAIALANSDSMHIEDVEAVQKLQDVLHEALQDYEAGQHMEDPRRAGKMLMTLPLLRQTS

SEQ ID NO: 228 - protein, 313 aa, Artificial Sequence
  Protein domain fragment
CKEAGMREQCVLSEEQIRKKKIRKQQQQESQSQSQSPVGPQGSSSSASGPGASPGGSEAGSQGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE

SEQ ID NO: 229 - protein, 252 aa, Artificial Sequence
  Protein domain fragment
QGSGEGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE

SEQ ID NO: 230 - protein, 248 aa, Artificial Sequence
  Protein domain fragment
EGEGVQLTAAQELMIQQLVAAQLQCNKRSFSDQPKVTPWPLGADPQSRDARQQRFAHFTELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLSSVHSEQVFALRLQDKKLPPLLSEIWDVHE

SEQ ID NO: 231 - protein, 160 aa, Artificial Sequence
  Protein domain fragment
ELAIISVQEIVDFAKQVPGFLQLGREDQIALLKASTIEIMLLETARRYNHETECITFLKDFTYSKDDFHRAGLQVEFINPIFEFSRAMRRLGLDDAEYALLIAINIFSADRPNVQEPGRVEALQQPYVEALLSYTRIKRPQDQLRFPRMLMKLVSLRTLS